(19) United States
(12) Patent Application Publication
(10) Pub. No.: US 2025/0025582 A1
(43) Pub. Date: Jan. 23, 2025

(54) LIGANDS AND THEIR USE

(71) Applicant: THE UNIVERSITY OF SYDNEY, Sydney, NSW (AU)

(72) Inventors: Rachel CODD, Sydney, NSW (AU); James Liam WOOD, Sydney, NSW (AU); Christopher John McCormack BROWN, Sydney, NSW (AU)

(21) Appl. No.: 18/258,394

(22) PCT Filed: Dec. 22, 2021
(86) PCT No.: PCT/AU2021/051542

§ 371 Date: Jun. 20, 2023

(30) Foreign Application Priority Data
Dec. 22, 2020 (AU) 2020904791

(51) Int. Cl. A61K 51/04

(52) U.S. Cl. CPC A61K 51/0482; A61K 2121/00; A61K 2123/00

(57) ABSTRACT

This disclosure relates to compounds that include a first chelating ligand selective for ⁸⁹Zr linked by a linker group to a second chelating ligand selective for a radionuclide other than ⁸⁹Zr. Also disclosed are complexes, pharmaceutical agents and compositions comprising the compounds. The disclosure also provides methods for the use and production of the compounds, complexes, pharmaceutical agents and compositions.

### FIELD OF THE INVENTION

The present disclosure relates to a chelating ligand for a nuclide or nuclides of pharmaceutical potential, and complexes of the ligand with the nuclide or nuclides. The disclosure also relates to pharmaceutical agents, compositions and kits comprising the ligands and complexes, and to methods of use and production.

### BACKGROUND OF THE INVENTION

In therapy and imaging, certain metals are increasingly important, particularly with respect to neoplastic disorders. The metals of interest typically are able to emit radiation through radioactive decay from within the body to either serve as contrast agents improving the sensitivity of an imaging technique or for therapeutic purposes.

For some imaging techniques particular non-radioactive nuclides may also be useful. Radionuclides used in imaging or therapy are selected based upon a variety of properties that include the type of radiation emitted by the isotope and the half-life of the isotope.

For pharmaceutical use, radionuclides are typically formulated as a complex with an organic ligand that coordinates or binds the radionuclide with high affinity, typically by chelating through four or more binding sites. This high affinity binding assists increase the overall stability of the complex between the radionuclide and the multidentate ligand with the radionuclide, and may reduce leaching (metal loss from the disassociation of the complex) or trans-chelation (transfer of the metal to a different ligand or molecule) of the radionuclide from the complex upon administration.

Despite the ability to form multiple binding interactions, not all ligands capable of chelation are suitable to bind a given radionuclide for pharmaceutical use as a complex. Different ligands have different affinities and selectivities for different radionuclides. Accordingly, an appropriate ligand must be utilised in combination with the desired radionuclide.

For example, interest in the pharmaceutical applications of the radionuclide zirconium-89 (⁸⁹Zr) is growing. ⁸⁹Zr (beta-positive emitter (av), 0.396 MeV) has potential applications in positron emission tomography (PET) imaging. ⁸⁹Zr is of particular interest in immunological PET (immuno-PET) imaging due to its extended 3.3 d half-life which matches the circulation half-life of an antibody. In immuno-PET imaging, tumours are imaged based upon expression of tumour-associated antigens on tumour cells through the use of a radionuclide complex conjugated to an appropriate antibody. However, immuno-PET imaging often suffers from slow accumulation of the radionuclide-antibody conjugate in the target tissue. This means that radionuclides other than ⁸⁹Zr that can be used in PET imaging often do not have an appropriate half-life for immuno-PET imaging.

One potential issue with the presence of different chelating ligands in a compound or composition for complexation to a given radionuclide is the possibility for competing ligand-radionuclide interactions. Competing interactions between the radionuclide and the different chelating ligands mean that the formation of multiple complexes (eg one complex between the radionuclide and one chelating ligand as well as a further complex between the radionuclide and a different chelating ligand) is a potential concern, particularly if a further complex is of intermediate affinity. Such complexes of intermediate affinity are vulnerable to leaching of the radionuclide from the complex upon administration. In many cases, the concentration of such complexes of intermediate affinity can be reduced to appropriate levels through careful control of the stoichiometry and equilibration time prior to administration. However, the half-lives of radionuclides suitable for pharmaceutical use mean that equilibration times prior to administration should ideally be minimised.

There is a continuing need to develop ligands for nuclides of pharmaceutical, diagnostic and/or prognostic potential. There is also a need to develop pharmaceutically acceptable compounds able to chelate ⁸⁹Zr, preferably selectively chelate ⁸⁹Zr or one or more further nuclides of therapeutic potential.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect of the invention, there is provided a compound comprising:
a first chelating ligand (chelating ligand 1) selective for ⁸⁹Zr, and
a second chelating ligand (chelating ligand 2) selective for a radionuclide of pharmaceutical potential other than ⁸⁹Zr,
wherein the first and second chelating ligands are covalently linked by a linker group.

In some embodiments, the compound of the invention is a compound of Formula (I)

A-L-B (I)
wherein
A is a chelating ligand selective for ⁸⁹Zr,
B is a chelating ligand selective for a radionuclide of pharmaceutical potential other than ⁸⁹Zr, and
L is a linker group.

In another aspect, there is provided a compound of Formula (II)

Ch¹-L-Ch² (II)
wherein
Ch¹ comprises a radical of desferrioxamine B (DFOB);
Ch² comprises a radical of 1,4,7,10-tetraazacyclododecane-1,4,7,10-tetraacetic acid (DOTA); and
L is a linker group.

In a further aspect, there is provided a complex comprising a compound of the present invention and a metal. In some embodiments, the complex comprises two different metals.

In a further aspect, there is provided a pharmaceutical agent comprising a compound of the invention and a radionuclide of pharmaceutical potential. In some embodiments, the pharmaceutical agent comprises a compound and two different radionuclides of pharmaceutical potential.

In a further aspect, there is provided a composition comprising a compound, complex or pharmaceutical agent of the present invention and a pharmaceutically acceptable excipient.

In a further aspect there provided is a kit of parts, comprising in separate parts:
a compound, complex, pharmaceutical agent or composition of the present invention; and
instructions for its use in any of the methods of the invention.

The invention further relates to the use of such complexes, agents, compositions and kits in therapy, diagnosis and/or prognosis of disease. Thus accordingly, the compound, complex, therapeutic agent or composition of the invention may be used variously as a therapeutic agent, a diagnostic agent or a prognostic agent.

Also provided are methods of producing a compound, complex, pharmaceutical agent or composition of the present invention.

Any embodiment herein shall be taken to apply mutatis mutandis to any other embodiment unless specifically stated otherwise.

The present disclosure is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### Definitions

Unless otherwise herein defined, the following terms will be understood to have the general meanings which follow.

The term “C₁₋₆alkyl” refers to optionally substituted straight chain or branched chain hydrocarbon groups having from 1 to 6 carbon atoms. Examples include methyl (Me), ethyl (Et), propyl (Pr), isopropyl (i-Pr), butyl (Bu), isobutyl (i-Bu), sec-butyl (s-Bu), tert-butyl (t-Bu), pentyl, neopentyl, hexyl and the like. Unless the context requires otherwise, the term “C₁₋₆alkyl” also encompasses alkyl groups containing one less hydrogen atom such that the group is attached via two positions i.e. divalent. “C₁₋₄alkyl” and “C₁₋₃alkyl” including methyl, ethyl, propyl, isopropyl, n-butyl, iso-butyl, sec-butyl and tert-butyl are preferred with methyl being particularly preferred.

The term “C₂₋₆alkenyl” refers to optionally substituted straight chain or branched chain hydrocarbon groups having at least one double bond of either E or Z stereochemistry where applicable and 2 to 6 carbon atoms. Examples include vinyl, 1-propenyl, 1- and 2-butenyl and 2-methyl-2-propenyl. Unless the context requires otherwise, the term “C₂₋₆alkenyl” also encompasses alkenyl groups containing one less hydrogen atom such that the group is attached via two positions i.e. divalent. “C₂₋₄alkenyl” and “C₂₋₃alkenyl” including ethenyl, propenyl and butenyl are preferred with ethenyl being particularly preferred.

The term “C₂₋₆alkynyl” refers to optionally substituted straight chain or branched chain hydrocarbon groups having at least one triple bond and 2 to 6 carbon atoms. Examples include ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl and the like. Unless the context indicates otherwise, the term “C₂₋₆alkynyl” also encompasses alkynyl groups containing one less hydrogen atom such that the group is attached via two positions i.e. divalent. C₂₋₃alkynyl is preferred.

The term “C₃₋₁₀cycloalkyl” refers to non-aromatic cyclic groups having from 3 to 10 carbon atoms, including cyclopropyl, cyclobutyl, cyclopentyl, cyclohexyl, cycloheptyl, cyclooctyl, cyclononyl and cyclodecyl. It will be understood that cycloalkyl groups may be saturated such as cyclohexyl or unsaturated such as cyclohexenyl. C₃₋₆cycloalkyl such as cyclopropyl, cyclobutyl, cyclopentyl and cyclohexyl are preferred. Cycloalkyl groups also include polycyclic carbocycles and include fused, bridged and spirocyclic systems.

The terms “hydroxy” and “hydroxyl” refer to the group —OH.

The term “oxo” refers to the group ═O.

The term “C₁₋₆alkoxy” refers to an alkyl group as defined above covalently bound via an O linkage containing 1 to 6 carbon atoms, such as methoxy, ethoxy, propoxy, isoproxy, butoxy, tert-butoxy and pentoxy. “C₁₋₄alkoxy” and “C₁₋₃alkoxy” including methoxy, ethoxy, propoxy and butoxy are preferred with methoxy being particularly preferred.

The terms “haloC₁₋₆alkyl” and “C₁₋₆alkylhalo” refer to a C₁₋₆alkyl which is substituted with one or more halogens. HaloC₁₋₃alkyl groups are preferred, such as for example, —CH₂CF₃, and —CF₃.

The terms “haloC₁₋₆alkoxy” and “C₁₋₃alkoxyhalo” refer to a C₁₋₆alkoxy which is substituted with one or more halogens. C₁₋₃alkoxyhalo groups are preferred, such as for example, —OCF₃.

The term “carboxylate” or “carboxy” refers to the group —COO— or —COOH.

The term “ester” refers to a carboxyl group having the hydrogen replaced with, for example a C₁₋₆alkyl group (“carboxylC₁₋₆alkyl” or “alkylester”), an aryl or aralkyl group (“arylester” or “aralkylester”) and so on. CO₂C₁₋₃alkyl groups are preferred, such as for example, methylester (CO₂Me), ethylester (CO₂Et) and propylester (CO₂Pr) and includes reverse esters thereof (e.g. —OC(O)Me, —OC(O)Et and —OC(O)Pr).

The terms “cyano” and “nitrile” refer to the group —CN.

The term “nitro” refers to the group —NO₂.

The term “amino” refers to the group —NH₂.

The term “substituted amino” refers to an amino group having at least one hydrogen replaced with, for example a C₁₋₆alkyl group (“C₁₋₆alkylamino”), an aryl or aralkyl group (“arylamino”, “aralkylamino”) and so on. Substituted amino groups include “monosubstituted amino” (or “secondary amino”) groups, which refer to an amino group having a single hydrogen replaced with, for example a C₁₋₆alkyl group, an aryl or aralkyl group and so on. Preferred secondary amino groups include C₁₋₃alkylamino groups, such as for example, methylamino (NHMe), ethylamino (NHEt) and propylamino (NHPr). Substituted amino groups also include “disubstituted amino” (or “tertiary amino”) groups, which refer to amino groups having both hydrogens replaced with, for example C₁₋₆alkyl groups, which may be the same or different (“dialkylamino”), aryl and alkyl groups (“aryl(alkyl)amino”) and so on. Preferred tertiary amino groups include di(C₁₋₃alkyl)amino groups, such as for example, dimethylamino (NMe₂), diethylamino (NEt₂), dipropylamino (NPr₂) and variations thereof (e.g. N(Me)(Et) and so on).

The term “aldehyde” refers to the group —C(═O)H.

The terms “acyl” and “acetyl” refers to the group —C(O)CH₃.

The term “ketone” refers to a carbonyl group which may be represented by —C(O)—.

The term “substituted ketone” refers to a ketone group covalently linked to at least one further group, for example, a C₁₋₆alkyl group (“C₁₋₆alkylacyl” or “alkylketone” or “ketoalkyl”), an aryl group (“arylketone”), an aralkyl group (“aralkylketone”) and so on. C₁₋₃alkylacyl groups are preferred.

The term “amido” or “amide” refers to the group —C(O)NH₂.

The term “substituted amido” or “substituted amide” refers to an amido group having a hydrogen replaced with, for example a C₁₋₆alkyl group (“C₁₋₆alkylamido” or “C₁₋₆alkylamide”), an aryl (“arylamido”), aralkyl group (“aralkylamido”) and so on. C₁₋₃alkylamide groups are preferred, such as for example, methylamide (—C(O)NHMe), ethylamide (—C(O)NHEt) and propylamide (—C(O)NHPr) and includes reverse amides thereof (e.g. —NHMeC(O)—, —NHEtC(O)— and —NHPrC(O)—).

The term “disubstituted amido” or “disubstituted amide” refers to an amido group having the two hydrogens replaced with, for example a C₁₋₆alkyl group (“di(C₁₋₆alkyl)amido” or “di(C₁₋₆alkyl)amide”), an aralkyl and alkyl group (“alkyl(aralkyl)amido”) and so on. Di(C₁₋₃alkyl)amide groups are preferred, such as for example, dimethylamide (—C(O)NMe₂), diethylamide (—C(O)NEt₂) and dipropylamide ((—C(O)NPr₂) and variations thereof (e.g. —C(O)N(Me)Et and so on) and includes reverse amides thereof.

The term “thiol” refers to the group —SH.

The term “C₁₋₆alkylthio” refers to a thiol group having the hydrogen replaced with a C₁₋₆alkyl group. C₁₋₃alkylthio groups are preferred, such as for example, thiolmethyl, thiolethyl and thiolpropyl.

The terms “thioxo” refer to the group ═S.

The term “sulfinyl” refers to the group —S(═O)H.

The term “substituted sulfinyl” or “sulfoxide” refers to a sulfinyl group having the hydrogen replaced with, for example a C₁₋₆alkyl group (“C₁₋₆alkylsulfinyl” or “C₁₋₆alkylsulfoxide”), an aryl (“arylsulfinyl”), an aralkyl (“aralkyl sulfinyl”) and so on. C1-3alkylsulfinyl groups are preferred, such as for example, —SOmethyl, —SOethyl and —SOpropyl.

The term “sulfonyl” refers to the group —SO₂H.

The term “substituted sulfonyl” refers to a sulfonyl group having the hydrogen replaced with, for example a C₁₋₆alkyl group (“sulfonylC₁₋₆alkyl”), an aryl (“arylsulfonyl”), an aralkyl (“aralkylsulfonyl”) and so on. SulfonylC₁₋₃alkyl groups are preferred, such as for example, —SO₂Me, —SO₂Et and —SO₂Pr.

The term “sulfonylamido” or “sulfonamide” refers to the group —SO₂NH₂.

The term “substituted sulfonamido” or “substituted sulphonamide” refers to an sulfonylamido group having a hydrogen replaced with, for example a C₁₋₆alkyl group (“sulfonylamidoC₁₋₆alkyl”), an aryl (“arylsulfonamide”), aralkyl (“aralkylsulfonamide”) and so on. SulfonylamidoC₁₋₃alkyl groups are preferred, such as for example, —SO₂NHMe, —SO₂NHEt and —SO₂NHPr and includes reverse sulfonamides thereof (e.g. —NHSO₂Me, —NHSO₂Et and —NHSO₂Pr).

The term “disubstituted sufonamido” or “disubstituted sulphonamide” refers to an sulfonylamido group having the two hydrogens replaced with, for example a C₁₋₆alkyl group, which may be the same or different (“sulfonylamidodi(C₁₋₆alkyl)”), an aralkyl and alkyl group (“sulfonamido(aralkyl)alkyl”) and so on. Sulfonylamidodi(C₁₋₃alkyl) groups are preferred, such as for example, —SO₂NMe₂, —SO₂NEt₂ and —SO₂NPr₂ and variations thereof (e.g. —SO₂N(Me)Et and so on) and includes reserve sulfonamides thereof (e.g. —N(Me)SO₂Me and so on).

The term “sulfate” refers to the group OS(O)₂OH and includes groups having the hydrogen replaced with, for example a C₁₋₆alkyl group (“alkylsufates”), an aryl (“arylsulfate”), an aralkyl (“aralkylsulfate”) and so on. C₁₋₃sulfates are preferred, such as for example, OS(O)₂OMe, OS(O)₂OEt and OS(O)₂OPr.

The term “sulfonate” refers to the group SO₃H and includes groups having the hydrogen replaced with, for example a C₁₋₆alkyl group (“alkylsulfonate”), an aryl (“arylsulfonate”), an aralkyl (“aralkylsulfonate”) and so on. C₁₋₃sulfonates are preferred, such as for example, SO₃Me, SO₃Et and SO₃Pr.

The term “aryl” refers to a carbocyclic (non-heterocyclic) aromatic ring or mono-, bi- or tri-cyclic ring system. The aromatic ring or ring system is generally composed of 6 to 10 carbon atoms. Examples of aryl groups include but are not limited to phenyl, biphenyl, naphthyl and tetrahydronaphthyl. 6-membered aryls such as phenyl are preferred. The term “alkylaryl” refers to C₁₋₄alkylaryl such as benzyl.

The term “alkoxyaryl” refers to C₁₋₆alkyloxyaryl such as benzyloxy.

The term “heterocyclyl” refers to a moiety obtained by removing a hydrogen atom from a ring atom of a heterocyclic compound which moiety has from 3 to 10 ring atoms (unless otherwise specified), of which 1, 2, 3 or 4 are ring heteroatoms each heteroatom being independently selected from O, S and N. Heterocyclyl groups include monocyclic and polycyclic (such as bicyclic) ring systems, such as fused, bridged and spirocyclic systems, provided at least one of the rings of the ring system contains at least one heteroatom.

In this context, the prefixes 3-, 4-, 5-, 6-, 7-, 8-, 9- and 10-membered denote the number of ring atoms, or range of ring atoms, whether carbon atoms or heteroatoms. For example, the term “3-10 membered heterocyclyl”, as used herein, pertains to a heterocyclyl group having 3, 4, 5, 6, 7, 8, 9 or 10 ring atoms. Examples of heterocyclyl groups include 5-6-membered monocyclic heterocyclyls and 9-10 membered fused bicyclic heterocyclyls.

Examples of monocyclic heterocyclyl groups include, but are not limited to, those containing one nitrogen atom such as aziridine (3-membered ring), azetidine (4-membered ring), pyrrolidine (tetrahydropyrrole), pyrroline (e.g., 3-pyrroline, 2,5-dihydropyrrole), 2H-pyrrole or 3H-pyrrole (isopyrrole, isoazole) or pyrrolidinone (5-membered rings), piperidine, dihydropyridine, tetrahydropyridine (6-membered rings), and azepine (7-membered ring); those containing two nitrogen atoms such as imidazoline, pyrazolidine (diazolidine), imidazoline, pyrazoline (dihydropyrazole) (5-membered rings), piperazine (6-membered ring); those containing one oxygen atom such as oxirane (3-membered ring), oxetane (4-membered ring), oxolane (tetrahydrofuran), oxole (dihydrofuran) (5-membered rings), oxane (tetrahydropyran), dihydropyran, pyran (6-membered rings), oxepin (7-membered ring); those containing two oxygen atoms such as dioxolane (5-membered ring), dioxane (6-membered ring), and dioxepane (7-membered ring); those containing three oxygen atoms such as trioxane (6-membered ring); those containing one sulfur atom such as thiirane (3-membered ring), thietane (4-membered ring), thiolane (tetrahydrothiophene) (5-membered ring), thiane (tetrahydrothiopyran) (6-membered ring), thiepane (7-membered ring); those containing one nitrogen and one oxygen atom such as tetrahydrooxazole, dihydrooxazole, tetrahydroisoxazole, dihydroisoxazole (5-membered rings), morpholine, tetrahydrooxazine, dihydrooxazine, oxazine (6-membered rings); those containing one nitrogen and one sulfur atom such as thiazoline, thiazolidine (5-membered rings), thiomorpholine (6-membered ring); those containing two nitrogen and one oxygen atom such as oxadiazine (6-membered ring); those containing one oxygen and one sulfur atom such as: oxathiole (5-membered ring) and oxathiane (thioxane) (6-membered ring); and those containing one nitrogen, one oxygen and one sulfur atom such as oxathiazine (6-membered ring).

Heterocyclyls encompass aromatic heterocyclyls and non-aromatic heterocyclyls. Such groups may be substituted or unsubstituted.

The term “aromatic heterocyclyl” may be used interchangeably with the term “heteroaromatic” or the term “heteroaryl” or “hetaryl”. The heteroatoms in the aromatic heterocyclyl group may be independently selected from N, S and O. The aromatic heterocyclyl groups may comprise 1, 2, 3, 4 or more ring heteroatoms. In the case of fused aromatic heterocyclyl groups, only one of the rings may contain a heteroatom and not all rings must be aromatic.

“Heteroaryl” is used herein to denote a heterocyclic group having aromatic character and embraces aromatic monocyclic ring systems and polycyclic (e.g. bicyclic) ring systems containing one or more aromatic rings. The term aromatic heterocyclyl also encompasses pseudoaromatic heterocyclyls. The term “pseudoaromatic” refers to a ring system which is not strictly aromatic, but which is stabilized by means of delocalization of electrons and behaves in a similar manner to aromatic rings. The term aromatic heterocyclyl therefore covers polycyclic ring systems in which all of the fused rings are aromatic as well as ring systems where one or more rings are non-aromatic, provided that at least one ring is aromatic. In polycyclic systems containing both aromatic and non-aromatic rings fused together, the group may be attached to another moiety by the aromatic ring or by a non-aromatic ring.

Examples of heteroaryl groups are monocyclic and bicyclic groups containing from five to ten ring members. The heteroaryl group can be, for example, a five membered or six membered monocyclic ring or a bicyclic structure formed from fused five and six membered rings or two fused six membered rings or two fused five membered rings. Each ring may contain up to about four heteroatoms typically selected from nitrogen, sulphur and oxygen. The heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Aromatic heterocyclyl groups may be 5-membered or 6-membered mono-cyclic aromatic ring systems.

Examples of 5-membered monocyclic heteroaryl groups include but are not limited to furanyl, thienyl, pyrrolyl, oxazolyl, oxadiazolyl (including 1,2,3 and 1,2,4 oxadiazolyls and furazanyl i.e. 1,2,5-oxadiazolyl), thiazolyl, isoxazolyl, isothiazolyl, pyrazolyl, imidazolyl, triazolyl (including 1,2,3, 1,2,4 and 1,3,4 triazolyls), oxatriazolyl, tetrazolyl, thiadiazolyl (including 1,2,3 and 1,3,4 thiadiazolyls) and the like.

Examples of 6-membered monocyclic heteroaryl groups include but are not limited to pyridinyl, pyrimidinyl, pyridazinyl, pyrazinyl, triazinyl, pyranyl, oxazinyl, dioxinyl, thiazinyl, thiadiazinyl and the like. Examples of 6-membered aromatic heterocyclyls containing nitrogen include pyridyl (1 nitrogen), pyrazinyl, pyrimidinyl and pyridazinyl (2 nitrogens).

Aromatic heterocyclyl groups may also be bicyclic or polycyclic heteroaromatic ring systems such as fused ring systems (including purine, pteridinyl, naphthyridinyl, 1H thieno[2,3-c]pyrazolyl, thieno[2,3-b]furyl and the like) or linked ring systems (such as oligothiophene, polypyrrole and the like). Fused ring systems may also include aromatic 5-membered or 6-membered heterocyclyls fused to carbocyclic aromatic rings such as phenyl, naphthyl, indenyl, azulenyl, fluorenyl, anthracenyl and the like, such as 5-membered aromatic heterocyclyls containing nitrogen fused to phenyl rings, 5-membered aromatic heterocyclyls containing 1 or 2 nitrogens fused to phenyl ring.

A bicyclic heteroaryl group may be, for example, a group selected from: a) a benzene ring fused to a 5- or 6-membered ring containing 1, 2 or 3 ring heteroatoms; b) a pyridine ring fused to a 5- or 6-membered ring containing 1, 2 or 3 ring heteroatoms; c) a pyrimidine ring fused to a 5- or 6-membered ring containing 1 or 2 ring heteroatoms; d) a pyrrole ring fused to a 5- or 6-membered ring containing 1, 2 or 3 ring heteroatoms; e) a pyrazole ring fused to a 5- or 6-membered ring containing 1 or 2 ring heteroatoms; f) an imidazole ring fused to a 5- or 6-membered ring containing 1 or 2 ring heteroatoms; g) an oxazole ring fused to a 5- or 6-membered ring containing 1 or 2 ring heteroatoms; h) an isoxazole ring fused to a 5- or 6-membered ring containing 1 or 2 ring heteroatoms; i) a thiazole ring fused to a 5- or 6-membered ring containing 1 or 2 ring heteroatoms; j) an isothiazole ring fused to a 5- or 6-membered ring containing 1 or 2 ring heteroatoms; k) a thiophene ring fused to a 5- or 6-membered ring containing 1, 2 or 3 ring heteroatoms; l) a furan ring fused to a 5- or 6-membered ring containing 1, 2 or 3 ring heteroatoms; m) a cyclohexyl ring fused to a 5- or 6-membered ring containing 1, 2 or 3 ring heteroatoms; and n) a cyclopentyl ring fused to a 5- or 6-membered ring containing 1, 2 or 3 ring heteroatoms.

Particular examples of bicyclic heteroaryl groups containing a five membered ring fused to another five membered ring include but are not limited to imidazothiazole (e.g. imidazo[2,1-b]thiazole) and imidazoimidazole (e.g. imidazo[1,2-a]imidazole).

Particular examples of bicyclic heteroaryl groups containing a six membered ring fused to a five membered ring include but are not limited to benzofuran, benzothiophene, benzimidazole, benzoxazole, isobenzoxazole, benzisoxazole, benzothiazole, benzisothiazole, isobenzofuran, indole, isoindole, indolizine, indoline, isoindoline, purine (e.g., adenine, guanine), indazole, pyrazolopyrimidine (e.g. pyrazolo[1,5-a]pyrimidine), benzodioxole and pyrazolopyridine (e.g. pyrazolo[1,5-a]pyridine) groups. A further example of a six membered ring fused to a five membered ring is a pyrrolopyridine group such as a pyrrolo[2,3-b]pyridine group.

Particular examples of bicyclic heteroaryl groups containing two fused six membered rings include but are not limited to quinoline, isoquinoline, chroman, thiochroman, chromene, isochromene, isochroman, benzodioxan, quinolizine, benzoxazine, benzodiazine, pyridopyridine, quinoxaline, quinazoline, cinnoline, phthalazine, naphthyridine and pteridine groups.

Examples of heteroaryl groups containing an aromatic ring and a non-aromatic ring include tetrahydronaphthalene, tetrahydroisoquinoline, tetrahydroquinoline, dihydrobenzothiophene, dihydrobenzofuran, 2,3-dihydro-benzo[1,4]dioxine, benzo[1,3]dioxole, 4,5,6,7-tetrahydrobenzofuran, indoine, isoindoline and indane groups.

Examples of aromatic heterocyclyls fused to carbocyclic aromatic rings may therefore include but are not limited to benzothiophenyl, indolyl, isoindolyl, benzofuranyl, isobenzofuranyl, benzimidazolyl, indazolyl, benzoxazolyl, benzisoxazolyl, isobenzoxazoyl, benzothiazolyl, benzisothiazolyl, quinolinyl, isoquinolinyl, quinoxalinyl, quinazolinyl, cinnolinyl, benzotriazinyl, phthalazinyl, carbolinyl and the like.

The term “non-aromatic heterocyclyl” encompasses optionally substituted saturated and unsaturated rings which contain at least one heteroatom selected from the group consisting of N, S and O. The ring may contain 1, 2 or 3 heteroatoms. The ring may be a monocyclic ring or part of a polycyclic ring system. Polycyclic ring systems include fused rings and spirocycles. Not every ring in a non-aromatic heterocyclic polycyclic ring system must contain a heteroatom, provided at least one ring contains one or more heteroatoms.

Non-aromatic heterocyclyls may be 3-7 membered mono-cyclic rings.

Examples of 5-membered non-aromatic heterocyclyl rings include 2H-pyrrolyl, 1-pyrrolinyl, 2-pyrrolinyl, 3-pyrrolinyl, pyrrolidinyl, 1-pyrrolidinyl, 2-pyrrolidinyl, 3-pyrrolidinyl, tetrahydrofuranyl, tetrahydrothiophenyl, pyrazolinyl, 2-pyrazolinyl, 3-pyrazolinyl, pyrazolidinyl, 2-pyrazolidinyl, 3-pyrazolidinyl, imidazolidinyl, 3-dioxalanyl, thiazolidinyl, isoxazolidinyl, 2-imidazolinyl and the like.

Examples of 6-membered non-aromatic heterocyclyls include piperidinyl, piperidinonyl, pyranyl, dihyrdopyranyl, tetrahydropyranyl, 2H pyranyl, 4H pyranyl, thianyl, thianyl oxide, thianyl dioxide, piperazinyl, diozanyl, 1,4-dioxinyl, 1,4-dithianyl, 1,3,5-triozalanyl, 1,3,5-trithianyl, 1,4-morpholinyl, thiomorpholinyl, 1,4-oxathianyl, triazinyl, 1,4-thiazinyl and the like.

Examples of 7-membered non-aromatic heterocyclyis include azepanyl, oxepanyl, thiepanyl and the like.

Non-aromatic heterocyclyl rings may also be bicyclic heterocyclyl rings such as linked ring systems (for example uridinyl and the like) or fused ring systems.

Fused ring systems include non-aromatic 5-membered, 6-membered or 7-membered heterocyclyls fused to carbocyclic aromatic rings such as phenyl, naphthyl, indenyl, azulenyl, fluorenyl, anthracenyl and the like. Examples of non-aromatic 5-membered, 6-membered or 7-membered heterocyclyis fused to carbocyclic aromatic rings include indolinyl, benzodiazepinyl, benzazepinyl, dihydrobenzofuranyl and the like.

The term “halo” refers to fluoro, chloro, bromo or iodo.

Unless otherwise defined, the term “optionally substituted” or “optional substituent” as used herein refers to a group which may or may not be further substituted with 1, 2, 3, 4 or more groups, preferably 1, 2 or 3, more preferably 1 or 2 groups selected from the group consisting of C₁₋₆alkyl, C₂₋₆alkenyl, C₂₋₆alkynyl, C₃₋₈cycloalkyl, hydroxyl, oxo, C₁₋₆alkoxy, aryloxy, C₁₋₆alkoxyaryl, halo, C₁₋₆alkylhalo (such as CF₃), C₁₋₆alkoxyhalo (such as OCF₃), carboxyl, esters, cyano, nitro, amino, substituted amino, disubstituted amino, acyl, ketones, substituted ketones, amides, aminoacyl, substituted amides, disubstituted amides, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arylC₁₋₆alkyl, heterocyclyl and heteroaryl wherein each alkyl, alkenyl, alkynyl, cycloalkyl, aryl and heterocyclyl and groups containing them may be further optionally substituted. Optional substituents in the case of heterocycles containing N may also include but are not limited to C₁₋₆alkyl i.e. N—C₁₋₃alkyl, more preferably methyl particularly N-methyl.

For optionally substituted “C₁₋₆alkyl”, “C₂₋₆alkenyl” and “C₂₋₆alkynyl”, the optional substituent or substituents are preferably selected from halo, aryl, heterocyclyl, C₃₋₈cycloalkyl, C₁₋₆alkoxy, hydroxyl, oxo, aryloxy, haloC₁₋₆alkyl, haloC₁₋₆alkoxyl and carboxyl. Each of these optional substituents may also be optionally substituted with any of the optional substituents referred to above, where nitro, amino, substituted amino, cyano, heterocyclyl (including non-aromatic heterocyclyl and heteroaryl), C₁₋₆alkyl, C₂₋₆alkenyl, C₂₋₆alkynyl, C₁₋₆alkoxyl, haloC₁₋₆alkyl, haloC₁₋₆alkoxy, halo, hydroxyl and carboxyl are preferred.

In the case of hybrid naming of substituent radicals, such as haloalkyl and alkylaryl, it is to be understood that no direction in the order of groups is intended so the point of attachment may be to any of the moieties included in the hybrid radical. For example, the terms “alkylaryl” and “arylalkyl” are intended to refer to the same group and the point of attachment may be via the alkyl or the aryl moiety (or both in the case of diradical species).

By ‘chelating ligand’ it is meant a functional group or collection of functional groups suitable for binding a metal atom to form a multidentate coordination complex. The chelating ligand may form 2, 3, 4 or more coordination bonds with the metal atom, and may therefore comprise 2, 3, 4 or more ligand moieties. The collection of functional groups in a chelating ligand may comprise differing functional groups within the one collection (for instance, both carboxylate and hydroxyl functional groups may be present within the one chelating ligand).

The depiction of the hydroxamic acid moiety as

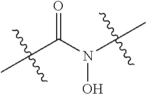 [SMILES: *C(=O)N(*)O]

will be understood to include both the forward and reverse (or retro) hydroxamic acid. Thus, the structure R^{a}—R¹—R^{b}, where R¹ is

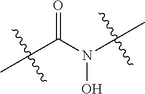 [SMILES: *C(=O)N(*)O]

will be understood to include both

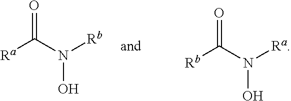

By ‘nuclide’ it is meant an isotope of a metal and may undergo radioactive decay or otherwise.

By ‘radionuclide’ it is meant an isotope of a metal that undergoes radioactive decay.

As used herein, ‘pharmaceutical potential’ includes therapeutic, diagnostic and/or prognostic potential. The nuclides of pharmaceutical potential referred to herein may be in any suitable oxidation state for the pharmaceutical use and to form a stable complex with the compound.

As used herein, except where the context requires otherwise, the term “comprise” and variations of the term, such as “comprising”, “comprises” and “comprised”, are not intended to exclude further additives, components, integers or steps.

It must be noted that as used herein and in the appended claims, the singular forms “a”, “an” and “the” include plural reference unless the context clearly dictates otherwise.

Thus, for example, a reference to “a salt” may include a plurality of salts and a reference to “at least one heteroatom” may include one or more heteroatoms, and so forth.

The term “and/or” can mean “and” or “or”.

The term “(s)” following a noun contemplates the singular or plural form, or both.

Various features of the invention are described with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, may mean an amount within ±25%, ±10%, ±5%, ±1% or ±0.1% of that value.

By ‘metal’ it is meant a metal in any oxidation state. The person skilled in the art will appreciate that metal may refer to metal in a suitable oxidation state for use, for instance, such that the metal is suitable for complexation to a compound of the invention and/or soluble in a mixture.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

Embodiments of the invention will be further described with reference to the following non-limiting drawings, in which:

FIG. 1 shows generic features of different forms of the compounds of the invention and specific embodiments of those generic features. Specifically:

FIG. 1a shows a compound that is a two-component system form of the invention, where one of the components is a chelating ligand selective for ⁸⁹Zr and the other component is a chelating ligand selective for a nuclide of pharmaceutical potential other than ⁸⁹Zr. FIG. 1a also shows an embodiment of the two-component system where the chelating ligand selective for ⁸⁹Zr is based upon DFOB and the chelating ligand selective for a nuclide of pharmaceutical potential other than ⁸⁹Zr is based upon DOTA. This embodiment of the two-component system is further discussed in Example 1 and Example 2.

FIG. 1b shows a compound that is a three-component system form of the invention, where one of the components is a chelating ligand selective for ⁸⁹Zr and one of the components is a chelating ligand selective for a nuclide of pharmaceutical potential other than ⁸⁹Zr. The further component in the three-component system shown in FIG. 1b is a linker. The linker may optionally comprise a targeting moiety or a substituent capable of conjugation to a targeting moiety. If present, such a feature can result in biological targeting of the compound. FIG. 1b also shows an embodiment of the three-component system where a linker is present and the chelating ligand selective for ⁸⁹Zr is based upon DFOB, the chelating ligand selective for a nuclide of pharmaceutical potential other than ⁸⁹Zr is based upon DOTA and the linker is based upon lysine. The linker based upon lysine comprises a free amine group, which is a substituent capable of conjugation to a targeting moiety or being further modified to install a functional group more distant from the backbone of the compound to enable the conjugation of a targeting moiety. This embodiment of the three-component system is further discussed in Example 3 and Example 4.

FIG. 1c shows a compound that is a three-component system form of the invention, where one of the components is a chelating ligand selective for ⁸⁹Zr and one of the components is a chelating ligand selective for a nuclide of pharmaceutical potential other than ⁸⁹Zr. The further component in the three-component system shown in FIG. 1c is a moiety to enhance ⁸⁹Zr affinity of the chelating ligand selective for ⁸⁹Zr.

FIG. 1d shows a compound that is a four-component system form of the invention, where one of the components is a chelating ligand selective for ⁸⁹Zr, one of the components is a chelating ligand selective for a nuclide of pharmaceutical potential other than ⁸⁹Zr, one of the components is a linker and the other component is a moiety to enhance ⁸⁹Zr affinity of the chelating ligand selective for ⁸⁹Zr. The linker may optionally comprise a targeting moiety or a substituent capable of conjugation to a targeting moiety or being further modified to install a functional group more distant from the backbone of the compound to enable the conjugation of a targeting moiety. If present, such a feature can result in biological targeting of the compound. FIG. 1d also shows an embodiment of the four-component system where the chelating ligand selective for ⁸⁹Zr is based upon DFOB, the chelating ligand selective for a nuclide of pharmaceutical potential other than ⁸⁹Zr is based upon DOTA, the linker is based upon lysine and the moiety to enhance ⁸⁹Zr affinity of the chelating ligand selective for ⁸⁹Zr is based upon 5-((5-aminopentyl)(hydroxy)amino)-5-oxopentanoic acid (PPH). The linker based upon lysine comprises a free amine group, which is a substituent capable of conjugation to a targeting moiety or being further modified to install a functional group more distant from the backbone of the compound to enable the conjugation of a targeting moiety. This embodiment of the three-component system is further discussed in Example 5.

FIG. 2a shows liquid chromatography-mass spectrometry spectra from a solution of the semi-purified two-component system of Example 1 and Example 2: DFOB-DOTA (2) of Example 1 shown as total ion current (upper) or as EIC traces (lower) (black).

FIG. 2b shows liquid chromatography-mass spectrometry spectra from a solution of the semi-purified two-component system of Example 2: DFOB-DOTA (2) loaded with Zr(IV) to form Zr(IV)-2 of Example 2a shown as total ion current (upper) or as EIC traces (lower) (black). The EIC traces (black) correspond with Zr(IV)-DFOB-DOTA (Zr(IV)-2) as a complex with 1:1 metal:ligand stoichiometry. Signals with EIC values corresponding with Zr(IV)2-DFOB-DOTA ([M]2+) (grey) were at baseline, which supports the 1:1 stoichiometry.

FIG. 2c shows liquid chromatography-mass spectrometry spectra from a solution of the semi-purified two-component system of Example 2: DFOB-DOTA (2) loaded with Lu(III) to form Lu(III)-2 of Example 2b, shown as total ion current (upper) or as EIC traces (lower) (black). The EIC traces (black) correspond Lu(III)-DFOB-DOTA (Lu(III)-2) as a complex with 1:1 metal:ligand stoichiometry. Signals with EIC values corresponding with Lu(III)2-DFOB-DOTA ([M+2H]2+) (grey) were at baseline, which supports the 1:1 stoichiometry.

FIG. 3a shows mass spectrometry spectra from peak maxima of the two-component system of DFOB-DOTA (2) of Example 1 shown as experimental (upper) or calculated (theoretical) (lower) data.

FIG. 3b shows mass spectrometry spectra from peak maxima of the two-component system of Example 2: DFOB-DOTA (2) loaded with Zr(IV) to form Zr(IV)-2 of Example 2a shown as experimental (upper) or calculated (theoretical) (lower) data.

FIG. 3c shows mass spectrometry spectra from peak maxima of the two-component system of Example 2: DFOB-DOTA (2) loaded with Lu(III) to form Lu(III)-2 of Example 2b shown as experimental (upper) or calculated (theoretical) (lower) data.

FIG. 4a-c shows liquid chromatography-mass spectrometry from solutions of the HPLC-purified three-component system of Example 3 and Example 4: DFOB-L-LYS-DOTA (3) of Example 3 (FIG. 4a) or DFOB-L-LYS-DOTA (3) loaded with Zr(IV) to form Zr(IV)-3 of Example 4a (FIG. 4b) or Lu(III) to form Lu(III)-3 of Example 4b (FIG. 4c), shown as total ion current (upper) or as EIC traces (lower). The EIC traces correspond with Zr(IV)-DFOB-L-LYS-DOTA (Zr(IV)-3) or Lu(III)-DFOB-L-LYS-DOTA (Lu(III)-3) as complexes with 1:1 metal:ligand stoichiometry. Signals with EIC values corresponding with Zr(IV)2-DFOB-L-LYS-DOTA ([M]2+) or Lu(III)2-DFOB-L-LYS-DOTA ([M+2H]2+) (grey in respective panels) were at baseline, which supports the 1:1 stoichiometry.

FIG. 5a-c shows mass spectrometry from peak maxima of the three-component system of Example 3 and Example 4: DFOB-L-LYS-DOTA (3) of Example 3 (FIG. 5a) or DFOB-L-LYS-DOTA (3) loaded with Zr(IV) to form Zr(IV)-3 of Example 4a (FIG. 5b); or Lu(III) to form Lu(III)-3 of Example 4b (FIG. 5c), shown as experimental (upper) or calculated (theoretical) (lower) data.

FIG. 6 shows liquid chromatography-mass spectrometry from solutions of the semi-purified four-component system of Example 5: DFOB-PPH-L-LYS-DOTA (4), shown as total ion current (upper) or as EIC traces for the [M+3H]3+(black) or [M+4H]4+(grey) adducts (lower). This complex is predicted to label with metal ions in a fashion similar to the two-component system and the three-component system.

FIG. 7 shows the structure of the forward hydroxamic acid 5-((5-aminopentyl)(hydroxy)amino)-5-oxopentanoic acid (PPH) and the corresponding system DFOB-PPH-L-LYS-DOTA (4) is shown in FIG. 7, together with the equivalent reverse-hydroxamic acid 4-(6-amino-N-hydroxyhexanamido)butanoic acid (retro-PPH) and the cognate four-component system DFOB-retro-PPH-L-LYS-DOTA (retro-4).

FIG. 8 shows a liquid chromatography-mass spectrometry spectrum from the solution of the semi-purified product of Example 9 and mass spectrometry from the peak maximum of the semi-purified product of Example 9.

FIG. 9 shows a liquid chromatography-mass spectrometry (TIC) spectrum from a semi-pure reaction mixture containing DFOB-L-LYS-EPS-PEG4-DOTA of Example 10.

FIG. 10 shows the MS isotope pattern from the LC signal at 7.52 min (FIG. 9), corresponding with DFOB-L-LYS-EPS-PEG4-DOTA of Example 10.

FIG. 11 shows selected ion monitoring (EIC, m/z=661.885) corresponding with the [M+2H]²⁺ adduct of DFOB-L-LYS-EPS-PEG4-DOTA of Example 10.

FIG. 12 shows selected ion monitoring (EIC, m/z=441.59) corresponding with the [M+3H]³⁺ adduct of DFOB-L-LYS-EPS-PEG4-DOTA of Example 10.

FIG. 13 shows the liquid chromatography-mass spectrometry (TIC) spectrum from a reaction mixture containing NCS-Activated DFOB-L-LYS-EPS-PEG4-DOTA (Compound D2) of Example 11.

FIG. 14 shows the MS isotope pattern from the LC signal at 11.171-12.032 min (FIG. 13), corresponding with NCS-Activated DFOB-L-LYS-EPS-PEG4-DOTA (Compound D2) of Example 11.

FIG. 15 shows the selected ion monitoring (SIM, m/z=757.9) corresponding with the [M+2H]²⁺ adduct of NCS-Activated DFOB-L-LYS-EPS-PEG4-DOTA (Compound D2) of Example 11.

FIG. 16 shows the selected ion monitoring (SIM, m/z=505.6) corresponding with the [M+3H]³⁺ adduct of NCS-Activated DFOB-L-LYS-EPS-PEG4-DOTA (Compound D2) of Example 11.

FIG. 17 shows the relative cell-bound fraction for DOTA-mAb[inLu] at 30 min, 1 h and 2 h.

FIG. 18 shows the relative cell-bound fraction for Compound D2-mAb[¹Lu] at 30 min, 1 h and 2 h.

FIG. 19 shows the relative cell-bound fraction for DFOB-mAb[⁸⁹Zr] at 30 min, 1 h and 2 h.

FIG. 20 shows the relative cell-bound fraction for Compound D2-mAb[⁸⁹Zr] at 30 min, 1 h and 2 h.

FIG. 21 shows the coronal PET images of Compound D2-mAb[⁸⁹Zr](upper row) or DFOB-mAb[⁸⁹Zr] (lower row) at 4 h, 24 h or 48 h.

FIG. 22 shows the in vivo biodistribution of DFO-mAb[⁸⁹Zr] and Compound D2-mAb[⁸⁹Zr] 48 hours post-injection as determined by ROI analysis of PET images.

FIG. 23 shows the ex vivo biodistribution of DFO-mAb[⁸⁹Zr], DFO-mAb[¹Lu], Compound D2-mAb[⁸⁹Zr] and Compound D2-mAb[¹⁷⁷Lu] in the tumour 48 hours post-injection as determined by ex vivo gamma counting.

### DETAILED DESCRIPTION OF THE EMBODIMENTS

The present invention is directed towards a compound comprising two different chelating ligands, where each chelating ligand is selected to form a complex of sufficient affinity and/or selectivity for pharmaceutical use with different nuclides.

The inventors have developed a compound comprising two different chelating ligands, whereupon exposure to a suitable radionuclide of pharmaceutical potential leads to formation of a single stable complex. Surprisingly, the complex does not form as a mixture of coordination isomers with different metal binding modes, despite the potential binding sites on the compound. Furthermore, exposure to a different suitable radionuclide of pharmaceutical potential leads to formation of a different single stable complex bound through a different chelating ligand. Surprisingly, this different complex does not form as a mixture of binding modes, despite the potential binding sites on the compound. Furthermore, the compound is capable of forming a complex with two different metals, where each metal is bound through a different chelating ligand. Surprisingly, this complex also does not form as a mixture of binding modes, despite the mixture of potential binding sites on the compound.

Prior to the present invention it was thought that a potential issue with a compound or a composition that features different chelating ligands for complexation to a given nuclide was the possibility for deleterious performance as a result of the presence of different chelating ligands. It was thought that a given radionuclide could potentially bind to both chelating ligands to different extents, which could pose difficulties in ensuring robust radiolabelling procedures. For example, it was thought that such interactions could disrupt the typical affinity of the chelating ligand to a given nuclide, particularly if the different chelating ligands were included in the same compound, such that the local concentration of the other chelating ligand is higher. Advantageously, at least in preferred embodiments of the invention, each chelating ligand is able to bind its target nuclide with sufficient affinity and selectively despite the presence of another potential chelator in the same compound.

In some circumstances it would be advantageous to administer to a subject more than one metal of pharmaceutical potential at or near the same time, including more than one radionuclide of pharmaceutical potential. However, prior to the present invention it was also thought co-administration of more than one nuclide of pharmaceutical potential would present issues stemming from the different pharmacokinetics of suitable ligands, complicating administration. Advantageously, the compounds of the current invention are capable of complexation to more than one metal, including radionuclides, of pharmaceutical potential. This strategy simplifies the pharmacokinetics of the administration of more than one metal of pharmaceutical potential at or near the same time. However, ligands for nuclides, including radionuclides, suitable for pharmaceutical use need to be optimised for both pharmacokinetics and complexation to the nuclide(s) of pharmaceutical potential. Any structural changes to such an optimised ligand structure can disrupt the pharmacokinetics and/or the affinity of the ligand complex, potentially making the complex less suitable as a pharmaceutical. Surprisingly, despite changes to the structure of the ligand and the compound, a complex of the compound of the current invention with a metal of pharmaceutical potential is suitable for use as a pharmaceutical. This is despite the presence of different ligands on the compound, and includes when the compound is complexed with more than one metal of pharmaceutical potential (for instance a metal, including a radionuclide, of diagnostic potential and a metal, including a radionuclide, of therapeutic potential).

In one aspect of the invention, there is provided a compound comprising:
a first chelating ligand selective for ⁸⁹Zr, and
a second chelating ligand selective for a nuclide of pharmaceutical potential other than ⁸⁹Zr,
wherein the first and second chelating ligands are linked by a linker group.

The first and second chelating ligands may be linked by any suitable means. In some embodiments, the linker is a covalent linker.

In some embodiments, the compound of the invention is a compound of Formula (I)

A-L-B (I)
wherein
A is the first chelating ligand is selective for ⁸⁹Zr,
B is the second chelating ligand selective for a radionuclide of pharmaceutical potential other than ⁸⁹Zr, and
L is a linker group.

In some embodiments, the two different chelating ligands differ in their affinity and/or selectivity for a nuclide of pharmaceutical potential such that after an appropriate equilibration time the nuclide substantially only forms a stable complex with a single chelating ligand of the compound.

By ‘substantially’ in this context it is meant that at least 90% of the nuclide that is complexed to the compound is bound through the chelating ligand selective for ⁸⁹Zr or at least 90% of the nuclide that is complexed to the compound is bound through the chelating ligand selective for a nuclide of pharmaceutical potential other than ⁸⁹Zr.

By ‘equilibration time’ it is meant the time from which the nuclide is introduced to the compound. Typically, both the nuclide and compound will be dissolved in solution prior to introduction of the nuclide to the compound. The person skilled in the art will appreciate that equilibrium may be affected by factors such as concentration, temperature, pH, the presence of competing ions, the presence of additional solvents etc.

By ‘stable complex’ it is meant that a complex between the nuclide and chelating ligand does not suffer from disassociation that makes it unsuitable for pharmaceutical use. Complex stability may be quantified through disassociation constants such as K_{d}.

In some embodiments, after an appropriate equilibration time at least 95% of the nuclide that is complexed to the compound is bound through the chelating ligand selective for ⁸⁹Zr or at least 95% of the nuclide that is complexed to the compound is bound through the chelating ligand selective for a nuclide of pharmaceutical potential other than ⁸⁹Zr. In some embodiments, after an appropriate equilibration time at least 99% of the nuclide that is complexed to the compound is bound through the chelating ligand selective for ⁸⁹Zr or at least 99% of the nuclide that is complexed to the compound is bound through the chelating ligand selective for a nuclide of pharmaceutical potential other than ⁸⁹Zr. In some embodiments, after an appropriate equilibration time at least 99.9% of the nuclide that is complexed to the compound is bound through the chelating ligand selective for ⁸⁹Zr or at least 99.9% of the nuclide that is complexed to the compound is bound through the chelating ligand selective for a nuclide of pharmaceutical potential other than ⁸⁹Zr.

In some embodiments, wherein the nuclide is a radionuclide, the appropriate equilibration time is less than 10 half lives, 5 half lives, 2.5 half lives, 1 half lives, 0.75 half lives, 0.5 half lives, 0.25 half lives, 0.1 half lives, 0.05 half lives or 0.01 half lives of the radionuclide that one of the chelating ligands is selective for.

In some embodiments, the appropriate equilibration time is 1 s, 5, s, 10 s, 30 s, 1 min, 2 min, 5 min, 10 min, 30 min, 1 h, 2 h, 4 h, 8 h, 16 h or 1 day. Preferably, the appropriate equilibration time is less than 2 h. Preferably, the appropriate equilibration time is less than 3 h.

In some embodiments, the stability of the nuclide chelating ligand complex is such that the stability constant (log β) of the complex is equal to or more than 10, 15, 20, 25, 25.4, 30, 35, 40, 41, 45, 50 or 60.

In some embodiments, the affinity between the nuclide that the chelating ligand is selective for and the affinity between the nuclide and the chelating ligand that the radionuclide is not selective for differs such that the stability constant (log β) of a complex between the nuclide and the two different chelating ligands differ by at least a value of 10, 15, 20, 25, 25.4, 30, 35, 40, 41, 45, 50 or 60. Preferably, the selectivity differs by a value of 25, 25.4, 40 or 41.

In some embodiments, selectivity and/or affinity is assessed in a mixture such that all components are soluble. In some embodiments, selectivity and/or affinity is assessed in a composition suitable for administration. In some embodiments, selectivity and/or affinity is assessed post-administration in a mixture extracted from a subject. In some embodiments, selectivity and/or affinity is assessed in a composition designed to mimic a post-administration mixture extracted from a subject.

In some embodiments, the first chelating ligand has substantially no affinity to binding a metal for which the second chelating ligand is selective for. In some embodiments, the second chelating ligand has substantially no affinity to binding a metal for which the first chelating ligand is selective for.

### First Chelating Ligand

The first chelating ligand is selective for ⁸⁹Zr. In some embodiments, the first chelating ligand is selective for ⁸⁹Zr(IV).

In some embodiments, the first chelating ligand is also selective for a further nuclide of pharmaceutical potential, such as ⁹⁰Nb.

In some embodiments, the first chelating ligand is also selective for one or more further nuclides of pharmaceutical potential other than ⁸⁹Zr.

In some embodiments, the first chelating ligand comprises one or more hydroxamic acid or hydroxypyridone groups. Hydroxamic acid or hydroxypyridone functional groups are a suitable ligand for ⁸⁹Zr.

In some embodiments, the first chelating ligand is hexadentate. In some embodiments, the first chelating ligand is octadentate.

In some embodiments, the first chelating ligand is a radical of desferrioxamine B (DFOB).

In some embodiments, the compound further comprises a moiety to enhance ⁸⁹Zr affinity of the chelating ligand selective for ⁸⁹Zr. Preferably, the presence of the moiety to enhance ⁸⁹Zr affinity of the chelating ligand selective for ⁸⁹Zr in the compound means that substantially all of the ⁸⁹Zr complexed to the compound is octa-coordinated, i.e. eight atoms of the chelating ligand collaborate in complex formation with the atom (the coordination number is 8). Preferably, substantially all of the ⁸⁹Zr complexed to the compound is octa-coordinated through the donor oxygen atoms present in the hydroxamic acid functional groups.

In some embodiments, the moiety to enhance ⁸⁹Zr affinity may be selected from 5-((5-aminopentyl)(hydroxy)amino)-5-oxopentanoic acid (PPH), 5-((2-(2-aminoethoxy)ethyl)(hydroxy)amino)-5-oxopentanoic acid (PPH-^{N}O), 2-(2-((5-aminopentyl)(hydroxy)amino)-2-oxoethoxy)acetic acid (PPH-^{C}O), 2-(2-((2-(2-aminoethoxy)ethyl)(hydroxy)amino)-2-oxoethoxy)acetic acid (PPH-^{N}O^{C}O) 2-((2-((5-aminopentyl)(hydroxy)amino)-2-oxoethyl)thio)acetic acid (PPH-^{C}S) and 2-((2-((2-(2-aminoethoxy)ethyl)(hydroxy)amino)-2-oxoethyl)thio)acetic acid (PPH-^{N}O^{C}S).

In some embodiments of the compound of formula (I), A is

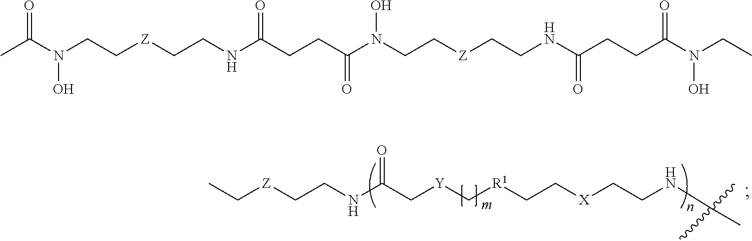

wherein R¹ is

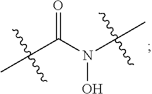 [SMILES: *C(=O)N(*)O]

Y is CH₂, O or S;
X is CH₂, O or S;
each Z is independently selected from CH₂ or O;
n is 0 or 1; and
m is 0 or 1.

In some embodiments, every instance of Z is CH₂.

In some embodiments, every instance of Z is O. Methods of synthesis of compounds where Z is O are known in the art, for instance in WO 2017/096430.

In some embodiments, Y is CH₂ or O.

In some embodiments, X is CH₂ or O.

In some embodiments, n is 0.

In some embodiments, n is 1;
Y is CH₂, O or S;
X is CH₂, O or S; and
m is 0 or 1.

In some embodiments, n is 1;
Y is CH₂ or O,
X is CH₂ or O, and
m is 0 or 1.

In some embodiments, n is 1, Y is CH₂, X is CH₂ and m is 1.

In some embodiments, n is 1, Y is CH₂, X is CH₂ and m is 0.

In some embodiments, n is 1, Y is CH₂, X is O and m is 0.

In some embodiments, n is 1, Y is CH₂, X is O and m is 1.

In some embodiments, n is 1, Y is O, X is CH₂ and m is 1.

In some embodiments, n is 1, Y is O, X is O and m is 1.

In some embodiments, n is 1, Y is S, X is CH₂ and m is 1.

In some embodiments, n is 1, Y is S, X is O and m is 1.

In some embodiments, A is selected from the group consisting of

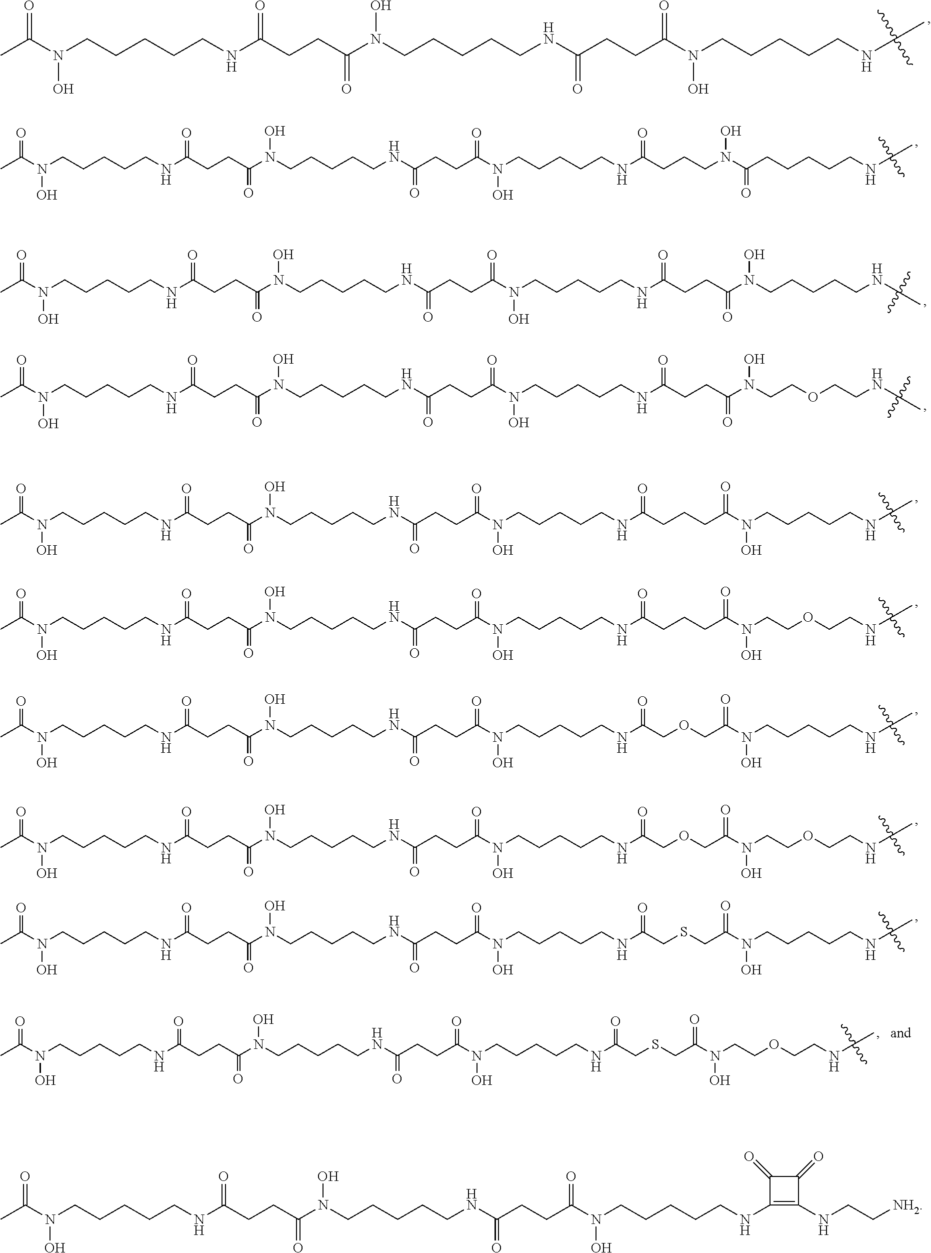

Second chelating Ligand

The second chelating ligand is selective for a nuclide of pharmaceutical potential other than ⁸⁹Zr.

In some embodiments, the second chelating ligand is selective for one or more nuclides of the group consisting of ⁹⁰Y, ¹⁵³Sm, ¹⁶¹Tb, ¹⁶¹Lu, ²¹³Bi and ²²⁵Ac. In some embodiments, the ⁹⁰Y may be ⁹⁰Y(III). In some embodiments, the ¹⁵³Sm may be ¹⁵³Sm(III). In some embodiments, the ¹⁶¹Tb may be ¹⁶¹Tb(III). In some embodiments, the ²¹³Bi may be ²¹³Bi(III). In some embodiments, the ²²⁵Ac may be ²²⁵Ac(III). The second chelating ligand may be selective for a range of trivalent metals, such as known in the literature [Mishiro, K.; Hanaoka, H.; Yamaguchi, A.; Ogawa, K. Radiotheranostics with radiolanthanides: Design, development strategies, and medical applications. Coord. Chem. Rev. 2019, 383, 104-131].

In some embodiments, the second chelating ligand comprises a polyaminocarboxylic acid group. Polyaminocarboxylic acids are suitable chelating ligands for ⁹⁰Y(III), ¹⁵³Sm, ¹⁶¹Tb, ¹⁷⁷Lu(III), ²¹³Bi(III) and ²²⁵Ac(III). Further, polyaminocarboxylic acids are also relatively poor chelators of ⁸⁹Zr, with the formation of these complexes requiring high temperatures (99° C.) and extended reaction times (2 h) to give modest radiochemical yields (65%). These elevated temperatures and extended reaction times are poorly compatible with many functionalities and molecules, including sensitive biomolecules such as antibodies that may be present in the compound for immunological applications.

In some embodiments, the second chelating ligand is a radical of DOTA (1,4,7,10-tetraazacyclododecane-1,4,7,10-tetraacetic acid), DOTAGA (alpha-(2-carboxyethyl)-1,4,7,10-tetraazacyclododecane-1,4,7,10-tetraacetic acid) or NETA (7-[2-[bis(carboxymethyl)amino]ethyl]hexahydro-1H-1,4,7-triazonine-1,4(5H)-diacetic acid).

In some embodiments of the compound of formula (I), B is selected from the group consisting of:

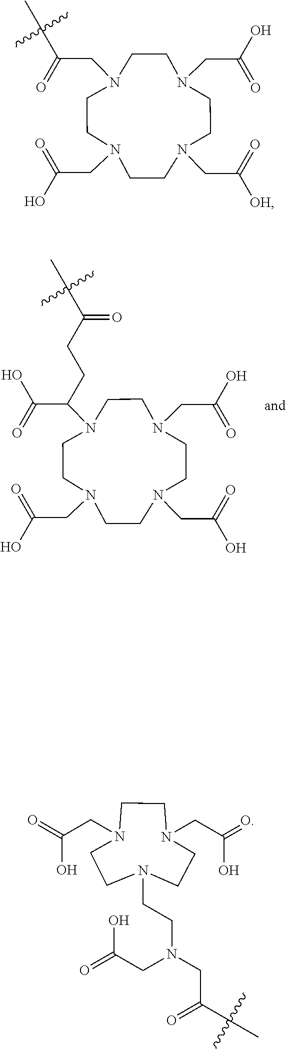 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1 ; *C(=O)CN(CCN1CCN(CC(=O)O)CCN(CC(=O)O)CC1)CC(=O)O]

In some embodiments of the compound of formula (I), B is

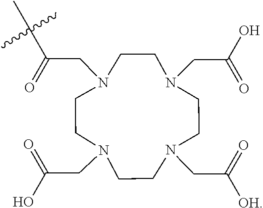 [SMILES: *C(=O)CN1CCN(CC(=O)O)CCN(CC(=O)O)CCN(CC(=O)O)CC1]

### Linker Group

The first and second chelating ligands are linked through a linker group.

The linker group may define any suitable connection between the two chelating ligands.

In some embodiments, the linker group is a covalent bond.

In some embodiments, the linker group may be any suitable diradical species functionalised to form a covalent bond with the first chelating ligand and a covalent bond to the second chelating ligand.

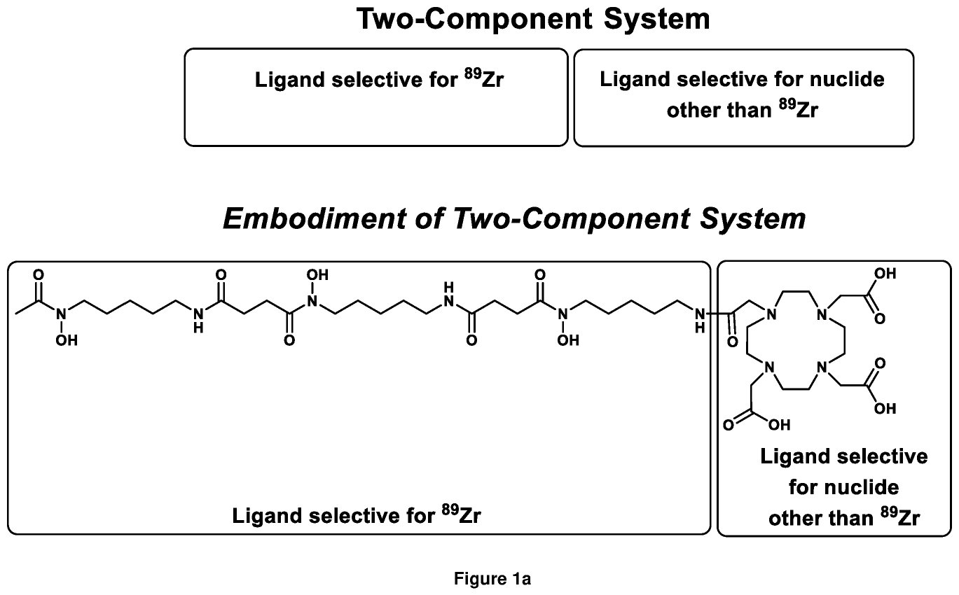

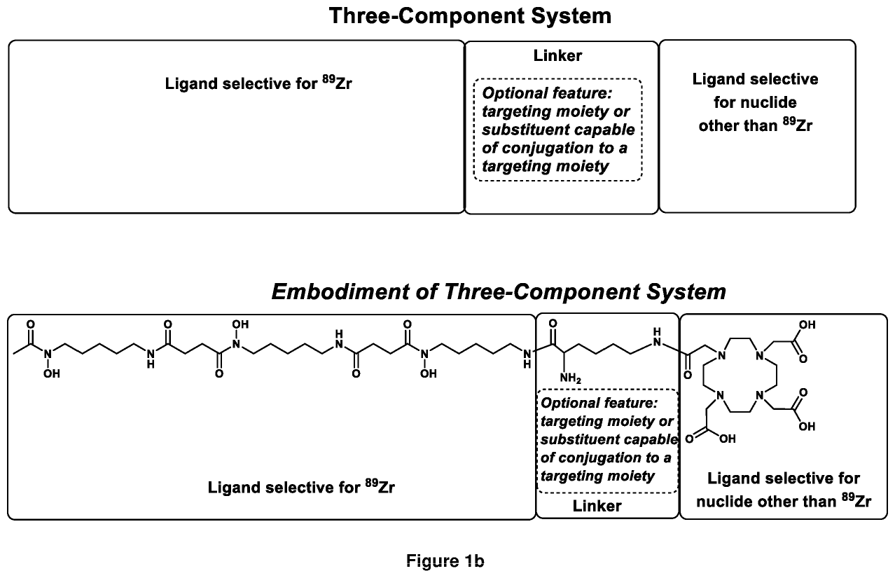

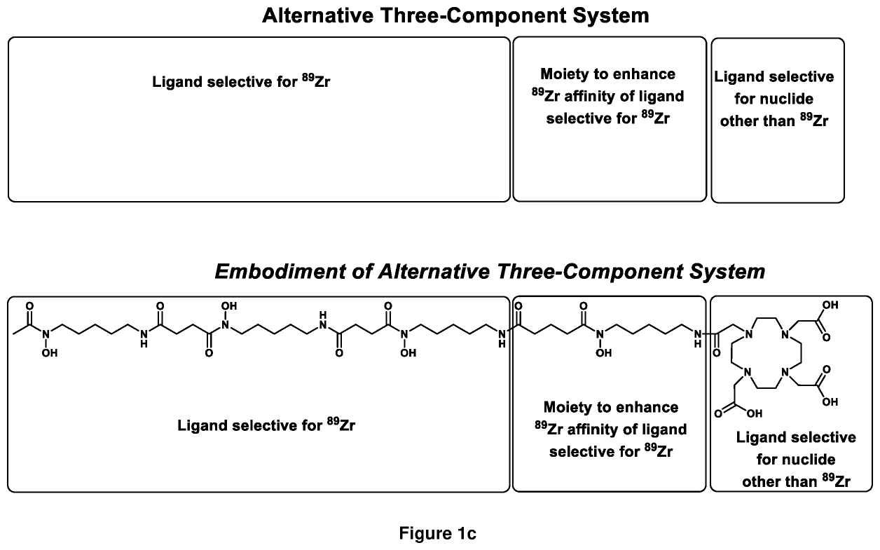

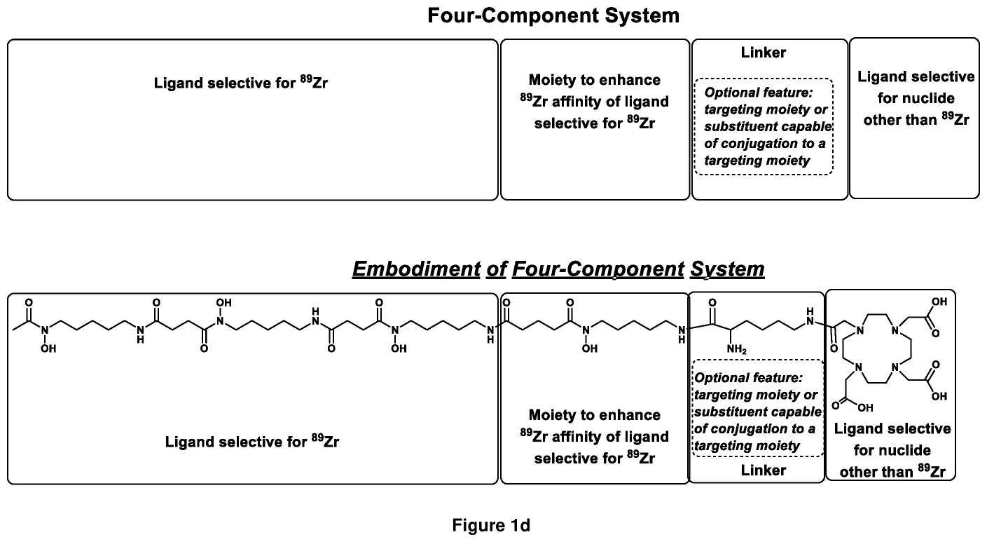

In any linker group, a path from the first chelating group to the second chelating group may be defined by the shortest route, which takes the fewest number of atoms. The linker group may therefore be defined by the shortest linear chain of covalently bonded atoms between the two chelating ligands. For example, in the structure shown in FIG. 1a, the linker group is a covalent bond with zero atoms between the first and second chelating ligands, while in the structure shown in FIG. 1b, the linker group has a shortest route of 7 atoms (including the amide carbonyl covalently linked to the N-atom of the DFOB moiety, and the amide nitrogen atom covalently linked to the DOTA moiety carbonyl.

The shortest chain of the linker group may be up to 30, 25, 20, 15, 10, 9, 8, 7, 6, 5, 4, 3, 21 or 0 atoms. In some embodiments, the shortest chain may be from any of these values to any other value, such as from 1 to 30 atoms or 4 to 10 atoms.

The linker may be a straight-chain of atoms covalently bound to the first chelating ligand and to the second chelating ligand and may comprise any degree of branching or substitution. In some embodiments, the linker group may comprise one or more cyclic structures, which may be selected from optionally substituted cycloalkyl, optionally substituted aryl or optionally substituted heterocyclyl groups, which may optionally be included in a fused, bridged or spirocyclic system.

Suitable linker group atoms include C (carbon), N (nitrogen), O (oxygen) and S (sulphur). Linker group atoms are bound to other atoms such as H (hydrogen) to fulfil the normal rules of valency. The linker group may be saturated or unsaturated.

In some embodiments, the linker group is an optionally substituted C₁₋₂₀ alkyl chain interrupted by one or more functional groups selected from heteroatoms, alkenes, alkynes, cycloalkyl groups, heterocyclyl groups, amides, esters, ketones and a targeting moiety. In some embodiments, the alkyl chain of the linker is interrupted by 10 groups or fewer, 8 groups or fewer, 6 groups or fewer, 4 groups or fewer, 3 groups or fewer, 2 groups or fewer, or 1 group.

In some embodiments the linker group is an oligopeptide comprising 10 or fewer amino acid residues, 8 or fewer amino acid residues, 6 or fewer amino acid residues, 4 or fewer amino acid residues, 3 or fewer amino acid residues, or 2 or fewer amino acid residues. In some embodiments, the linker group comprises a single amino acid residue. In some embodiments, the amino acid residues are selected from the 20 naturally occurring amino acids commonly designated by three letter symbols and also include, 4-hydroxyproline, hydroxylysine, 3-methylhistidine, norvaline, beta-alanine, gamma-aminobutyric acid, citrulline, homocysteine, homoserine and ornithine. In some embodiments, the amino acid residues are selected from the 20 naturally occurring amino acids commonly designated by three letter symbols. Preferably, the linker group comprises lysine, glutamic acid, aspartic acid or combinations thereof. Preferably, the linker comprises lysine. Preferably, the linker comprises glutamic acid. Preferably, the linker comprises aspartic acid.

In some embodiments, the linker is a conjugate of L-glutamic acid. In some embodiments, the linker is a conjugate of D-glutamic acid. In some embodiments, the linker is a conjugate bound to one of the chelating groups through the alpha-carboxylic acid of glutamic acid. In some embodiments, the linker is a conjugate bound to one of the chelating groups through the gamma-carboxylic acid of glutamic acid.

In some embodiments, the linker is a conjugate of L-aspartic acid. In some embodiments, the linker is a conjugate of D-aspartic acid. In some embodiments, the linker is a conjugate bound to one of the chelating groups through the alpha-carboxylic acid of aspartic acid. In some embodiments, the linker is a conjugate bound to one of the chelating groups through the beta-carboxylic acid of aspartic acid.

In some embodiments, the linker is a conjugate of L-lysine. In some embodiments, the linker is a conjugate of D-lysine. In some embodiments, the linker is a conjugate bound to one of the chelating groups through the alpha-amine of lysine. In some embodiments, the linker is a conjugate bound to one of the chelating groups through the epsilon-amine of lysine. In some embodiments, the linker group comprises one or more ethylene glycol repeat units.

The linker is optionally substituted with one or more substituents. Optional substituents include C₁₋₆alkyl, C₂₋₆alkenyl, C₂₋₆alkynyl, C₃₋₈cycloalkyl, hydroxyl, oxo, C₁₋₆alkoxy, aryloxy, C₁₋₆alkoxyaryl, halo, C₁₋₆alkylhalo (such as CF₃), C₁₋₆alkoxyhalo (such as OCF₃), carboxyl, esters, cyano, nitro, amino, substituted amino, disubstituted amino, acyl, ketones, substituted ketones, amides, aminoacyl, substituted amides, disubstituted amides, thiol, C₁₋₆alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arylC₁₋₆alkyl, heterocyclyl and heteroaryl wherein each alkyl, alkenyl, alkynyl, cycloalkyl, aryl and heterocyclyl and groups containing them may be further optionally substituted. Optional substituents may be further substituted with 1, 2, 3, 4 or more groups, preferably 1, 2 or 3, more preferably 1 or 2 groups selected from the group provided above.

In another aspect, the invention provides a compound of formula (II)

Ch¹-L-Ch² (II)
wherein
Ch¹ comprises a radical of DFOB;
Ch² comprises a radical of DOTA; and
L is a linker group.

Ch¹ may be any radical of DFOB suitable for forming a covalent bond with the linker group. Ch¹ may be any radical of DFOB described herein for the first chelating ligand. In some embodiments, Ch¹ comprises a moiety that enhances the affinity of DFOB to ⁸⁹Zr. For example, in some embodiments, Ch¹ has the following partial structure:

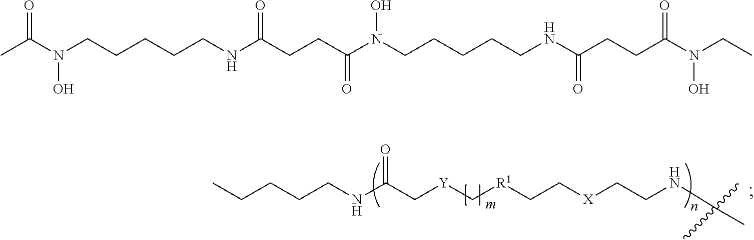

wherein R¹ is

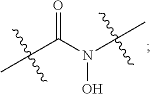 [SMILES: *C(=O)N(*)O]

Y is CH₂, O or S;
X is CH₂, O or S;
n is 0 or 1; and
m is 0 or 1.

In some embodiments, m is 0 and Y is CH₂. In some embodiments, m is 1 and Y is CH₂, O or S.

Ch² may have the structure of any radical of DOTA suitable for forming a covalent bond with the linker group. Ch¹ may be any radical of DOTA described herein for the second chelating ligand. For example, Ch² may have the partial structure:

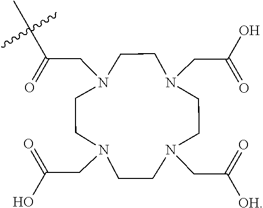

Ch² may have the structure of any radical of DOTAGA suitable for forming a covalent bond with the linker group. Ch¹ may be any radical of DOTAGA described herein for the second chelating ligand. For example, Ch² may have the partial structure:

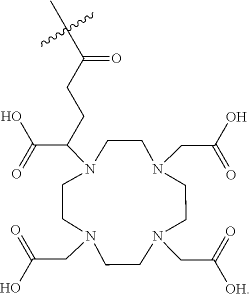

Ch² may have the structure of any radical of NETA suitable for forming a covalent bond with the linker group. Ch¹ may be any radical of NETA described herein for the second chelating ligand. For example, Ch² may have the partial structure:

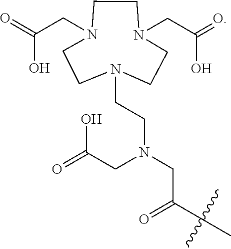

The linker group of the compound of formula (II) may be the same as any covalent linker group described herein.

### Targeting Moiety

In some embodiments, the compound of the invention comprises a targeting moiety or a group capable of conjugation to a targeting moiety. The targeting moiety or group capable of conjugation to a targeting moiety may typically be incorporated into the linker group. In some embodiments, the linker group comprises a spacer moiety extending from the linker to the targeting moiety or group capable of conjugation to the targeting moiety. The spacer may extend from the linker by 1-20 atoms (longest linear chain) typically selected from C, O and N. In some embodiments, the spacer moiety is an optionally substituted C₁₋₂₀alkyl group optionally interrupted by 1-10 functional groups selected from the group consisting of ethers, hydroxyl groups, amines and carboxylic acids, and combinations thereof. Advantageously the spacer moiety may comprise one or more of these hydrophilic moieties to aid in increasing the compounds solubility in aqueous environments. In some embodiments, the spacer moiety comprises one or more ethers so as to form a polyethylene glycol moiety within the spacer. The polyethylene glycol may define a portion of the spacer moiety or the spacer moiety may consist of the polyethylene glycol group terminating in the targeting moiety or the functional group capable of conjugation with the targeting moiety. The polyethylene glycol group may comprise 2-20 —(CH₂)₂O— repeating units, preferably 2-10 units. However, in some embodiments, the first and/or second chelating ligands may be functionalised to include the targeting moiety or group capable of conjugation to a targeting moiety. The group capable or conjugation to a targeting moiety could instead be conjugated to a solid support.

A targeting moiety directs the compound to a targeted tissue, organ, receptor or other biologically expressed composition. In some embodiments, the targeting moiety is selective or specific for the targeted organ or tissue. Suitable targeting moieties and groups capable of conjugation to the targeting moieties that may be included in the compounds of the invention are described in WO2017161356 (Waddas) and WO2015140212 (Gasser).

In some embodiments the targeting moiety is suitable for immuno-PET imaging. In some embodiments the targeting moiety is suitable for treatment of a neoplastic disorder.

Broadly, the targeting moiety may be an antibody, an amino acid, a nucleoside, a nucleotide, an aptamer, a protein, an antigen, a peptide, a nucleic acid, an enzyme, a lipid, an albumin, a cell, a carbohydrate, a vitamin, a hormone, a nanoparticle, an inorganic support, a polymer, a single molecule or a drug. Specific examples of the targeting moiety include: steroid hormones for the treatment of breast and prostate lesions; somatostatin, bombesin, CCK, and neurotensin receptor binding molecules for the treatment of neuroendocrine tumors; CCK receptor binding molecules for the treatment of lung cancer; ST receptor and carcinoembryonic antigen (CEA) binding molecules for the treatment of colorectal cancer; dihydroxyindolecarboxylic acid and other melanin producing biosynthetic intermediates for the treatment of melanoma; integrin receptor; fibroblast activation protein alpha (FAP) and atherosclerotic plaque binding molecules for the treatment of vascular diseases; and amyloid plaque binding molecules for the treatment of brain lesions. Examples of the targeting moiety also include synthetic polymers such as polyaminoacids, polyols, polyamines, polyacids, oligonucleotides, aborols, dendrimers, and aptamers.

In some embodiments, the present invention relates to the incorporation of a targeting moiety that may be selected from among nanoparticles, antibodies (e.g., Technetium (99m Tc) fanolesomab (NeutroSpect®), girentuximab (Rencarex®), ibritumomab tiuxetan (Zevalin®) and adalimumab (Herceptin®)), proteins (e.g., TCII, HSA, annexin and Hb), peptides (e.g., octreotide, bombesin, neurotensin and angiotensin), nitrogen-containing simple or complex carbohydrates (e.g., glucosamine and glucose), nitrogen-containing vitamins (e.g., vitamin A, B₁ B₂, B₁₂, C, D₂, D₃, E, H and K), nitrogen-containing hormones (e.g., estradiol, progesterone and testosterone), nitrogen-containing active pharmaceuticals (e.g., celecoxib or other nitrogen-containing NSAIDs, AMD3100, CXCR4 and CCR5 antagonists) and nitrogen-containing steroids. In some embodiments, the present invention relates to the incorporation of the targeting moiety girentuximab.

In some embodiments, the compound of the present invention may be substituted with a targeting moiety or a substituent capable of conjugation to a targeting moiety.

In some embodiments, the present invention may include conjugates of a compound, complex, pharmaceutical agent or composition of the invention having multiple targeting moieties. For example, to increase specificity for a particular target tissue, organ receptor or other biologically expressed composition, multiple bioactive substances or chemically active substances may be utilized. In such instances, the targeting moieties may be the same or different. For example, a single conjugate may possess multiple antibodies or antibody fragments, which are directed against a desired antigen or hapten. Typically, the antibodies used in the conjugate are monoclonal antibodies or antibody fragments that are directed against a desired antigen or hapten. Thus, for example, the conjugate may include two or more monoclonal antibodies having specificity for a desired epitope and thereby increasing concentration of the conjugate at the desired site. Similarly, and independently, a conjugate may include two or more different bioactive substances or chemically active substances each of which is targeted to a different site on the same target tissue or organ. By utilizing multiple targeting moieties in this manner, the conjugate is advantageously concentrated at several areas of the target tissue or organ, potentially increasing the effectiveness of therapeutic treatment or diagnosis. In an embodiment, the targeting moiety may comprise peptides, proteins, peptide or protein dimers, trimers and multimers. Further, the conjugate may have a ratio of bioactive substances or chemically active substances, designed to concentrate the conjugate at a target tissue or organ and optimally achieve the desired therapeutic and/or diagnostic results while minimizing non-target deposition. Alternatively, and/or additionally, the present invention relates to a two-step, pre-targeting strategy.

It is contemplated and therefore within the scope of the invention that the compounds, complexes, pharmaceutical agents and compositions of the present invention can be modified to target specific receptors or cancer cells or can be modified so that they can survive various in vivo environments. In a variation, the conjugates, compositions, and methods of the present invention can be used against solid tumors, cell lines, and cell line tissue that demonstrate upregulated nucleotide excision repair and other upregulated resistance mechanisms.

In some embodiments, the compound, complex, pharmaceutical agent or composition of the invention is conjugated to one or more receptor-specific molecules comprising an antibody, an oligopeptide, a polypeptide, or one or more small molecule compounds for targeting cancer-type specific receptors and/or receptors overexpressed in certain cancer types.

In some embodiments, the targeting moiety or the substituent capable of conjugation to a targeting moiety is selected from one or more members of one or more of Groups a), b), c), d) and e);
wherein
Group a) consists of: an OH, NH₂, SH, COOH, CHO, N₃, SCN, CH₂X (X=Cl, Br, I), an activated ester (such as N-hydroxysuccinimide, tetra- or pentafluoro phenol derivatives), an ene-one-system (such as an alpha,beta-unsaturated carbonyl, or a Michael acceptor system, such as maleimide), a diene or dienophile suitable for the Diels-Alder reaction, an alkene, and an alkyne;
Group b) consists of: a first click moiety capable of selectively forming a covalent bond with a second click moiety under reaction conditions not leading to a covalent reaction of the first or second click moiety with natural occurring polypeptides, in particular with proteins;
Group c) consists of: an antibody, an oligopeptide, a polypeptide, a polynucleotide, a liposome, a polymerosome, a phospholipid, a vitamin, a monosaccharide, an oligosaccharide, a nanoparticle, or a drug-like molecule having a molecular weight less than (<) 3000 U, or a moiety that specifically binds to a target site on cells and/or tissues with an association constant of lower than (<) 10⁻⁶ mol/L, <10⁻⁷ mol/L, <10⁻⁸ mol/L or <10⁻⁹ mol/L,
Group d) consists of: an antibody, an oligopeptide, a polypeptide or protein, a polynucleotide, a liposome, a polymerosome, a phospholipid, a vitamin, a monosaccharide, an oligosaccharide, a nanoparticle, or a drug-like molecule having a molecular mass less than (<) 3000 U, any of which is selective for a disease specific ligand, a cell specific ligand or a tissue specific ligand; and
Group e) consists of: a solid support.

In some embodiments, the targeting moiety or the substituent capable of conjugation to a targeting moiety is selected from the group consisting of an OH, NH₂, SH, COOH, N₃, SCN, an activated ester, a maleimide, and an alkyne. Preferably, OH, NH₂, SH, N₃, a maleimide, and an alkyne. More preferably, OH, NH₂, N₃, and an alkyne. More preferably, OH and NH₂. Most preferably, NH₂.

In some embodiments, the targeting moiety or the substituent capable of conjugation to a targeting moiety comprises, or is, one partner of two partners forming a so-called click reaction couple. In such embodiments, the targeting moiety or the substituent capable of conjugation to a targeting moiety is a first click moiety capable of forming a covalent bond selectively with a second click moiety under reaction conditions not leading to a covalent reaction of the first or second moieties with natural occurring polypeptides, in particular with proteins. The click reactive groups are meant to conjugate the chelating ligand to molecules of interest and at the same time provide the possibility of novel pre-targeting approaches. In some embodiments, the targeting moiety or the substituent capable of conjugation to a targeting moiety is selected from the group consisting of an azide, an alkyne, a tetrazine, a cyclooctyne and a trans-cyclooctene. Suitable click reaction partners are well known in the art.

The compounds of the invention may be provided in the form of a pharmaceutically acceptable salt, solvate, tautomer, stereoisomer, polymorph and/or prodrug.

The term “pharmaceutically acceptable” may be used to describe any salt, solvate, tautomer, stereoisomer, polymorph and/or prodrug thereof, or any other compound which upon administration to a subject, is capable of providing (directly or indirectly) a compound of the invention or an active metabolite or residue thereof and typically that is not unacceptably deleterious to the subject.

The salts of the compounds of the invention are preferably pharmaceutically acceptable, but it will be appreciated that non-pharmaceutically acceptable salts also fall within the scope of the present disclosure, for example, as these may be useful as intermediates in the preparation of pharmaceutically acceptable salts or in methods not requiring administration to a subject.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

In the case of compounds that are solids, it will be understood by those skilled in the art that the inventive compounds, agents and salts may exist in different crystalline or polymorphic forms, all of which are intended to be within the scope of the present invention and specified formulae.

The invention includes all crystalline forms of a compound of the invention including anhydrous crystalline forms, hydrates, solvates and mixed solvates. If any of these crystalline forms demonstrates polymorphism, all polymorphs are within the scope of this invention.

The compounds of the invention are intended to cover, where applicable, solvated as well as unsolvated forms of the compounds. Thus, the compounds of the invention include compounds having the indicated structures, including the hydrated or solvated forms, as well as the non-hydrated and non-solvated forms.

The compounds of the invention or salts, tautomers, polymorphs or prodrugs thereof may be provided in the form of solvates. Solvates contain either stoichiometric or non-stoichiometric amounts of a solvent, and may be formed during the process of crystallization with pharmaceutically acceptable solvents such as water, alcohols such as methanol, ethanol or isopropyl alcohol, DMSO, acetonitrile, dimethyl formamide (DMF), acetic acid, and the like with the solvate forming part of the crystal lattice by either non-covalent binding or by occupying a hole in the crystal lattice. Hydrates are formed when the solvent is water, alcoholates are formed when the solvent is alcohol. Solvates of the compounds of the present invention can be conveniently prepared or formed during the processes described herein. In general, the solvated forms are considered equivalent to the unsolvated forms for the purposes of the invention.

Basic nitrogen-containing groups may be quarternised with such agents as C₁₋₆alkyl halide, such as methyl, ethyl, propyl, and butyl chlorides, bromides and iodides; dialkyl sulfates like dimethyl and diethyl sulfate; and others.

The compound of the invention or salts, tautomers, solvates and/or prodrugs thereof that form crystalline solids may demonstrate polymorphism. All polymorphic forms of the compounds, salts, tautomers, solvates and/or prodrugs are within the scope of the invention.

The compound of the invention may demonstrate tautomerism. Tautomers are two interchangeable forms of a molecule that typically exist within an equilibrium. Any tautomers of the compounds of the invention are to be understood as being within the scope of the invention.

A “prodrug” is a compound that may not fully satisfy the structural requirements of the compounds provided herein, but is modified in vivo, following administration to a subject or patient, to produce a compound of the invention provided herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of compounds of the invention. The amino acid residues include the 20 naturally occurring amino acids commonly designated by three letter symbols and also include, 4-hydroxyproline, hydroxylysine, 3-methylhistidine, norvaline, beta-alanine, gamma-aminobutyric acid, citrulline, homocysteine, homoserine, omithine and methionine sulfone.

The compound of the invention may contain one or more stereocentres. All stereoisomers of the compound of the invention are within the scope of the invention. Stereoisomers include enantiomers, diastereomers, geometric isomers (E and Z olephinic forms and cis and trans substitution patterns) and atropisomers. In some embodiments, the compound is a stereoisomerically enriched form of the compound of the invention at any stereocentre. The compound may be enriched in one stereoisomer over another by at least about 60, 70, 80, 90, 95, 98 or 99%.

The compound of the invention may be isotopically enriched with one or more of the isotopes of the atoms present in the compound. For example, the compound may be enriched with one or more of the following minor isotopes: ²H, ³H, ¹³C, ¹⁴C, ¹⁵N and/or ¹⁷O. An isotope may be considered enriched when its abundance is greater than its natural abundance.

### Complexes

In a further aspect, there is provided a complex comprising a compound of the present invention and a metal.

In some embodiments, the metal(s) is a radionuclide(s) of pharmaceutical potential. In some embodiments the metal(s) is a naturally occurring, non-toxic, isotope of the metal.

In some embodiments, the complex comprises one metal. In some embodiments, the complex comprises two different metals, preferably wherein one metal is of therapeutic potential and the second metal is of diagnostic potential.

In one or more aspects and embodiments of the invention, the invention also relates to the compound of the invention in a complex with one metal or two different metals. In some embodiments, one metal is coordinatively bound to the binding moieties of the first chelating ligand in the compound. In some embodiments, one metal is coordinatively bound to the binding moieties of the second chelating ligand in the compound. In some embodiments, one metal that is different to the metal bound to the first chelating ligand, is coordinatively bound to the binding moieties of the second chelating ligand. In some embodiments, the complex comprises two different metals. In some embodiments, one or both of the metals are ions. In some embodiments, one or both of the two different metal ions is octa-coordinated, eg. eight atoms of the first chelating ligand collaborate in complex formation with the metal (the coordination number is 8), particularly where the metal is Zr or more particularly where the metal is ⁸⁹Zr.

In some embodiments, the metal ion of the first chelating ligand is hexa-coordinated, i.e. six atoms of the first chelating ligand collaborate in complex formation with the metal ion, particularly where the metal is Zr or more particularly where the metal is ⁸⁹Zr. In some embodiments, the metal atom of the first chelating ligand is tetra-coordinated. In some embodiments, the metal atom of the first chelating ligand is penta-coordinated.

In some embodiments, the metal ion of the second chelating ligand is octa-coordinated, i.e. eight atoms of the second chelating ligand collaborate in complex formation with the metal ion (the coordination number is 8), particularly a radionuclide of pharmaceutical potential other than ⁸⁹Zr. In some embodiments, the metal ion of the second chelating ligand is hepta-coordinated, i.e. seven atoms of the second chelating ligand collaborate in complex formation with the metal ion (the coordination number is 7), particularly a radionuclide of pharmaceutical potential other than ⁸⁹Zr. In some embodiments, the metal ion of the second chelating ligand is hexa-coordinated. In some embodiments, the metal ion of the second chelating ligand is penta-coordinated. In some embodiments, the metal ion of the second chelating ligand is tetra-coordinated.

Even if a metal does not require all coordination sites provided by the chelating ligand system, the chelating ligand will increase the stability of the complex simply by providing higher “concentration” of coordinating groups in the near vicinity of the metal which will protect it from trans-chelation.

In some embodiments, the metal(s) of the complex has the oxidation number +1, +2, +3, +4, +5, +6, or +7. In some embodiments, the metal(s) of the complex has the oxidation number +3, +4, +5 or +6. In some embodiments, the metal(s) of the complex has a coordination number from 4 to 8.

In some embodiments, the metal(s) is in any one of the groups 3, 4, 6, 7, 9, 10, 11, 13, 15, the lanthanide group of elements, or the actinide group of elements of the periodic table of the elements. Groups are assigned in accordance to current IUPAC practice, older designations refer to the “scandium group” (IIIA) for group 3, “titanium group” (IVA) for group 4, “actinides group” for group 6, “manganese group” for group 7, “lanthanides group” comprising group, 9, 10, and 11, “boron group” for group 13, and “nitrogen group” for group 15.

In some embodiments, the metal(s) is a metallic radionuclide. A radionuclide, or a radioactive nuclide, is an atom with an unstable nucleus, which undergoes radioactive decay, resulting in the emission of gamma ray(s) or subatomic particles such as positrons, alpha or beta particles, or Auger electrons. These emissions constitute ionizing radiation. Radionuclides occur naturally or can be produced artificially. Radionuclides are often referred to as radioactive isotopes or radioisotopes.

In some embodiments, the metal(s) is selected from ⁸⁹Zr (diagnostic), ⁹⁰Nb (diagnostic), ⁹⁰Y (therapeutic), ¹⁵³Sm (therapeutic), ¹⁶¹Tb (therapeutic), ¹⁷⁷Lu (therapeutic), ²¹³Bi (therapeutic) and ²²⁵Ac (therapeutic). In some embodiments, the metal(s) is selected from ⁸⁹Zr and ⁹⁰Nb. In some embodiments, the metal(s) is selected from ⁹⁰Y, ¹⁵³Sm, ¹⁶¹Tb, ¹⁷⁷Lu, ²¹³Bi and ²²⁵Ac.

In some embodiments wherein the complex comprises two different metals it is preferred that one of the metals is ⁸⁹Zr and the second metal is ²²⁵Ac or ¹⁷⁷Lu, preferably ¹⁷⁷Lu.

Typically, a metal coordinated to the first chelating ligand is a metal having substantially no affinity to binding a polyaminocarboxylic acid-type ligand, such as DOTA, DOTAGA or NETA. In some embodiments, the complex comprises a metal coordinated to the first chelating ligand selected from Zr (eg ⁸⁹Zr) and Nb (eg ⁹⁰Nb), preferably Zr.

Typically, a metal coordinated to the second chelating ligand is a metal having substantially no affinity to binding a poly-hydroxamic-type ligand, such as DFOB. In some embodiments, the complex comprises a metal coordinated to the second chelating ligand selected from Y (eg ⁹⁰Y), Sm (eg ¹⁵³Sm), Tb (eg ¹⁶¹Tb), Lu (eg ¹⁷⁷Lu), Bi (eg ²¹³Bi) and Ac (eg ²²⁵Ac), preferably Lu.

In some embodiments, wherein the complex comprises two different metals, it is preferred that one of the metals is a radionuclide and the other is a naturally occurring, non-toxic isotope of a metal. In some embodiments, one radionuclide is coordinatively bound to the binding moieties of the first chelating ligand in the compound and one naturally occurring, non-toxic isotope of a metal is coordinatively bound to the binding moieties of the second chelating ligand in the compound. In some embodiments, one naturally occurring, non-toxic isotope of a metal is coordinatively bound to the binding moieties of the first chelating ligand in the compound and one radionuclide is coordinatively bound to the binding moieties of the second chelating ligand in the compound. In some embodiments the metal coordinatively bound to the first chelating ligand is ⁸⁹Zr and the metal bound to the second chelating ligand is the non-toxic, natural Lu(III), ^{nat}Lu. In some embodiments the metal coordinatively bound to the first chelating ligand is the non-toxic, natural Zr(IV), ^{nat}Zr and the metal bound to the second chelating ligand is ¹⁷⁷Lu. A pair of different metals in any combination of natural or radionuclide form (^{nat}Zr(IV) and ^{nat}Lu(III), or ⁸⁹Zr and ¹⁷⁷Lu, or ⁸⁹Zr and ^{nat}Lu(III), or ^{nat}Zr(IV) and ¹⁷⁷Lu) both bound to one compound, where ^{nat}Zr(IV) or ⁸⁹Zr is bound to the first chelating ligand, and ^{nat}Lu(III) or ¹⁷⁷Lu is bound to the second chelating ligand, will have the same pharmacokinetic and biodistribution properties, which is useful for scouting procedures. This example would be useful, since ^{nat}Zr(IV) and ^{nat}Lu(III) are non-toxic in humans.

### Methods of Complexation

In another aspect of the invention, there is provided a method of producing a complex of the invention.

In some embodiments, the compound or a composition comprising the compound is complexed with a metal. Preferably, the metal is a radionuclide. In some embodiments, the compound or a composition comprising the compound is complexed with two different metals. Preferably, one or both of the different metals is a radionuclide. Any method of complexation known to those of ordinary skill in the art can be used to complex any of the compounds or compositions of the present invention.

In some embodiments, the compound or a composition comprising the compound is complexed with a single metal. In some embodiments, the compound or a composition comprising the compound is complexed with two different metals.

In some embodiments, the compound or composition is dissolved in water and a solution of a radionuclide(s) such as ⁸⁹Zr(IV) and/or ¹⁷⁷Lu(III) is added. In some embodiments, the radionuclide(s) added is a radionuclide salt such as ⁸⁹Zr(IV)(acac)₄ and/or ¹⁷⁷Lu(III)Cl₃.

In some embodiments, a mixture comprising the compound or composition and a radionuclide(s) is heated upon mixing of the compound and the radionuclide. In some embodiments, the mixture is heated to more than about 25° C., more than about 30° C., more than about 35° C., more than about 37° C., more than about 40° C., more than about 50° C., more than about 60° C., more than about 70° C., or more than about 80° C. In some embodiments, the mixture is heated to about 37° C. In some embodiments, the mixture is heated to less than about 80° C., less than about 70° C., less than about 60° C., less than about 50° C., less than about 40° C., less than about 37° C., less than about 35° C., or less than about 30° C.

In some embodiments, a mixture comprising the compound and a radionuclide(s) is left at ambient temperature (about 25° C.) upon mixing of the compound and the radionuclide(s).

In some embodiments, a mixture comprising the compound and radionuclide(s) comprises thermally sensitive functionalities, such as in an antibody, affibody, protein, peptide or equivalent. In these embodiments the mixture is preferably left at about 37° C., at less than about 37° C. or at ambient temperature (about 25° C.) upon mixing of the compound and the radionuclide(s).

In some embodiments, a mixture comprising the compound and a radionuclide(s) is chilled upon mixing of the compound and the radionuclide(s). In some embodiments, the mixture is chilled to less than about 25° C., less than about 20° C., less than about 15° C., or less than about 10° C.

In some embodiments, one or more of a solution comprising the compound and a solution comprising the radionuclide(s) is buffered.

Any method known to those of ordinary skill in the art can be used to measure radiochemical purity. For example, it may be measured using radio-thin layer chromatography (r-TLC) with a suitable solvent system. The solvent system will depend on the particular compound tested. For example, conditions for r-TLC are described in Australian patent application no. 2011200132 may be adapted for the present compounds.

Any method known to those of ordinary skill in the art can be used to isolate the radiolabelled compound from solution. For example, using resins to remove unwanted components, with the solution containing the purified radiolabelled compound, evaporating to dryness, and then later reconstituting in water or buffered water for use. In some embodiments, the radiolabelled compound is isolated by high-performance liquid chromatography (HPLC). In some embodiments, the radiolabelled compound is isolated by solvent extraction or trituration.

### Pharmaceutical Agent

In a further aspect, there is provided a pharmaceutical agent comprising a complex of a compound of the invention and one nuclide of pharmaceutical potential.

In some embodiments, the pharmaceutical agent comprises two different nuclides of pharmaceutical potential.

In some embodiments, the pharmaceutical agent comprises a complex of a compound of the invention and one nuclide of pharmaceutical potential. In some embodiments, the pharmaceutical agent comprises a complex of a compound of the invention and two different nuclides of pharmaceutical potential, preferably wherein one nuclide is of therapeutic potential and the second nuclide is of diagnostic potential.

In some embodiments, the pharmaceutical agent is a therapeutic agent, wherein at least one of the nuclides is of therapeutic potential. In some embodiments, at least one of the nuclides is a radionuclide of therapeutic potential. Preferably, at least one of the radionuclides of therapeutic potential is selected from the group consisting of ⁹⁰Y, ¹⁵³Sm, ¹⁶¹Tb, ¹⁷⁷Lu, ²¹³Bi and ²²⁵Ac. More preferably, at least one of the radionuclides of therapeutic potential is selected from the group consisting of ¹⁷⁷Lu and ²²⁵Ac. Even more preferably, the at least one of the radionuclides of therapeutic potential is ¹⁷⁷Lu.

In some embodiments, the pharmaceutical agent is a diagnostic agent, wherein at least one of the nuclides is of diagnostic potential. In some embodiments, at least one of the nuclides is a radionuclide of diagnostic potential. Preferably, at least one of the radionuclides of diagnostic potential is selected from the group consisting of ⁸⁹Zr and ⁹⁰Nb. More preferably, at least one of the radionuclides of diagnostic potential is selected from the group consisting of ⁸⁹Zr.

In some embodiments, the pharmaceutical agent is a theranostic agent, wherein the theranostic agent comprises a complex of a compound of the invention and a nuclide of therapeutic potential and a different nuclide of diagnostic potential.

In some embodiments, the pharmaceutical agent is a prognostic agent, wherein the nuclide is of prognostic potential.

As described herein, the compound, complex, therapeutic agent or composition of the invention may be used variously as a therapeutic agent, a diagnostic agent, theranostic agent or a prognostic agent.

### Compositions

In a further aspect, there is provided a composition comprising a compound, complex or pharmaceutical agent of the present invention and a pharmaceutically acceptable excipient.

The pharmaceutically acceptable excipient is typically a substance which is pharmaceutically inert, confers a suitable consistency or form to the composition, and does not diminish the therapeutic or diagnostic efficacy of the compound, complex or pharmaceutical agent. The excipient is generally considered to be “pharmaceutically acceptable” if it does not produce an unacceptably adverse, allergic or other untoward reaction when administered to the subject. The term ‘excipient’ includes carrier and diluent.

The selection of the pharmaceutically acceptable excipient tends, at least in part, to be a function of the desired route of administration. In general, compositions of the present disclosure can be formulated for any route of administration so long as the target tissue is available via that route. Compositions may be formulated from compounds, complexes or pharmaceutical agents according to the invention for any appropriate route of administration including but not limited to, for example, parenteral (including subcutaneous, intraperitoneal, intradermal, intravascular (for example, intravenous), intramuscular, spinal, intracranial, intrathecal, intraocular, periocular, intraorbital, intrasynovial and intraperitoneal injection, intracisternal injection as well as any other similar injection or infusion techniques), infusion or implantation techniques (e.g., as sterile injectable aqueous or non-aqueous solutions or suspensions).

Examples of components are described in Martindale—The Extra Pharmacopoeia (Pharmaceutical Press, London 1993), and Remington: The Science and Practice of Pharmacy, 21st Ed., 2005, Lippincott Williams & Wilkins. All methods include the step of bringing the active ingredient, for example a compound, pharmaceutical agent or complex of the invention into association with the pharmaceutically acceptable excipient which constitutes one or more accessory ingredients. In general, the compositions are prepared by uniformly and intimately bringing the active ingredient, for example a compound, pharmaceutical agent or complex of the invention into association with a dissolved excipient or a liquid excipient or both. In the composition the active object compound, pharmaceutical agent or complex is included in an amount sufficient to produce the desired effect. In some embodiments, the composition is formulated for intravenous use.

In some embodiments, the compositions of the present invention may be used as an injectable. The composition intended for injection may be prepared according to any known method, and such compositions may contain one or more agents selected from the group consisting of solvents, co-solvents, solubilizing agents, wetting agents, suspending agents, emulsifying agents, thickening agents, chelating agents, antioxidants, reducing agents, antimicrobial preservatives, buffers, pH adjusting agents, bulking agents, protectants, tonicity adjusters, and special additives. Moreover, other non-toxic pharmaceutically-acceptable excipients which are suitable for the manufacture of an injectable may be used.

Aqueous suspensions may contain the active compounds in an admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycethanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more coloring agents.

The pharmaceutical compositions may be in the form of a sterile injectable aqueous or oleaginous suspension. This suspension may be formulated according to the known methods using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, sterile water for injection (SWFI), Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of an injectable.

In the context of this specification the term “administering” and variations of that term including “administer” and “administration”, includes contacting, applying, delivering or providing a compound, pharmaceutical agent, complex or composition of the invention to an organism, or a surface by any appropriate means.

For the treatment or diagnosis of a disease or a disorder such as a neoplastic disorder, the dose of the biologically active compound, pharmaceutical agent or complex according to the invention may vary within wide limits and may be adjusted to individual requirements. Active compounds, pharmaceutical agents or complexes according to the present invention are generally administered in a therapeutically or diagnostically effective amount. The regular dose may be administered as a single dose or in a plurality of doses. The amount of active ingredient that may be combined with the excipient materials to produce a single dosage form will vary depending upon the subject treated and the particular mode of administration.

It will be understood, however, that the specific dose level for any particular subject will depend upon a variety of factors including the activity of the specific compound, pharmaceutical agent or complex employed, the age, body weight, general health, sex and diet of the subject, time of administration, route of administration, and rate of excretion, drug combination (i.e. other drugs being used to treat or diagnose the subject), and the severity of the particular disorder undergoing therapy. Such treatments may be administered as often as necessary and for the period of time judged necessary by the treating physician. A person skilled in the art will appreciate that the dosage regime or therapeutically or diagnostically effective amount of the compound, pharmaceutical agent or complex of the invention to be administered may need to be optimized for each individual. It will also be appreciated that different dosages may be required for treating or diagnosing different disorders.

The terms “treating”, “treatment” and “therapy” are used herein to refer to curative therapy and preventative therapy. Thus, in the context of the present disclosure the term “treating” encompasses curing, ameliorating or tempering the severity of a disease or a disorder such as a neoplastic disorder and/or associated diseases or their symptoms.

“Preventing” or “prevention” means preventing the occurrence of a disease or a disorder such as a neoplastic disorder or tempering the severity of the neoplastic disorder if it develops subsequent to the administration of the compounds or pharmaceutical compositions of the present invention.

“Subject” includes any human or non-human animal. Thus, in addition to being useful for human treatment, the compounds of the present invention may also be useful for veterinary treatment of mammals, including companion animals and farm animals, such as, but not limited to dogs, cats, horses, cows, sheep, and pigs.

The compounds, pharmaceutical agents or complexes of the present invention may be administered along with an excipient as described above.

The pharmaceutical agent of the invention may be one or more of a radiolabelled scintigraphic or PET imaging agent. Radiolabeled scintigraphic or PET imaging agents having a suitable amount of radioactivity are also provided by the present disclosure. In forming diagnostic radioactive complexes, it is generally preferred to form radioactive complexes in solutions containing radioactivity at concentrations of about 0.01 millicurie (mCi) to 100 mCi per mL. Generally, the unit dose to be administered has a radioactivity of about 0.01 mCi to about 100 mCi, specifically about 1 mCi to about 30 mCi. The volume of the solution to be injected at unit dosage is from about 0.01 mL to about 10 mL. The amount of the radiolabeled conjugate appropriate for administration is dependent upon the distribution profile of the chosen conjugate in the sense that a rapidly cleared conjugate may need to be administered in higher doses than one that is cleared less rapidly. In vivo distribution and localization can be tracked by standard scintigraphic/PET imaging techniques at an appropriate time subsequent to administration, typically between 30 minutes (min) and 180 min and for longer periods such as 3-4 days depending upon the rate of accumulation at the target site with respect to the rate of clearance at the non-target tissue. In vivo distribution and localization can be tracked by standard techniques at a time less 4 days, less than 3 days, less than 2 days, less than 1 day, less than 18 hours (h), less than 12 h, less than 10 h, less than 8 h, less than 6 h, less than 4 h, less than 3.5 h, less than 3 h, less than 2.5 h, less than 2 h, less than 1.5 h, less than 1 h or less than 45 min after administration. In vivo distribution and localization can be tracked by standard techniques at a time greater than 30 min, 45 min, 1 h, 1.5 h, 2 h, 2.5 h, 3 h, 3.5 h, 4 h, 6 h, 8 h, 10 h, 12 h, 18 h, 1 day, 2 days or 3 days after administration. In vivo distribution and localization can be tracked by standard techniques at a time between 30 min and 4 days, 30 min and 3 days, 30 min and 2 days, 30 min and 1 days, 30 min and 18 h, 30 min and 12 h, 30 min and 10 h, 30 min and 8 h, 30 min and 6 h, 30 min and 4 h or 30 min and 3 h after administration.

In a variation, the present invention relates to pharmaceutical compositions. The pharmaceutical composition may contain pharmaceutically acceptable salts, solvates, and prodrugs thereof, and may contain an excipient or other substance necessary to increase the bioavailability or extend the lifetime of the compounds of the present invention.

The pharmaceutical compositions containing a compound of the invention may be in a form suitable for injection either by itself or alternatively, using liposomes, micelles, and/or nanospheres.

A solution of the invention may be provided in a sealed container, especially one made of glass, either in a unit dosage form or in a multiple dosage form.

Any pharmaceutically acceptable salt of a compound, complex or pharmaceutical agent as described herein may be used for preparing a solution of the invention. Examples of suitable salts may be, for instance, the salts with mineral inorganic acids such as hydrochloric, hydrobromic, sulfuric, phosphoric, nitric and the like, and the salts with certain organic acids such as acetic, succinic, tartaric, ascorbic, citric, glutamic, benzoic, methanesulfonic, ethanesulfonic and the like.

Any solvent which is pharmaceutically acceptable and which is able to dissolve the compound, complex or pharmaceutical agent as described herein or a pharmaceutically acceptable salt thereof may be used. The solution of the invention may also contain one or more additional components such as a co-solubilizing agent (which may be the same as a solvent), a tonicity adjustment agent, a stabilizing agent, a preservative, or mixtures thereof. Examples of solvents, co-solubilizing agents, tonicity adjustment agents, stabilizing agents and preservatives which may be suitable for a solution formulation are described below.

Suitable solvents and co-solubilizing agents may include, but are not limited to, water; sterile water for injection (SWFI); physiological saline; alcohols, e.g. ethanol, benzyl alcohol and the like; glycols and polyalcohols, e.g. propyleneglycol, glycerin and the like; esters of polyalcohols, e.g. diacetine, triacetine and the like; polyglycols and polyethers, e.g. polyethyleneglycol 400, propyleneglycol methylethers and the like; dioxolanes, e.g. isopropylidenglycerin and the like; dimethylisosorbide; pyrrolidone derivatives, e.g. 2-pyrrolidone, N-methyl-2-pyrrolidone, polyvinylpyrrolidone (co-solubilizing agent only) and the like; polyoxyethylenated fatty alcohols; esters of polyoxyethylenated fatty acids; polysorbates, e.g., Tween™, polyoxyethylene derivatives of polypropyleneglycols, e.g., Pluronics™.

Suitable tonicity adjustment agents may include, but are not limited to, pharmaceutically acceptable inorganic chlorides, e.g. sodium chloride; dextrose; lactose; mannitol; sorbitol and the like.

Preservatives suitable for physiological administration may be, for instance, esters of parahydroxybenzoic acid (e.g., methyl, ethyl, propyl and butyl esters, or mixtures of them), chlorocresol and the like.

In an embodiment, radioprotectants can also be included in the formulation. These additives include but are not limited to gentisic acid and L-ascorbic acid or combinations thereof.

Suitable stabilizing agents include, but are not limited to, monosaccharides (e.g., galactose, fructose, and fucose), disaccharides (e.g., lactose), polysaccharides (e.g., dextran), cyclic oligosaccharides (e.g., alpha-, beta-, gamma-cyclodextrin), aliphatic polyols (e.g., mannitol, sorbitol, and thioglycerol), cyclic polyols (e.g. inositol) and organic solvents (e.g., ethyl alcohol and glycerol).

The above mentioned solvents and co-solubilizing agents, tonicity adjustment agents, stabilizing agents and preservatives can be used alone or as a mixture of two or more of them in a solution formulation.

In an embodiment, a pharmaceutical solution formulation may comprise the compound, complex or pharmaceutical agent as described herein or a pharmaceutically acceptable salt thereof, and an agent selected from the group consisting of sodium chloride solution (i.e., physiological saline), dextrose, mannitol, or sorbitol, wherein the agent is in an amount of less than or equal to 5%. The pH of such a formulation may also be adjusted to improve the storage stability using a pharmaceutically acceptable acid or base.

In the solutions of the invention the concentration of the compound, complex or pharmaceutical agent as described herein or a pharmaceutically acceptable salt thereof may be less than 100 mg/mL, or less than 50 mg/mL, or less than 10 mg/mL, or less than 5 mg/mL and greater than 0.01 mg/mL, or between 0.5 mg/mL and 5 mg/mL, or between 1 mg/mL and 3 mg/mL. In an embodiment, the concentration that is used is the ideal concentration to be sufficiently cytotoxic to the cancer cells yet limit the toxicity on other cells.

Suitable packaging for the pharmaceutical solution formulations may be all approved containers intended for parenteral use, such as plastic and glass containers, ready-to-use syringes and the like. In an embodiment, the container is a sealed glass container, e.g. a vial or an ampoule. A hermetically sealed glass vial is particularly preferred.

In an embodiment, the packaging may include cGMP/cGLP/cGCP/cGPvP as packaging.

According to an embodiment of the present invention, there is provided, in a sealed glass container, a sterile, injectable solution comprising one or more of the complexes, pharmaceutical agents and compositions as described herein or a pharmaceutically acceptable salt thereof in a physiologically acceptable solvent, and which has a pH of from 2.5 to 3.5. In some embodiments, the complex, pharmaceutical agent or composition of the invention comprises two different radionuclides, preferably wherein one radionuclide is of therapeutic potential and the other radionuclide is of diagnostic potential. Complexes, pharmaceutical agents and compositions comprising one radionuclide are preferred as bound to either the first chelating ligand or the second chelating ligand. Complexes, pharmaceutical agents and compositions comprising ⁸⁹Zr are preferred as bound to the first chelating ligand. Complexes, pharmaceutical agents and compositions comprising a radionuclide other than ⁸⁹Zr are preferred as bound to the second chelating ligand. Complexes, pharmaceutical agents and compositions comprising two different radionuclides may be preferred with ⁸⁹Zr bound to the first chelating ligand and a radionuclide other than ⁸⁹Zr bound to the second chelating ligand. Complexes, pharmaceutical agents and compositions comprising one radionuclide or two different radionuclides and one or more of a protein, peptide, antibody and nanoparticle are preferred. Complexes, pharmaceutical agents and compositions comprising one radionuclide as ⁸⁹Zr bound to the first chelating ligand or a radionuclide other than ⁸⁹Zr bound to the second chelating ligand and one or more of a protein, peptide, antibody and nanoparticle are preferred. Complexes, pharmaceutical agents and compositions comprising two different radionuclides as ⁸⁹Zr bound to the first chelating ligand and a radionuclide other than ⁸⁹Zr bound to the second chelating ligand and one or more of a protein, peptide, antibody and nanoparticle are preferred. For solution formulations, various compounds of the present invention may be more soluble or stable for longer periods in solutions at a pH lower than 6. In one embodiment, the pH of the biomolecule (such as a protein, peptide or antibody) conjugated to the radionuclide should be in the range of 6.5-7 so that it is suitable for injection into an individual (e.g., a human). Further, acid salts of the compounds of the present invention may be more soluble in aqueous solutions than their free base counter parts, but when the acid salts are added to aqueous solutions the pH of the solution may be too low to be suitable for administration. Thus, solution formulations having a pH above pH 4.5 may be combined prior to administration with a diluent solution of pH greater than 7 such that the pH of the combination formulation administered is pH 4.5 or higher. In one embodiment, the diluent solution comprises a pharmaceutically acceptable base such as sodium hydroxide. In another embodiment, the diluent solution is at pH of between 10 and 12. In another embodiment, the pH of the combined formulation administered is greater than 5.0. In another embodiment, the pH of the combined formulation administered is between pH 5.0 and 7.0.

The invention also provides a process for producing a sterile solution with a pH of from 2.5 to 3.5 which process comprises dissolving the compound, complex, pharmaceutical agent or composition as described herein or a pharmaceutically acceptable salt thereof in a pharmaceutically acceptable solvent. Where a pharmaceutically acceptable acid salt of the compound, complex, pharmaceutical agent or composition as described herein is used the pH of the solution may be adjusted using a pharmaceutically acceptable base or basic solution adding a physiologically acceptable acid or buffer to adjust the pH within a desired range. The method may further comprise passing the resulting solution through a sterilizing filter.

In some embodiments, the compound, pharmaceutical agent, complex or composition of the invention may be administered in combination with a further active pharmaceutical ingredient (API). The API may be any that is suitable for treating or diagnosing any of the diseases, conditions and/or disorders that the radionuclide of pharmaceutical potential is suitable for the treatment or diagnosis of, such as a neoplastic disorder. The compound, pharmaceutical agent, complex or composition of the invention may be co-formulated with the further API in any of the pharmaceutical compositions described herein, or the compound, pharmaceutical agent, complex or composition of the invention may be administered in a concurrent, sequential or separate manner. Concurrent administration includes administering the compound, pharmaceutical agent, complex or composition of the invention at the same time as the other API, whether coformulated or in separate dosage forms administered through the same or different route. Sequential administration includes administering, by the same or different route, the compound, pharmaceutical agent, complex or composition of the invention and the other API according to a resolved dosage regimen, such as within about 0.5, 1, 2, 3, 4, 5, or 6 hours of the other. When sequentially administered, the compound, pharmaceutical agent, complex or composition of the invention may be administered before or after administration of the other API. Separate administration includes administering the compound, pharmaceutical agent, complex or composition of the invention and the other API according to regimens that are independent of each other and by any route suitable for either active, which may be the same or different

The methods may comprise administering the compound, pharmaceutical agent, complex or composition of the invention in any pharmaceutically acceptable form. The pharmaceutical composition may comprise any pharmaceutically acceptable excipient described herein.

The compounds, pharmaceutical agents, complexes or compositions of the invention may be administered by any suitable means, for example, parenterally, such as by subcutaneous, intraperitoneal, intravenous, intramuscular, or intracisternal injection, infusion or implantation techniques (e.g., as sterile injectable aqueous or non-aqueous solutions or suspensions).

The compounds, pharmaceutical agents or complexes of the invention may be provided as any of the pharmaceutical compositions described herein.

### Methods of Treatment

Any of the compounds, complexes, compositions and agents described herein may be used to treat any disease or condition treatable with a nuclide of pharmaceutical potential complexed with the compounds prior to administration.

Radionuclides are typically used in treating neoplastic disorders including cancer, and treatment with a radionuclide may be considered as internal radiotherapy.

Accordingly, there is provided a method of treating a neoplastic disorder, comprising administering to a subject in need thereof a therapeutically effective amount of a complex of the invention. The complex may be administered in the form of a pharmaceutical agent or composition. Any suitable agent or composition described herein may be used.

The complex of the invention may be any of the complexes described herein where the nuclide is of therapeutic potential. The complex may be in the form of a pharmaceutical agent or composition of the invention. The complex may further comprise a nuclide of diagnostic potential such that the method of treatment is also a method of diagnosis.

The method may further include the step contacting a compound or composition of the invention with a metal of therapeutic potential to form a complex of the invention.

In a further aspect, there is provided use of a complex of the present invention in the manufacture of a medicament for the treatment of a neoplastic disorder.

In a further aspect, there is provided use of a compound of the invention in the manufacture of a medicament for the treatment of a neoplastic disorder, wherein the medicament comprises the compound complexed with a nuclide of pharmaceutical potential.

In a further aspect, there is provided use of a nuclide of pharmaceutical potential in the manufacture of a medicament for the treatment of a neoplastic disorder, wherein the medicament comprises the compound complexed with a nuclide of pharmaceutical potential.

In a further aspect, there is provided use of a complex of the present invention in the treatment of a neoplastic disorder.

In a further aspect, there is provided a complex of the present invention for use in the treatment of a neoplastic disorder.

In some embodiments of the present invention, the complex incorporates a targeting moiety that directs the compound to a targeted tissue, organ, receptor or other biologically expressed composition to enable targeted delivery of the nuclide to a cancer. In some embodiments of the present invention, the complex incorporates the targeting moiety girentuximab.

In some embodiments, the nuclide of pharmaceutical potential is selected from the group consisting of ⁹⁰Y, ¹⁵³Sm, ¹⁶¹Tb, ¹⁷⁷Lu, ²¹³Bi and ²²Ac; preferably ¹⁷⁷Lu and ²²⁵Ac; more preferably ¹⁷⁷Lu.

In some embodiments wherein the complex further comprises a nuclide of diagnostic potential it is preferred that the nuclide of diagnostic potential is ⁸⁹Zr and the nuclide of therapeutic potential is ²²⁵Ac or ¹⁷⁷Lu, preferably ¹⁷⁷Lu.

Neoplastic disorders include malignant and benign cancerous growths. In some embodiments, the treatment is for cancer. In some embodiments, the treatment is for a cancer with a cognate antigen. In some embodiments, the treatment is for cancer selected from the group consisting of prostate cancer including castrate-resistant metastatic prostate cancer, breast cancer, renal cancer including metastatic clear cell renal cell cancer, pancreatic cancer, lung cancer, gastric cancer or metastatic bone disease. In some embodiments, the treatment is for prostate cancer, such as castrate-resistance metastatic prostate cancer. In some embodiments, the treatment is for breast cancer. In some embodiments, the treatment is for pancreatic cancer. In some embodiments, the cancer with a cognate antigen is PSMA and the cancer is selected from the group consisting of: a prostate tumor or cell, a metastasized prostate tumor or cell, a lung tumor or cell, a renal tumor or cell, a glioblastoma, a pancreatic tumor or cell, a bladder tumor or cell, a sarcoma, a melanoma, a breast tumor or cell, a colon tumor or cell, a germ cell, a pheochromocytoma, an esophageal tumor or cell, a stomach tumor or cell, and combinations thereof. In some embodiments, the cancer with a cognate antigen is carbonic anhydrase IX and the cancer is metastatic clear cell renal cell cancer.

In some embodiments, the cancer is in vitro, in vivo, or ex vivo. In particular embodiments, the cancer is present in a subject.

A “cancer” in an animal refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within an animal, or circulate in the blood stream as independent cells.

As used herein, the term “effective amount” means that amount of a drug or pharmaceutical agent that will elicit the biological, physical or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. Furthermore, the term “diagnostically effective amount” means any amount which, as compared to a corresponding subject who has not received such amount, results in improved diagnosis, imaging, or assessment of the health or likely capability of a subject, organ or tissue.

### Methods of Diagnosis

In a further aspect, there is provided a method of diagnosis in a subject in need thereof, the method comprising administering to the subject a diagnostically effective amount of a complex comprising a compound of the invention and a nuclide of diagnostic potential.

The complex may be any of complex of the compounds of the invention and nuclides of diagnostic potential described herein. The complex may further comprise a nuclide of therapeutic potential such that the method of diagnosis is also a method of therapy.

The method may further include the step of contacting a compound or composition of the invention with a metal of diagnostic potential to form a complex of the invention.

In a further aspect, there is provided use of a complex of the present invention in the manufacture of a medicament for the diagnosis of a neoplastic disorder.

In a further aspect, there is provided use of a compound of the present invention in the manufacture of a medicament for the diagnosis of a neoplastic disorder, wherein the medicament comprises a complex of the compound and a nuclide of diagnostic potential.

In a further aspect, there is provided use of a nuclide of diagnostic potential in the manufacture of a medicament for the diagnosis of a neoplastic disorder, wherein the medicament comprises a complex of the compound and a nuclide of diagnostic potential.

In a further aspect, there is provided a complex of the present invention for use in the diagnosis of a neoplastic disorder.

In some embodiments of the present invention, the complex incorporates a targeting moiety that directs the compound to a targeted tissue, organ, receptor or other biologically expressed composition to enable targeted delivery of the nuclide to a cancer. In some embodiments of the present invention, the complex incorporates the targeting moiety girentuximab.

In some embodiments, the nuclide of diagnostic potential is a nuclide suitable for PET imaging, preferably selected from ⁸⁹Zr and ⁹⁰Nb; most preferably ⁸⁹Zr.

In some embodiments, the nuclide of diagnostic potential is a nuclide suitable for MRI, preferably Gd.

In some embodiments wherein the complex further comprises a nuclide of therapeutic potential it is preferred that the nuclide of diagnostic potential is ⁸⁹Zr and the nuclide of therapeutic potential is ²⁵⁵Ac or ¹⁷⁷Lu, preferably ¹⁷⁷Lu.

In some embodiments, the diagnostic method comprises subjecting the subject to positron emission tomography (PET) imaging, preferably immuno-PET imaging. PET imaging is a functional imaging technique applied in nuclear medicine, whereby a three-dimensional image (e.g. of functional processes) in the body is produced. The system detects pairs of gamma rays emitted indirectly by a positron-emitting radionuclide, which is introduced into the body in form of a pharmaceutical compound.

In some embodiments, the diagnostic method comprises subjecting the subject to magnetic resonance imaging (MRI), preferably wherein the nuclide of diagnostic potential is Gd.

In some embodiments, the diagnosis is for a neoplastic disorder. In some embodiments, the diagnosis is for cancer. The methods of diagnosis may be applied to any of the cancers described herein for therapy. It will be appreciated that the compounds and compositions of the invention may be applied for therapy or diagnosis through selection of the complexing metal selected

In some embodiments, the diagnostic method of the invention may be used in combination with another diagnostic method, such as magnetic resonance imaging (MRI), radiography, ultrasound, elastography, photoacoustic imaging, tomography (including computed tomography) and echocardiography; preferably magnetic resonance imaging (MRI) and tomography (including computed tomography).

### Methods of Production

In a further aspect, there is provided a method of producing a compound or composition of the present invention.

Typically, the compounds or compositions of the invention may be prepared by techniques known in the art. The specific reagents and conditions for effecting each of these steps will depend on the specific substituents selected for each reaction partner. The skilled person would readily appreciate how to determine and/or optimise these reagents and conditions. Similarly, where a starting material is not commercially available, the skilled person would be able to design and implement its preparation based on techniques and reactions previously described. Embodiments of these steps are provided in the Examples with reference to specific compounds described herein.

Reagents for preparation of the compounds of the present invention can be obtained from any source. A wide range of sources are known to those of ordinary skill in the art. The reagents can be synthetic, or obtained from natural sources. Reagents can be of any purity, for example, reagents may be isolated and purified using any technique known to those of ordinary skill in the art.

Any method known to those of ordinary skill in the art can be used to conjugate a chelating ligand moiety, linker moiety, a targeting moiety, a substituent capable of conjugation to a targeting moiety, or a substituent moiety, to the appropriate moiety of the compound. Reactions can be carried out in an aqueous medium or a non-aqueous medium. Any ratio of reagents can be used in a reaction mixture. The product of a reaction can be used immediately, stored or further processed to enhance stability through processes such as freeze-drying before storage.

In some embodiments, a bond between one or more of the chelating ligands and the linker group, or between the two different chelating ligands if the linker is a bond, is an amide bond. Any method known in the art can be used to form amide bonds. Preferred amide bond forming reagents include those that proceed via mixed carboxylic and carbonic anhydrides (such as PivCl, Boc₂O and EEDQ), those that proceed via sulfonate-based anhydrides (such as TsCl), those that proceed via phosphorus-based anhydrides (such as T3P), those that proceed via activated esters (such as NHS) carbodiimides (such as DCC and EDC), guanidinium and uranium salts (such as HBTU and TPTU), triazine-based reagents (such as cyanuric chloride) and boron species (such as boric acid). Amide bond forming reagents that proceed via activated esters (such as NHS), carbodiimides (such as DCC and EDC) and guanidinium and uranium salts (such as HBTU and TPTU) are especially preferred. NHS, EDC and HBTU are particularly preferred amide bond forming reagents.

In an embodiment, a compound of the invention is formed by the reaction between A-COOH and H₂NL¹-COOH under amide bond forming conditions, followed by reaction of the resulting product with H₂N—B under amide bond forming reaction conditions,

wherein L¹ is the portion of the linker L that does not comprise the amine and carboxylic acid groups depicted in L¹.

In an embodiment, a compound of the invention is formed by the reaction between H₂N—B and H₂NL¹-COOH under amide bond forming conditions, followed by reaction of the resulting product with A-COOH under amide bond forming reaction conditions,

wherein L¹ is the portion of the linker L that does not comprise the amine and carboxylic acid groups depicted in L¹.

In an embodiment, a compound of the invention is formed by the reaction between HOOC—B and H₂NL¹-COOH under amide bond forming conditions, followed by reaction of the resulting product with A-NH₂ under amide bond forming reaction conditions,

wherein L¹ is the portion of the linker L that does not comprise the amine and carboxylic acid groups depicted in L¹.

In an embodiment, a compound of the invention is formed by the reaction between A-NH and H₂NL¹-COOH under amide bond forming conditions, followed by reaction of the resulting product with HOOC—B under amide bond forming reaction conditions,

wherein L¹ is the portion of the linker L that does not comprise the amine and carboxylic acid groups depicted in L¹.

In an embodiment, a compound of the invention is formed by the reaction between A-NH and HOOC—B.

In an embodiment, a compound of the invention is formed by the reaction between A-COOH and HN—B.

Any method known to those of ordinary skill in the art can be used to purify a compound or composition of the invention. In some embodiments, the compound or composition is purified by solvent extraction or trituration. In some embodiments, the compound or composition is purified via liquid chromatography, high-performance liquid chromatography (HPLC), size exclusion chromatography (gel permeation chromatography), affinity chromatography, or ion exchange chromatography. In some embodiments, the compound or composition is purified by high-performance liquid chromatography (HPLC). In some embodiments, the compound or composition is isolated by high-performance liquid chromatography (HPLC).

The compounds, compositions, kits and methods described herein are described by the following illustrative and non-limiting examples.

It will be understood that the invention disclosed and defined in this specification extends to all alternative combinations of two or more of the individual features mentioned or evident from the text or drawings. All of these different combinations constitute various alternative aspects of the invention.

The methods and compounds described herein are described by the following illustrative and non-limiting examples.

### EXAMPLES

### General Comments Regarding the Synthesis

The synthesis of the two-component system (DFOB-DOTA (2)), the three-component system (DFOB-L-LYS-DOTA (3)), and the four-component system (DFOB-PPH-L-LYS-DOTA (4)) requires the formation of one, two or three amide bonds, respectively. Amide bond forming chemistry can be undertaken using different approaches. In one approach, the carboxylic acid containing motif can be activated using N-hydroxysuccinimide (NHS) and reacted with an amine-bearing fragment in the presence of base. This chemistry requires the NHS-activated component be first isolated before the next reaction. In another pathway, carboxylic acid groups can be activated in situ using reagents such as N,N,N′,N′-tetramethyl-O-(1H-benzotriazol-1-yl)uronium hexafluorophosphate (HBTU) with the amine-bearing fragment in the presence of base subsequently introduced directly into this mixture. This can be advantageous, since the reaction can be conducted in one step (that is, a ‘one-pot’ reaction). The NHS-route was used to prepare the two-component system (DFOB-DOTA (2)). The HBTU-route was used to prepare the three-component system (DFOB-L-LYS-DOTA (3)), and the four-component system (DFOB-PPH-L-LYS-DOTA (4)).

### Instrumentation

Mass spectra were obtained using a reverse-phase liquid chromatography-mass spectrometry instrument with an autoinjector (100 μL loop), an Agilent 1260 Infinity degasser, a quaternary pump and an Agilent 6120 Series Quadrupole electrospray ionization (ESI)-mass spectrometer. An Agilent C18 column reverse-phased prepacked column (4.6×150 mm i.d., 0.5 mL min⁻¹, particle size 5 μm) was used for all experiments. The following instrument conditions were used: 5 μL injection volume, 4 kV spray voltage, 3 kV capillary voltage, 250° C. capillary temperature, and a 10 V tube lens-offset. The mobile phase was prepared by mixing acetonitrile:formic acid (99.9:0.1) (ACN:FA) and H₂O:formic acid (99.9:0.1). The method used a 5-95% ACN:H₂O gradient with a flow rate of 0.5 mL min⁻¹ over 40 min or a flow rate of 0.8 mL min⁻¹ over 25 min, as required. Spectral data were acquired and processed using Agilent OpenLAB Chromatography Data System ChemStation Edition. Preparative high-performance liquid chromatography (HPLC) was conducted on a Shimadzu LC-20 series LC system with two LC-20AP pumps, an SIL-10AP autosampler, a SPD-20A UV/VIS detector, and an FRC-10A fraction collector. A Shimadzu Shimpack GIS column (150×20 mm i.d., particle size 5 μm) was used for semipreparative purification at a flow rate of 20 mL min⁻¹. The organic phase (B) consisted of ACN:TFA (99.95:0.05). The aqueous phase (A) consisted of H₂O:TFA (99.95:0.05). Spectral data were acquired and processed using Shimadzu LabSolutions Software (version 5.73).

### Materials

DOTA tri(tert-butyl) ester (1b) (97%) was obtained from Arctom Chemicals. N-(3-Dimethylaminopropyl)-N′-ethylcarbodiimide hydrochloride (99%) was obtained from Chem-Impex. Acetonitrile-190, toluene (≥99.5%), ammonia solution (28%) and diethyl ether were obtained from Ajax Finechem. N-Hydroxysuccinimide (98%), desferrioxamine B mesylate (≥92.5%) (1a), triethylamine (≥99%), trifluoroacetic acid (99%), triisopropylsilane (98%), N,N,N′,N′-tetramethyl-O-(1H-benzotriazol-1-yl)uronium hexafluorophosphate (≥98%) (HBTU), 1,4-phenylenediisothiocyanate (98%), sodium bicarbonate (≥99.7%), sodium sulfate (anhydrous, ≥99%), N,N-dimethylformamide (99.8%), piperidine (99%), sodium hydroxide (anhydrous, ≥98%), hydrochloric acid (37%), lutetium chloride (99.9%), zirconium chloride (≥99.9%) and zirconium acetylacetonate (98%) were obtained from Sigma-Aldrich. Milli-Q water was prepared using a Millipore 0-pod system. Fmoc-L-LYS-DOTA(O^{t}Bu)₃ (1c) was obtained from Macrocyclics. N,N-diisopropylethylamine was obtained from Sigma-Aldrich (99.5%) and Merck (98%). Dichloromethane was obtained from Ajax Finechem and Merck. Methanol was obtained from Ajax Finechem and Chem-supply (≥99.9%). Ammonium acetate (≥97%) was obtained from APS Finechem. Girentuximab was obtained from Telix Pharmaceuticals Pty Ltd.

### Example 1

### Synthesis of DFOB-DOTA (2)

The synthesis of DFOB-DOTA (2), is detailed below.

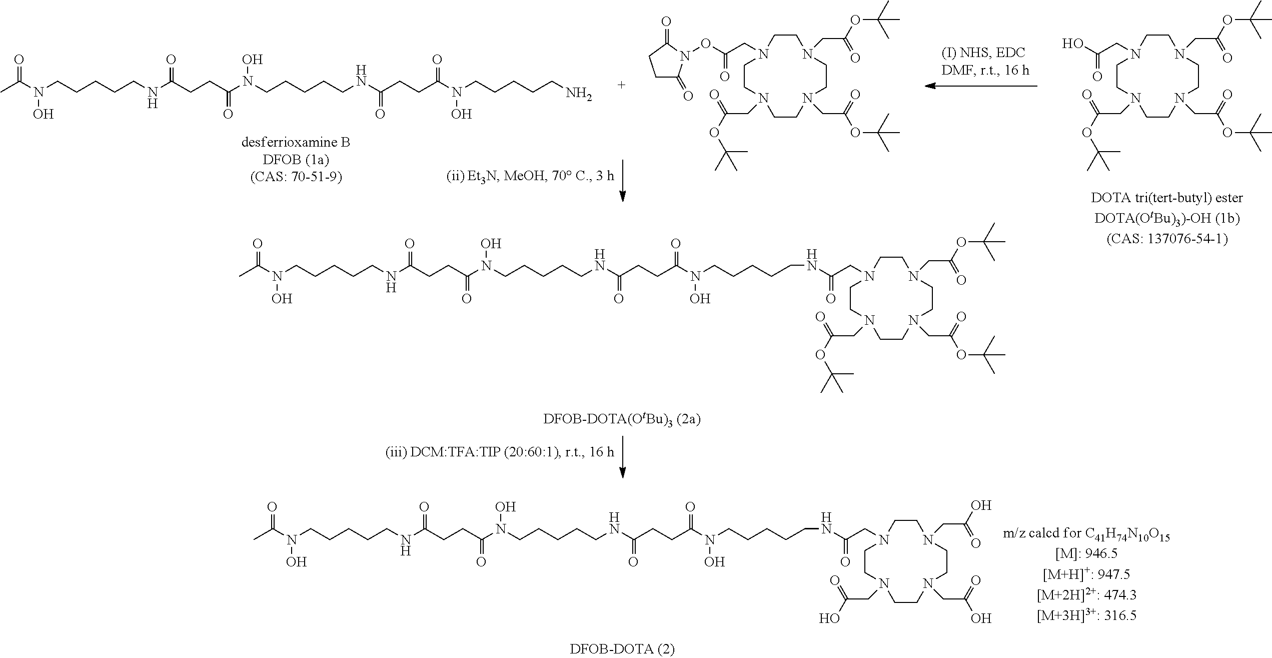

DOTA tri(tert-butyl) ester (2-(4,7,10-Tris(2-(tert-butoxy)-2-oxoethyl)-1,4,7,10-tetraazacyclododecan-1-yl)acetic acid) (359.6 mg, 0.63 mmol) (1b) was dissolved in DCM (35.8 mL) to which N-hydroxysuccinimide (NHS) (108.3 mg, 0.94 mmol) was added, immediately followed by N-(3-dimethylaminopropyl)-N′-ethylcarbodiimide hydrochloride (EDC) (488.7 mg, 3.15 mmol). The mixture was left to stir at ambient temperature for 16 h. Water (35 mL) was added to the reaction solution and after agitation the organic layer was collected. The aqueous layer was further extracted with DCM (3×35 mL) and the organic layers were combined. The solvent was removed in vacuo (external bath 45° C.) to yield NHS-activated DOTA tri(tert-butyl) ester (tri-tert-butyl 2,2′,2″-(10-(2-((2,5-dioxopyrrolidin-1-yl)oxy)-2-oxoethyl)-1,4,7,10-tetraazacyclododecane-1,4,7-triyl)triacetate) as a yellow oil. ESI-MS positive ion calculated for C₃₂H₅₅N₅O₁₀: [M+H]⁺ 670.39.

A solution of desferrioxamine B mesylate (134.4 mg, 0.20 mmol) (1a) in MeOH (5 mL) was added to a solution of NHS-activated DOTA tri(tert-butyl) ester (212.8 mg, 0.32 mmol) and triethylamine (67 μL, 0.48 mmol) in MeOH (5 mL). The resultant solution was stirred under reflux at 70° C. for 3 h. The solvent was removed in vacuo and the residue was washed with cold diethyl ether (3×10 mL) and then water (20 mL) was added and the slurry transferred to a 50-mL Falcon tube. The solid was separated via centrifugation, transferred to a round bottom flask before removing any remaining solvent in vacuo (external bath 45° C.), which yielded DFOB-DOTA tri(tert-butyl) ester as a white solid. ESI-MS positive ion calculated for C₅₃H₉₈N₁₀O₁₅: [M+H]⁺ 1115.72.

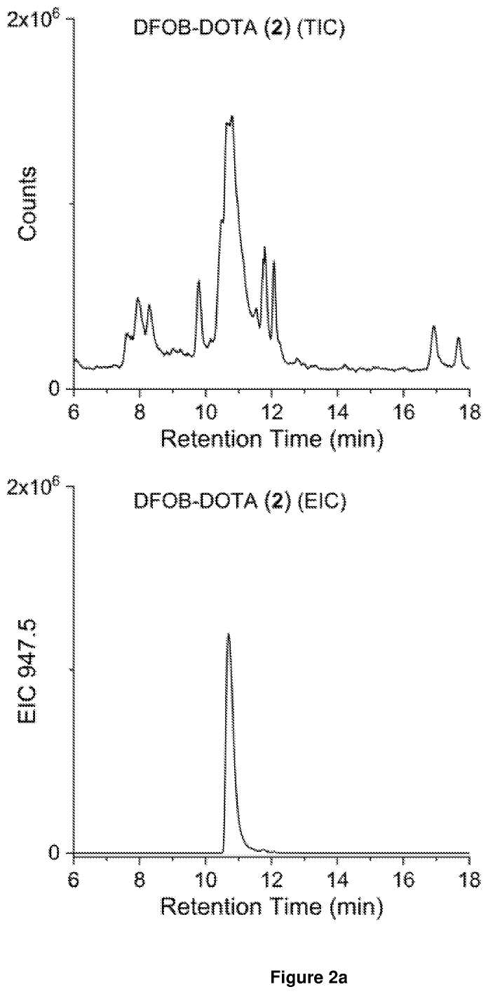

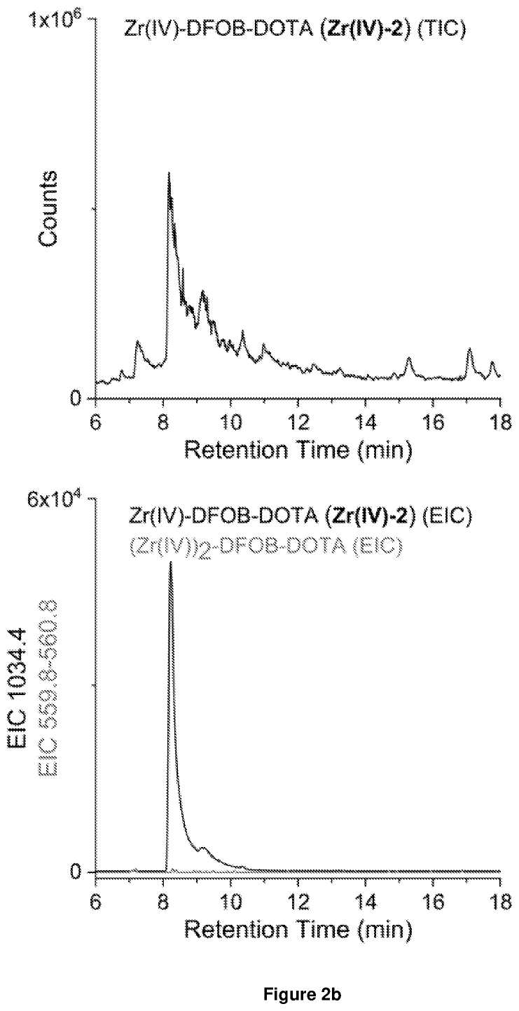

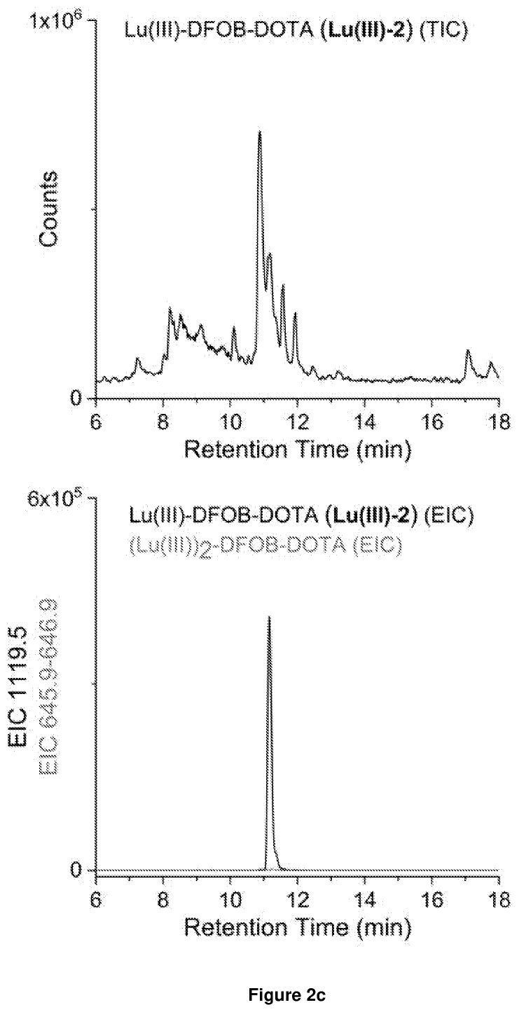

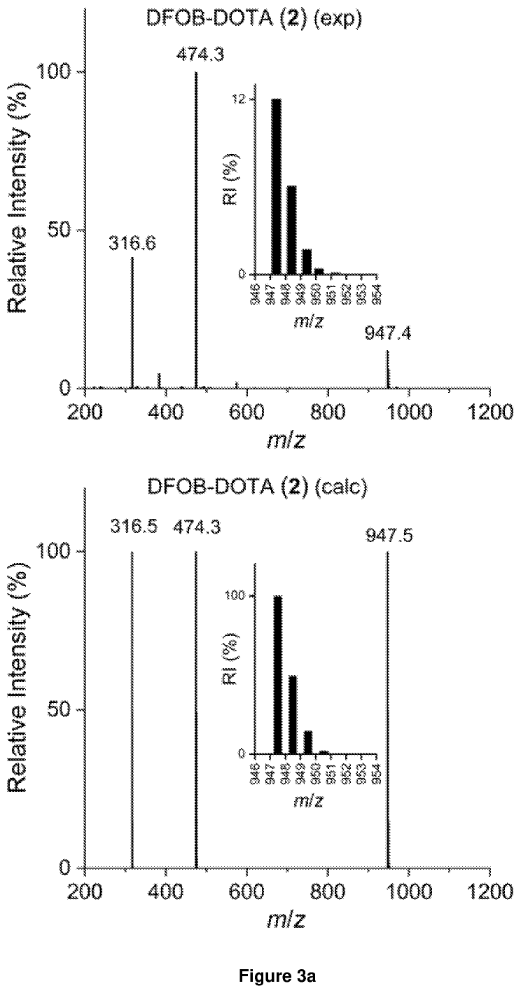

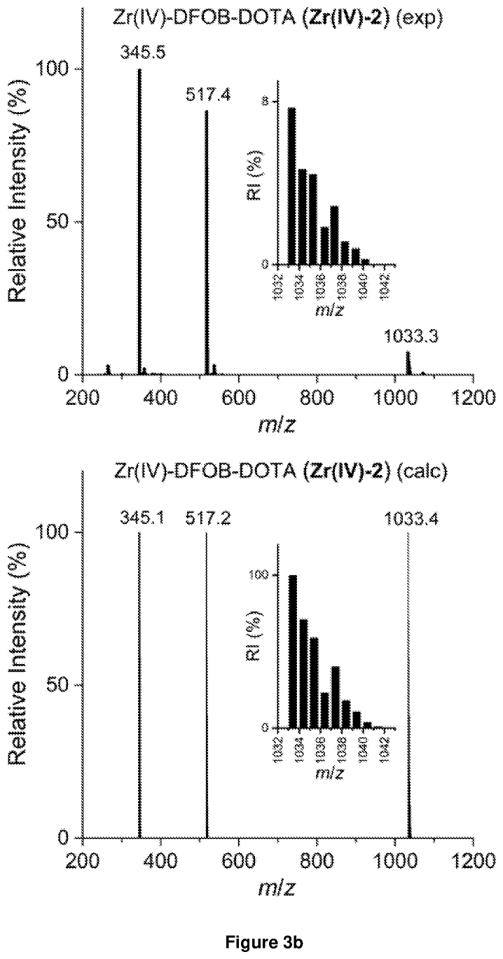

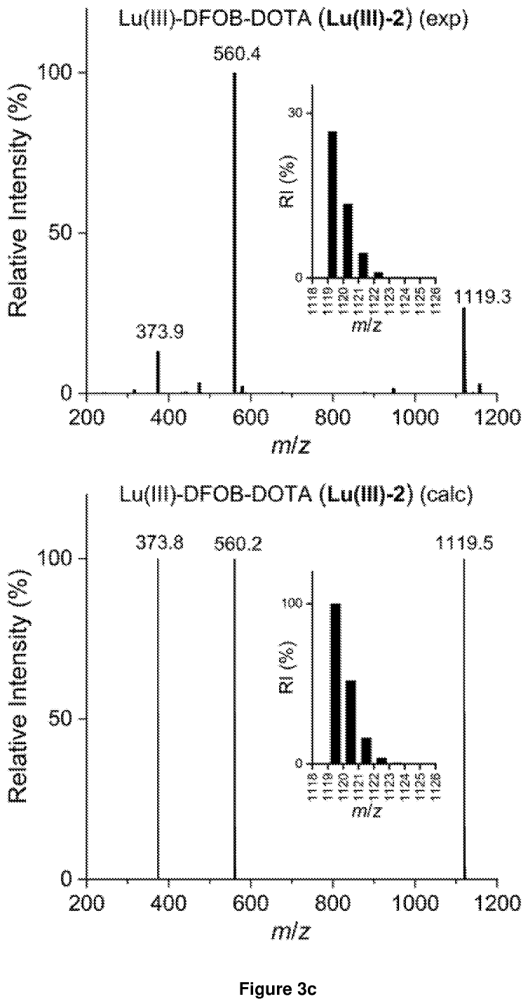

DFOB-DOTA tri(tert-butyl) ester (179.7 mg, 0.16 mmol) was dissolved in a mixture of DCM:TFA:TIPS, 1.47 mL:5.13 mL:87 μL, and the solution was stirred at room temperature for 16 h. The solvent was removed in vacuo (external bath 45° C.). Cold diethyl ether (10 mL) was added to the residue, which extracted the product into the solvent phase. The slurry was transferred to a round bottomed flask before removing the remaining solvent in vacuo (external bath 45° C.) to yield the crude product as a white solid. This product was purified by HPLC using a stepwise gradient of mobile phase B in mobile phase A as follows: 0-12% from 0-7.5 min, 12-22% B from 7.5-17.5 min and 22-40% B from 17.5-20 min with a flow rate of 20 mL min⁻¹. The product was collected at 14.43 min, with these fractions pooled and the solvent removed by lyophilisation, to yield DOTA-DFOB as a white solid (4.08 mg, 2.16%). Note: An accurate yield was unable to be calculated. During lyophilisation, the freeze-dryer malfunctioned, which lead to the loss of a significant amount of material. Sufficient material was obtained for analytical measurements. ESI-MS positive ion calculated for C₄₁H₇₄N₁₀O₁₅: [M+H]+ 947.53. Refer to FIG. 2a and FIG. 3a.

### Example 2

### Selective Complex Formation Upon Exposure of DFOB-DOTA (2) to Different Metal Ions

The selective metal complexation of single chelating ligands of an embodiment of DFOB-DOTA (2), is detailed below.

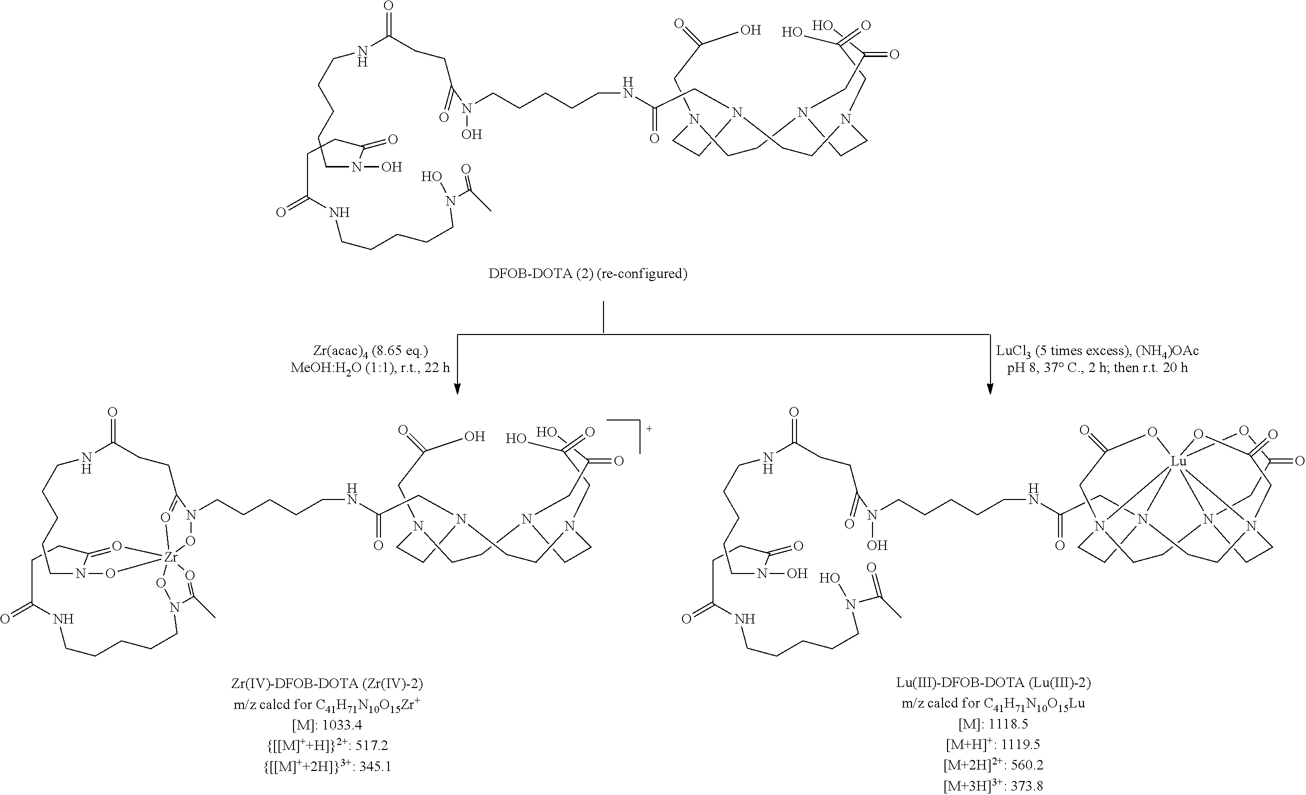

DFOB-DOTA (2) was mixed with separately mixed with solutions comprising Zr(IV) and Lu(III). In both cases, ESI-MS analysis was consistent with only one of the two chelating ligands of DFOB-DOTA (2) forming a complex with the metal ion. The known chelating ligands affinities for Zr(IV) and Lu(III) are consistent with the two metals forming a complex with different chelating ligands of DFOB-DOTA (2). The inventors expect that ¹³C nuclear magnetic resonance (NMR) spectroscopy experiments would further confirm the chelating ligand selectivity of the metal-compound complexes. FIG. 2 and FIG. 3 provide LCMS and mass spectrometry results for Example 2a and Example 2b.

### Example 2a: Formation of Zr(IV)-DFOB-DOTA (Zr-2)

To a solution of DFOB-DOTA in methanol-water (1:1) was added a solution of Zr(acac)₄ in methanol water (1:1) (8.65 eq.). The mixture was left to stir at ambient temperature for 22 h and the solution was analysed using LCMS. ESI-MS positive ion calculated for C₄₁H₇₁N₁₀O₁₅Zr: [M]⁺ 1033.4. Refer to FIG. 2b and FIG. 3b.

### Example 2b: Formation of Lu(III)-DFOB-DOTA (Lu-2)

To 249 μL of a solution of DOTA-DFOB (2.5 mM) in ammonium acetate solution (0.2 M, pH 8) was added 62 μL of a solution of LuCl₃ (50 mM) in water. The resultant 5:1 solution of Lu(III):DFOB-DOTA (2) was stirred at 37° C. for 2 h and then at ambient temperature for 20 h. The solution was analysed using LCMS. ESI-MS positive ion calculated for C₄₁H₇₁N₁₀O₁₅Lu: [M+H]⁺ 1119.5. Refer to FIG. 2c and FIG. 3c.

### Example 3

### Synthesis of DFOB-L-LYS-DOTA (3)

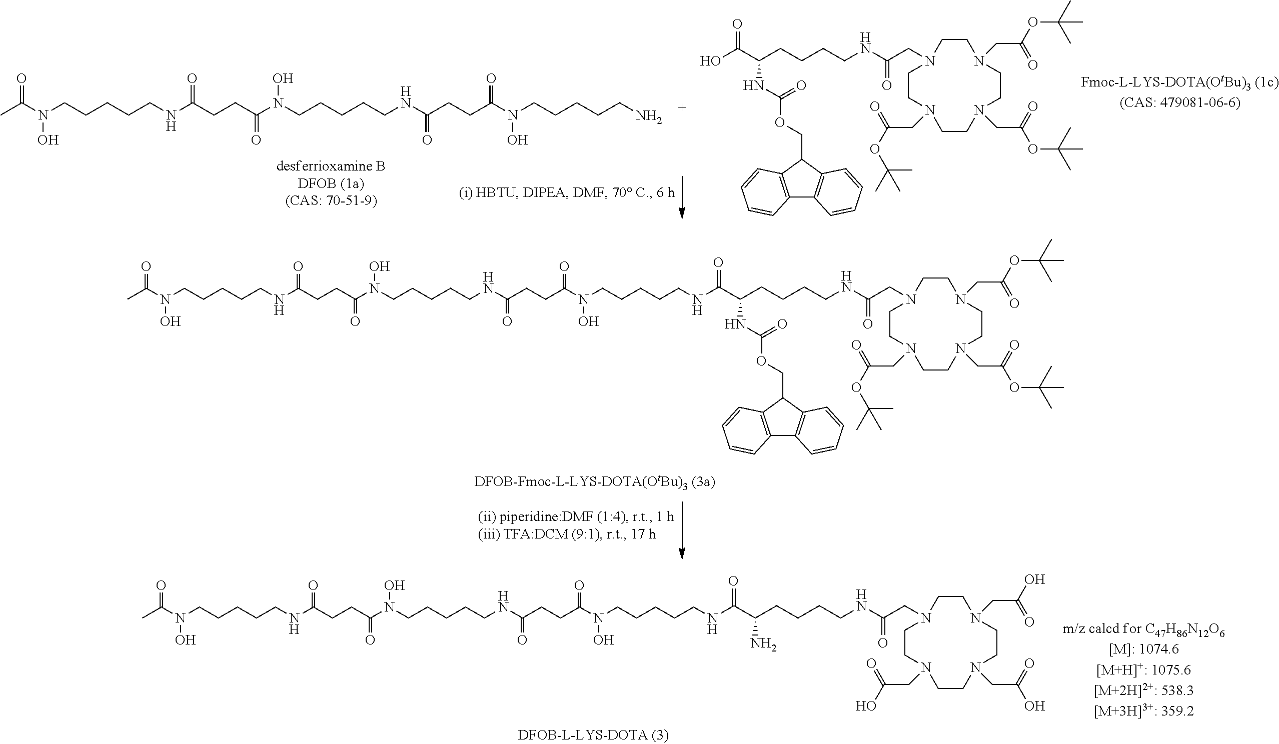

### DFOB-Fmoc-L-LYS-DOTA(OrBu)₃ (3a)

A sample of Fmoc-L-Lys-mono-amide-DOTA-tris(t-Bu ester) (92.1 mg, 86.1 μmol) was dissolved in N,N-dimethylformamide (DMF) (10 mL) followed by the addition of N,N-diisopropylethylamine (DIPEA) (35 μL, 200.9 μmol). The solution was stirred at room temperature (r.t.) for 10 min. N,N,N′,N′-Tetramethyl-O-(1H-benzotriazol-1-yl)uronium hexafluorophosphate (HBTU) (45.3 mg, 119.4 μmol) was added to the solution, which was stirred at r.t. for 30 min.

Desferrioxamine mesylate salt (78.3 mg, 119.2 μmol) was added to the solution, which was heated to 50° C. and stirred for 1 h. A volume of dichloromethane (DCM) (100 mL) was added to the reaction mixture and the mixture was extracted 3 times with 50 mL aliquots of saturated sodium bicarbonate and once with a 50 mL aliquot of brine. The organic layer containing the product was dried with anhydrous sodium sulfate and after vacuum filtration, the solvent was removed in vacuo to yield the semi-pure product tris(tBu)DOTA-(Fmoc)Lys-DFOB as a yellow oil. Crude yield=102.7%. Comment: The Fmoc-L-LYS-DOTA(O^{t}Bu)₃)—OH (1c) scaffold was prepared in-house, based on methods known in the art (for instance, see De León-Rodriguez et al (2004) Solid-phase synthesis of DOTA-peptides, Chem.-Eur. J. 10, 1149-115).

### DFOB-L-LYS-DOTA (3)

A sample of DFOB-Fmoc-L-Lys-mono-amide-DOTA-tris(t-Bu ester) (134.6 mg, 91.9 μmol) was suspended in a solution of piperidine:DMF (1:4, 0.4 mL:1.6 mL), which was stirred at r.t. for 1 h. The solvent was removed in vacuo and the residue was dissolved in TFA:DCM (9:1, 900 μL:100 μL), with stirring at r.t. for 16.5 h. The solvent was removed in vacuo and residual TFA removed by successive treatment (dissolution/in vacuo removal) with methanol and then toluene. The oily residue was suspended in a minimal aliquot of water and the pH of the solution was adjusted to 7 with aliquots of 1 M NaOH or HCl. The solvent was removed in vacuo to give an oily residue, which was dissolved in H₂O:acetonitrile (ACN) 7:3 for HPLC purification. The fraction containing DOTA-Lys-DFOB was collected and the solvent was removed using a high vacuum freeze-drier to give a white powder. Final yield (after HPLC purification): 40.5%.

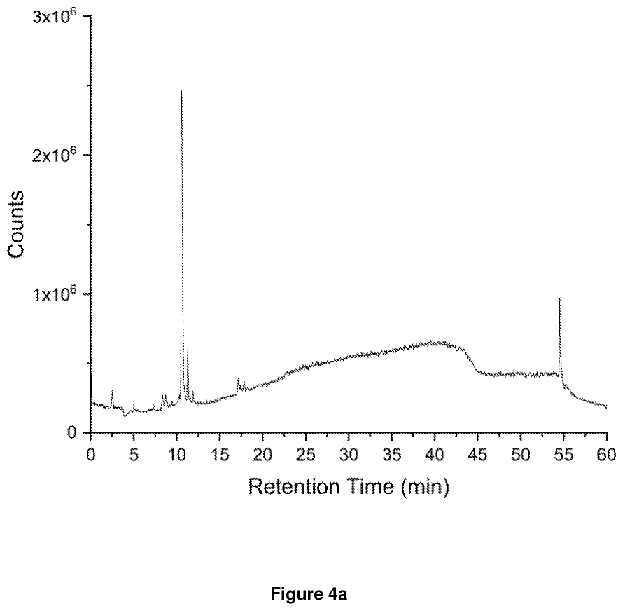

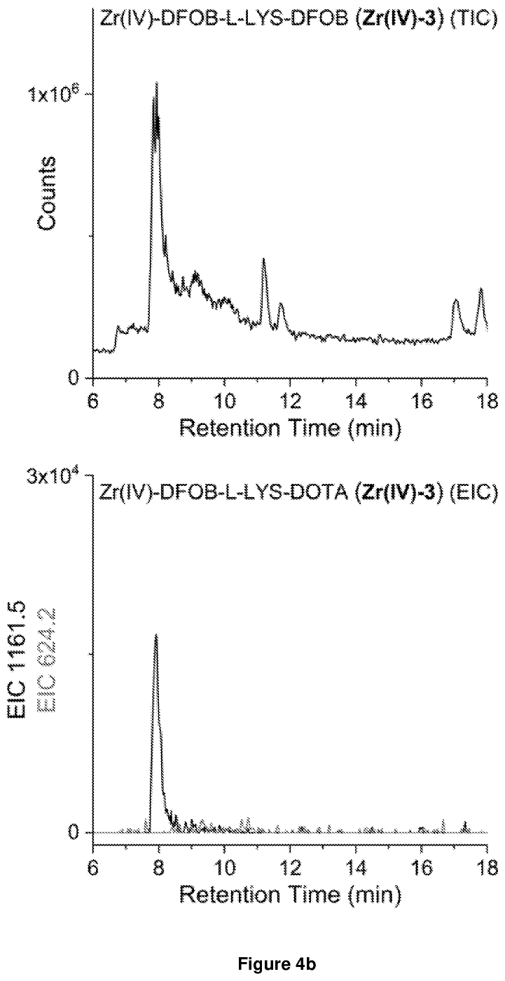

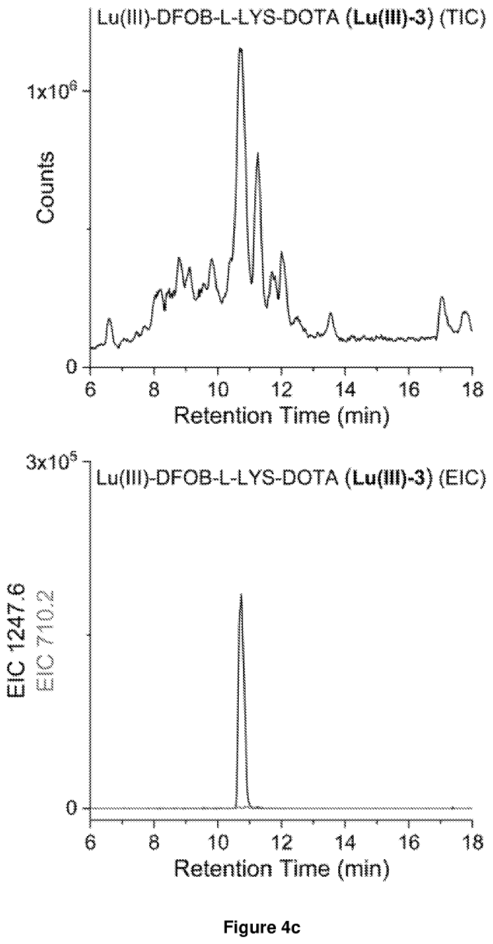

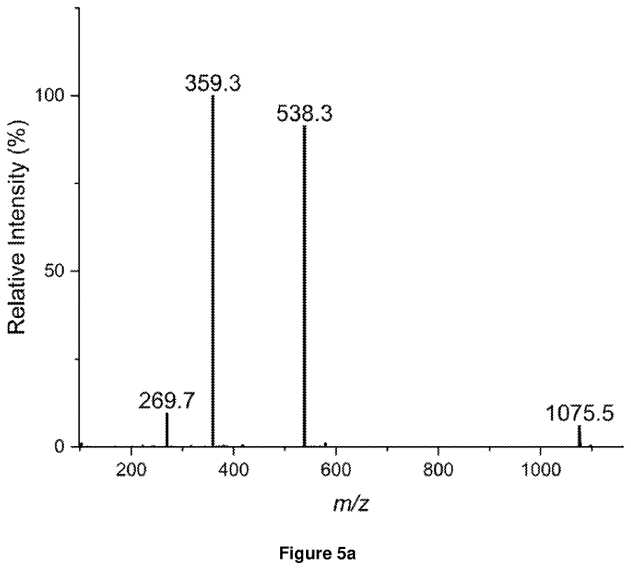

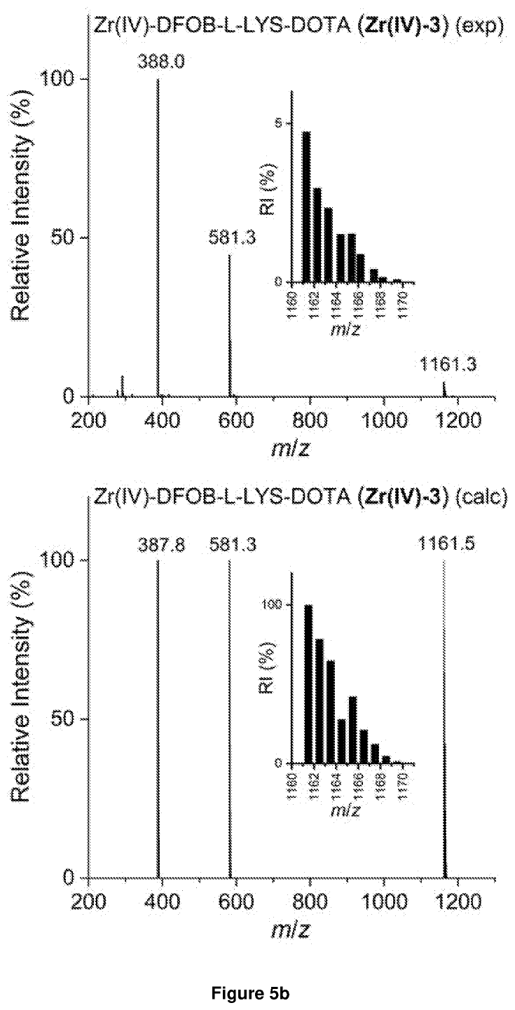

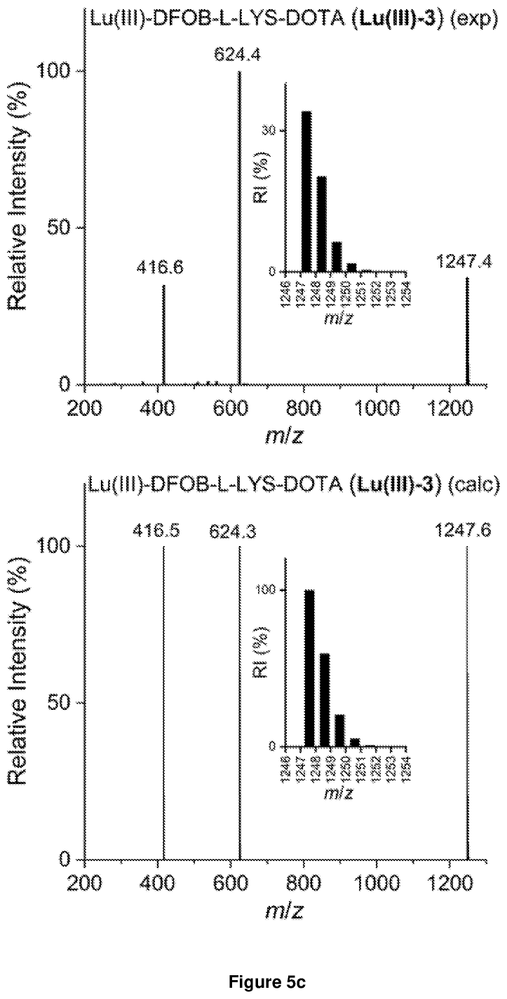

m/z calcd for C₄₇H₈₆N₁₂O₁₆ [M]: [M+H]⁺ 1075.6, [M+2H]²⁺ 538.3, [M+3H]³⁺ 359.2; found 1075.5, 538.4, 359.3. Refer to FIG. 4a and FIG. 5a.

### Example 4

### Selective Complex Formation Upon Exposure of the Three-Component System DFOB-L-LYS-DOTA (3) to Different Metal Ions

The selective metal complexation of single chelating ligands of DFOB-L-LYS-DOTA (3), is detailed below.

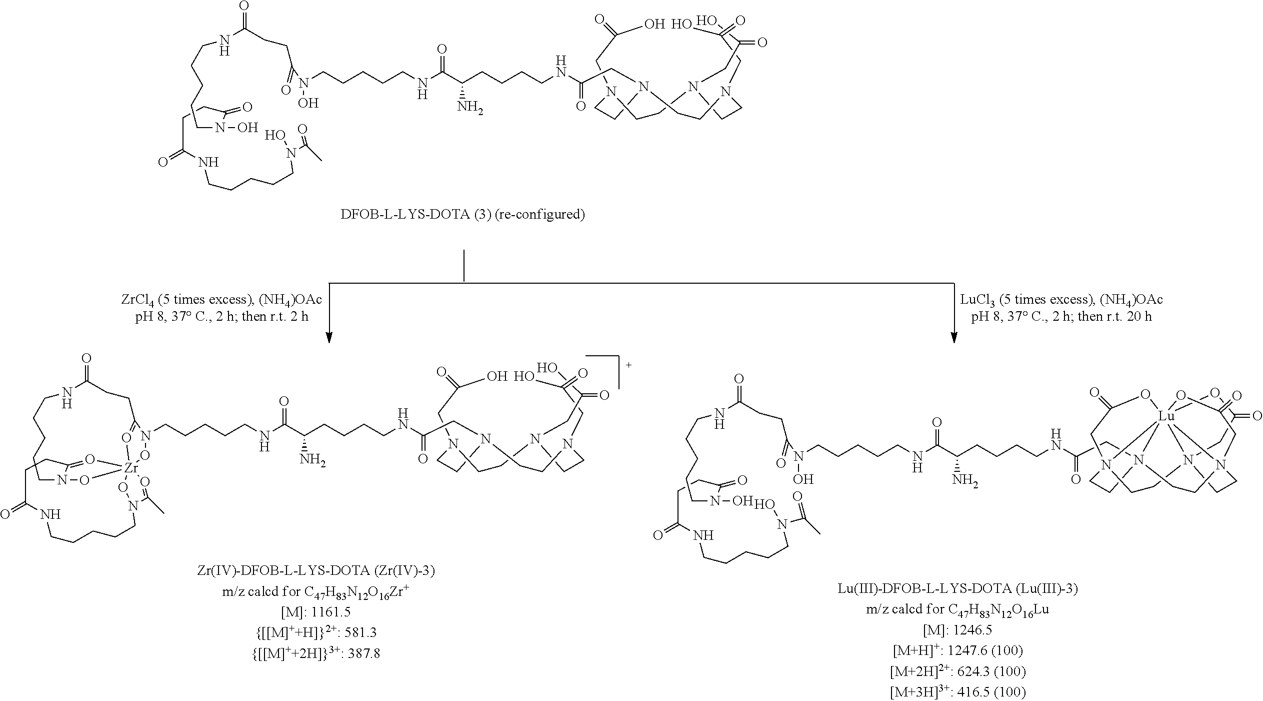

As detailed below, DFOB-L-LYS-DOTA (3) was mixed, in separate experiments, with solutions comprising Zr(IV) and Lu(III). In both cases, ESI-MS analysis was consistent with only one of the two chelating ligands of DFOB-L-LYS-DOTA (3) forming a complex with the metal ion. The known chelating ligands affinities for Zr(IV) and Lu(III) are consistent with the two metals forming a complex with different chelating ligands of DFOB-L-LYS-DOTA (3). FIG. 4 and FIG. 5 provide LCMS and mass spectrometry results for Example 4a and Example 4b.

### Example 4a: Formation of Zr(IV)-DFOB-L-LYS-DOTA (Zr-3)

To 249 μL of 2.5 mM solution of DFOB-L-LYS-DOTA (3) in ammonium acetate solution (0.2 M, pH 8) was added 62 μL of 50 mM solution of ZrCl₄ in water. The resultant 5:1 solution of Zr(IV):DFOB-L-LYS-DOTA (3) was left to stir at 37° C. for 2 h and then at r.t. for 20 h. The product was analysed via LC-MS. ESI-MS positive ion calculated for C₄₇H₈₃N₁₂O₁₆Zr⁺ [M]⁺: m/z=1161.51. Refer to FIG. 4b and FIG. 5b.

### Example 4b: Formation of Lu(III)-DFOB-L-LYS-DOTA (Lu-3)

To 249 μL of 2.5 mM solution of DFOB-L-LYS-DOTA (3) in ammonium acetate solution (0.2 M, pH 8) was added 62 μL of 50 mM solution of LuCl₃ in water. The resultant 5:1 solution of Lu(III):DFOB-L-LYS-DOTA (3) was left to stir at 37° C. for 2 h and then at r.t. for 20 h. The product was analysed via LC-MS. ESI-MS positive ion calculated for C₄₇H₈₃N₁₂O₁₆Lu [M]: [M+H]⁺ 1247.55. Refer to FIG. 4c and FIG. 5c.

### Example 5

### Synthesis of the Four-Component System DFOB-PPH-L-LYS-DOTA (4)

The synthesis of an embodiment of DFOB-PPH-L-LYS-DOTA (4), is detailed below.

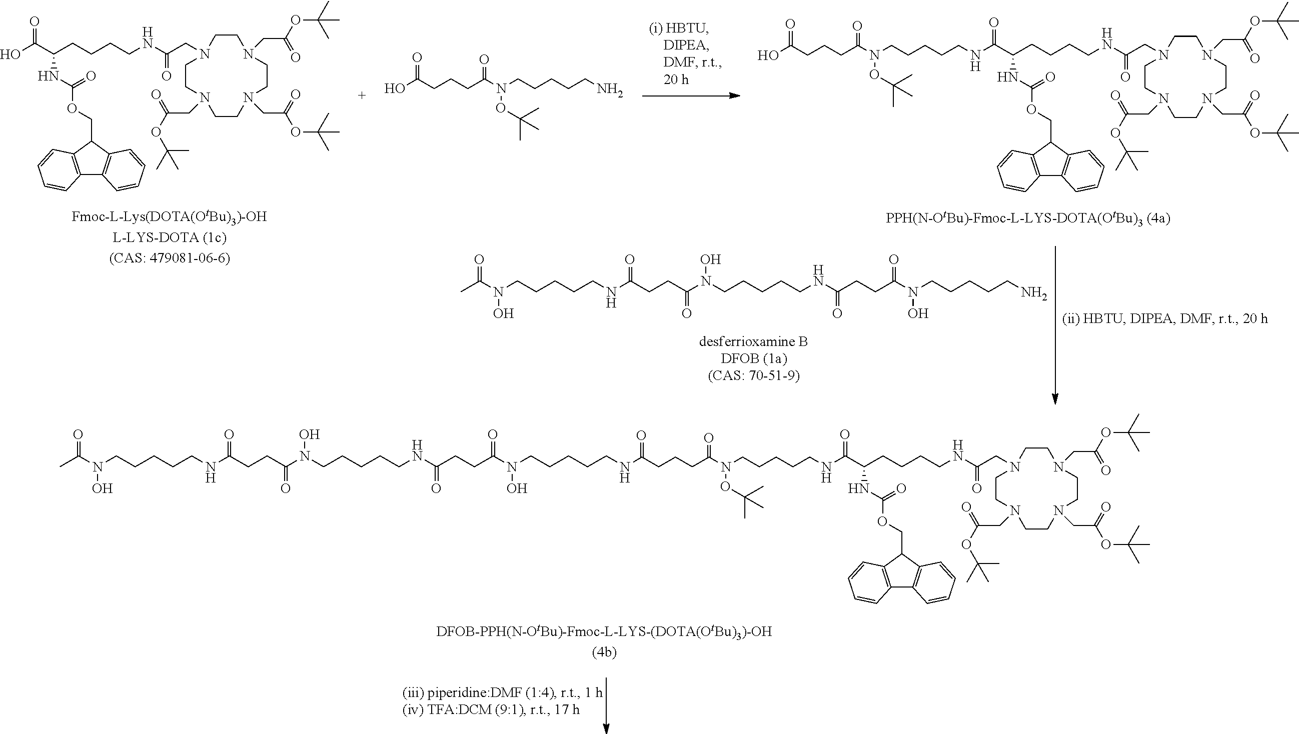

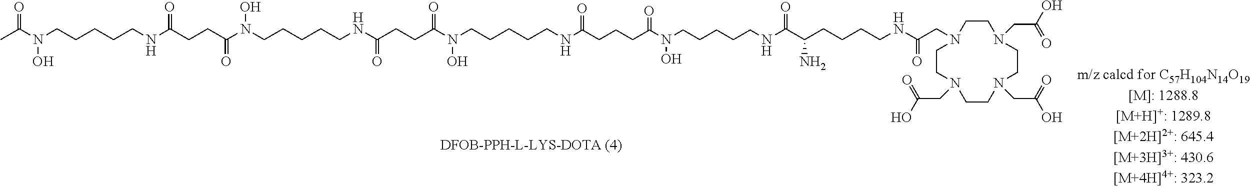 [SMILES: CC(=O)N(O)CCCCCNC(=O)CCC(=O)N(O)CCCCCNC(=O)CCC(=O)N(O)CCCCCNC(=O)CCCC(=O)N(O)CCCCCNC(=O)[C@@H](N)CCCCNC(=O)CN1CCN(CC(=O)O)CCN(CC(=O)O)CCN(CC(=O)O)CC1]

### PPH(N—(Bu)-Fmoc-L-LYS-(DOTA((O^{t}Bu)a)-OH (4a)

A sample of Fmoc-L-Lys(DOTA(O^{t}Bu)₃)—OH (1c) (L-LYS-DOTA, CAS: 479081-06-6) (102.3 mg, 110.9 μmol) was dissolved in dimethylformamide (DMF) (2 mL) followed by the addition of N,N,N′,N′-tetramethyl-O-(1H-benzotriazol-1-yl)uronium hexafluorophosphate (HBTU) (49.3 mg, 130 μmol). The solution was stirred at r.t. for 10 min followed by the addition of diisopropylethylamine (DIPEA) (38.6 μL, 221.6 μmol). The solution was stirred at r.t. for 30 min. A sample of 5-((5-aminopentyl)(tert-butoxy)amino)-5-oxopentanoic acid PPH(N-O^{t}Bu) (39.3 mg, 136.4 μmol) prepared from the treatment of 5-(tert-butoxy(5-((tert-butoxycarbonyl)amino)pentyl)amino)-5-oxopentanoic acid (CAS: 2334242-42-9) with TFA (1:9) was added to the solution, which was stirred at r.t. for 20 h. A volume of DCM (20 mL) was added to the reaction mixture and the mixture was extracted 3 times with 10 mL aliquots of saturated sodium bicarbonate. The organic layer containing the product was dried with anhydrous sodium sulfate and after vacuum filtration, the solvent was removed in vacuo to yield a residue, which was dissolved in DCM and semi-purified using automated flash chromatography (Grace Reverleris X2, 30 mL/min, 12 g cartridge, 0-0.4 min at 5:95 DCM:MeOH, 0.4-7.7 min 5-28% DCM, 7.3-12.68 min at 28% DCM, 12.68-13.41 min at 28-100% DCM, 13.41-15.60 at 100% DCM). The solvent was removed from the collected fractions in vacuo to yield semi-pure PPH(N-MBu)-Fmoc-L-LYS-(DOTA(O^{t}Bu)₃)—OH (4a).

DFOB-PPH(N-O^{t}Bu)-Fmoc-L-LYS-(DOTA(O^{t}Bu)s)-OH (4b)

A sample of PPH(N-O^{t}Bu)-Fmoc-L-LYS-(DOTA(O^{t}Bu)₃)—OH (4a) (167.4 mg, 140.3 μmol) was dissolved in dimethylformamide (DMF) (2 mL) followed by the addition of N,N,N′,N′-tetramethyl-O-(1H-benzotriazol-1-yl)uronium hexafluorophosphate (HBTU) (69.5 mg, 183.3 μmol). The solution was stirred at r.t. for 10 min followed by the addition of diisopropylethylamine (DIPEA) (49 μL, 281.3 μmol). The solution was stirred at r.t. for 30 min followed by the addition of desferrioxamine B mesylate (1a) (117.8 mg, 179.4 μmol) and the solution was stirred at r.t. for 24 h. A volume of DCM (20 mL) was added to the reaction mixture and the mixture was extracted 3 times with 10 mL aliquots of saturated sodium bicarbonate. The organic layer containing the product was dried with anhydrous sodium sulfate and after vacuum filtration, the solvent was removed in vacuo to yield a product that due to the reaction conditions was found to be the Fmoc-deprotected analogue of DFOB-PPH(N-O^{t}Bu)-Fmoc-L-LYS-(DOTA(O^{t}Bu)₃)-OH (4b).

### DFOB-PPH-L-LYS-DOTA (4)

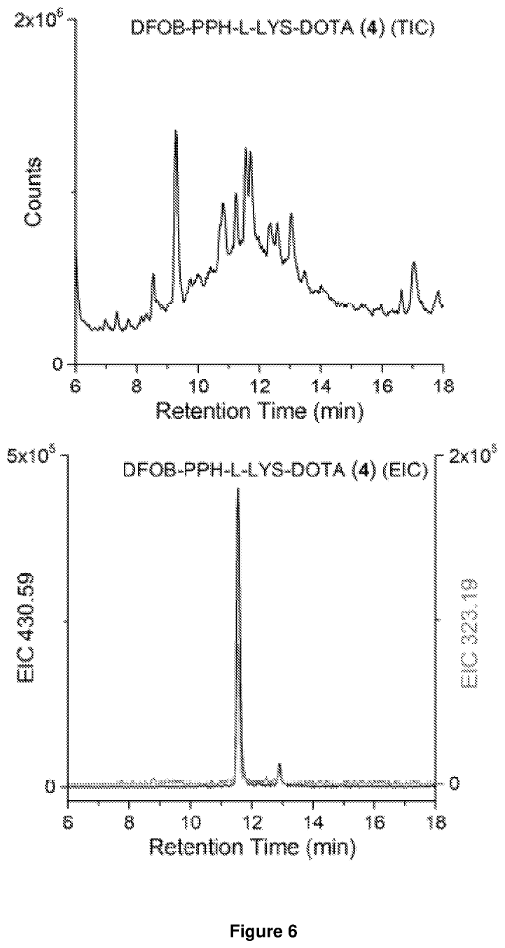

A sample of the Fmoc-deprotected analogue of DFOB-PPH(N-O^{t}Bu)-Fmoc-L-LYS-(DOTA(O^{t}Bu)₃)—OH (4b) (34.9 mg, 23.1 mg) was dissolved in TFA:DCM (9:1, 450 μL:50 μL) with stirring at r.t. for 24 h. The solvent was removed in vacuo and residual TFA was removed by successive treatment (dissolution/in vacuo removal) with methanol and then toluene. The oily residue was suspended in 200 μL of water and the pH of the solution was adjusted to 7 with aliquots of 1 M NaOH or HCl. The solvent was removed in vacuo to give DFOB-PPH-L-LYS-DOTA (4) as a pale yellow solid. Refer to FIG. 6.

### Metal Complexation of DFOB-PPH-L-LYS-DOTA (4)

The inventors expect that DFOB-PPH-L-LYS-DOTA (4) will selectively load metal ions such as Zr(IV) and Lu(III) based upon the results observed with DFOB-L-LYS-DOTA (3) and DFOB-DOTA (2) (see Example 2 and Example 4).

### Example 6

### Synthesis of Compounds Comprising Various Moieties Enhancing ⁸⁹Zr Binding

The inventors expect that the Monomers such as PPH-^{N}O, PPH-^{C}S and PPH—^{N}O^{C}S could be used to replace PPH in the four-component system using the synthetic route described in Example 5. Monomers such as PPH-^{C}O, PPH-^{N}O^{C}O could be used to replace PPH in a synthetic route modified from that described in Example 5 that would further include a reductive deprotection step in addition to steps (iii) and (iv). This is because PPH-^{C}O, PPH-^{N}O^{C}O require protection as N—O-Bn adducts. Methods of protecting group installation and removal are well known in the art.

The inventors further expect that alternative four component systems, such as Compounds 4a-c depicted below, will selectively load metal ions such as Zr(IV) and Lu(III) based upon the results observed with DFOB-L-LYS-DOTA (3) and DFOB-DOTA (2) (see Example 2 and Example 4).

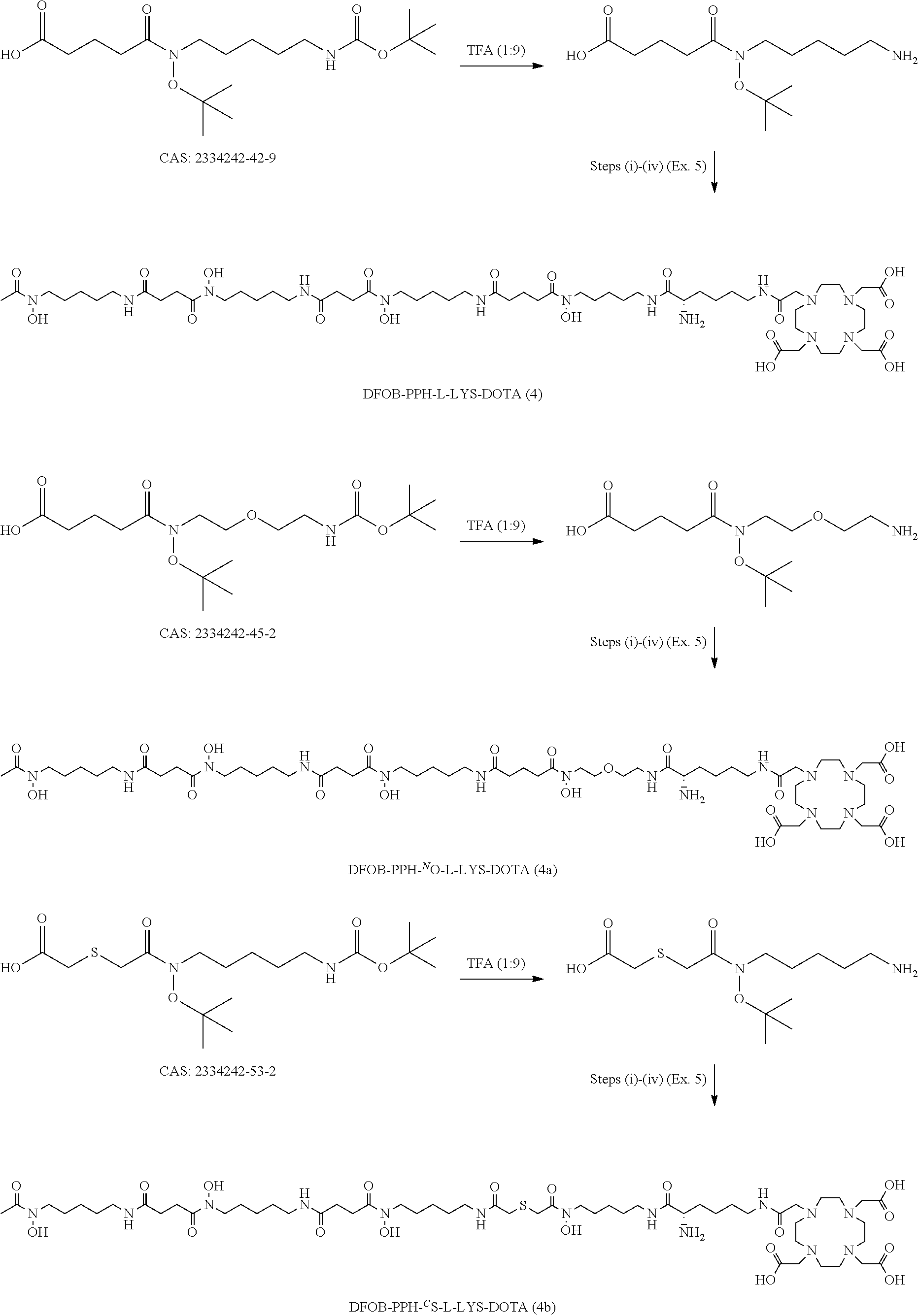

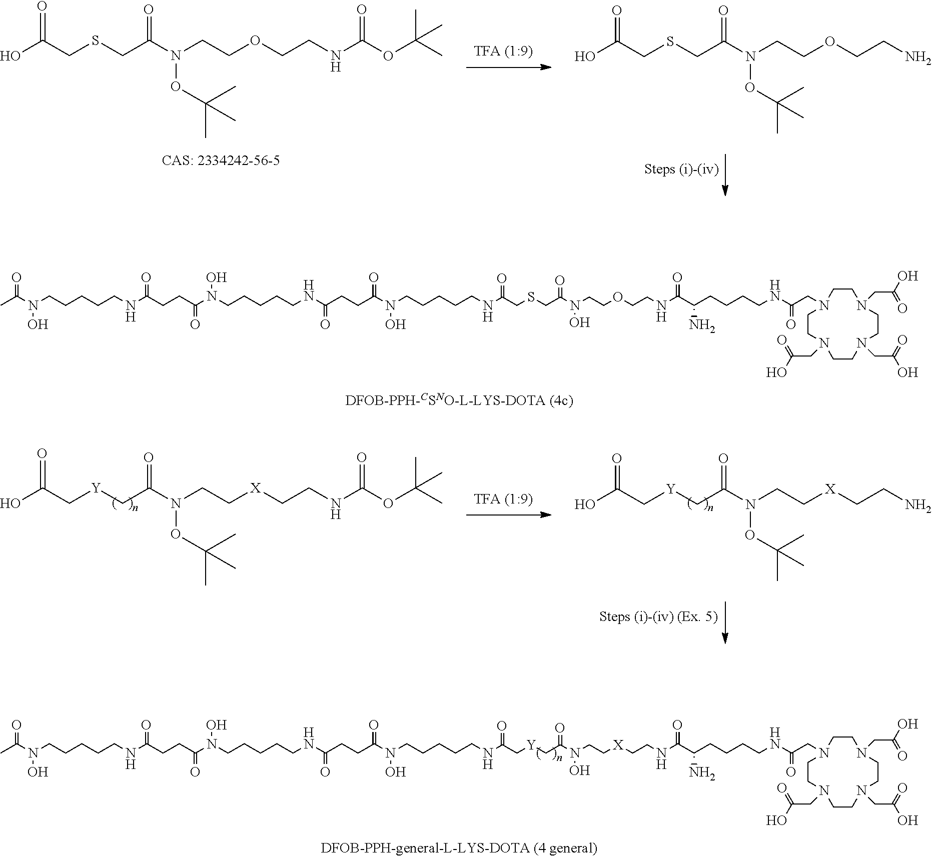

### Example 7

### Synthesis of Systems Comprising Reverse-Hydroxamic Acids

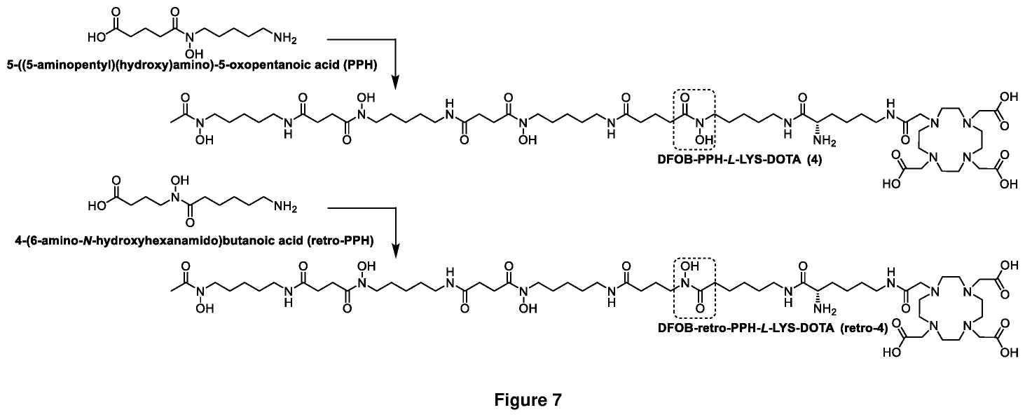

The systems detailed in Example 6 feature ‘forward’ hydroxamic acid monomers as the moiety to enhance ⁸⁹Zr affinity of the chelating ligand selective for ⁸⁹Zr. Alternative systems could employ a reverse-hydroxamic acid as the moiety to enhance ⁸⁹Zr affinity of the chelating ligand selective for ⁸⁹Zr. Reverse-hydroxamic acids are also known as retro-hydroxamic acids in the art. Methods to prepare reverse-hydroxamic acids are known in the art (for instance, see Lifa et al (2015) Inorg. Chem. 54, 3573-3583, Tieu et al (2017) Inorg. Chem. 56, 3719-3728 and Sresutharsan et al (2017) J. Inorg. Biochem. 177, 344-351). Reverse-hydoxamic acids could be incorporated into the compound of the invention through conditions the same or similar to those described in Example 5 and Example 6. The structure of the forward hydroxamic acid 5-((5-aminopentyl)(hydroxy)amino)-5-oxopentanoic acid (PPH) and the corresponding system DFOB-PPH-L-LYS-DOTA (4) is shown in FIG. 7, together with the equivalent reverse-hydroxamic acid 4-(6-amino-N-hydroxyhexanamido)butanoic acid (retro-PPH) and the cognate four-component system DFOB-retro-PPH-L-LYS-DOTA (retro-4).

### Example 8

### Synthesis of DFOB-PPH-DOTA

DFOB-PPH-DOTA may be prepared using methods known in the art, for example, following an analogous route to that described above in Example 5 for DFOB-PPH-L-LYS-DOTA (4). Accordingly, DFOB-PHB-DOTA may be prepared according to the following scheme.

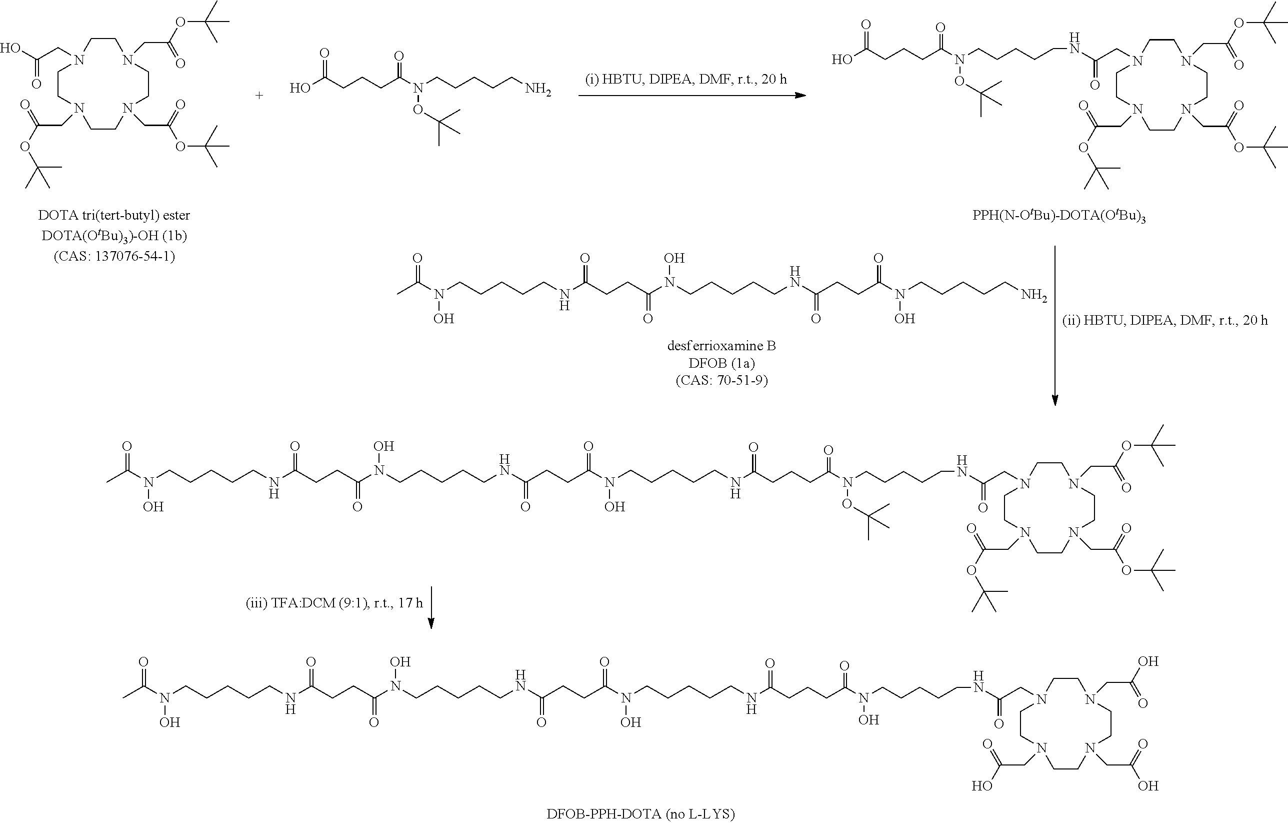

### Example 9

### Synthesis of DFOB-L-LYS(NCS)-DOTA

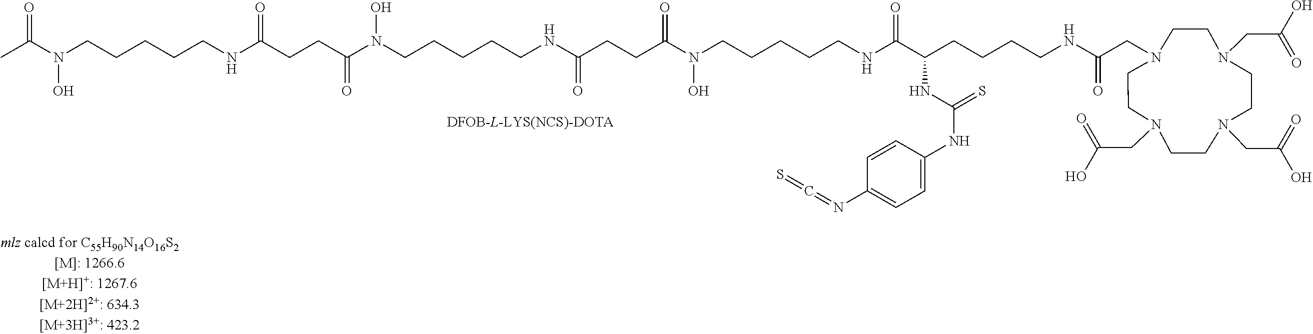 [SMILES: CC(=O)N(O)CCCCCNC(=O)CCC(=O)N(O)CCCCCNC(=O)CCC(=O)N(O)CCCCCNC(=O)[C@H](CCCCNC(=O)CN1CCN(CC(=O)O)CCN(CC(=O)O)CCN(CC(=O)O)CC1)NC(=S)Nc1ccc(N=C=S)cc1]

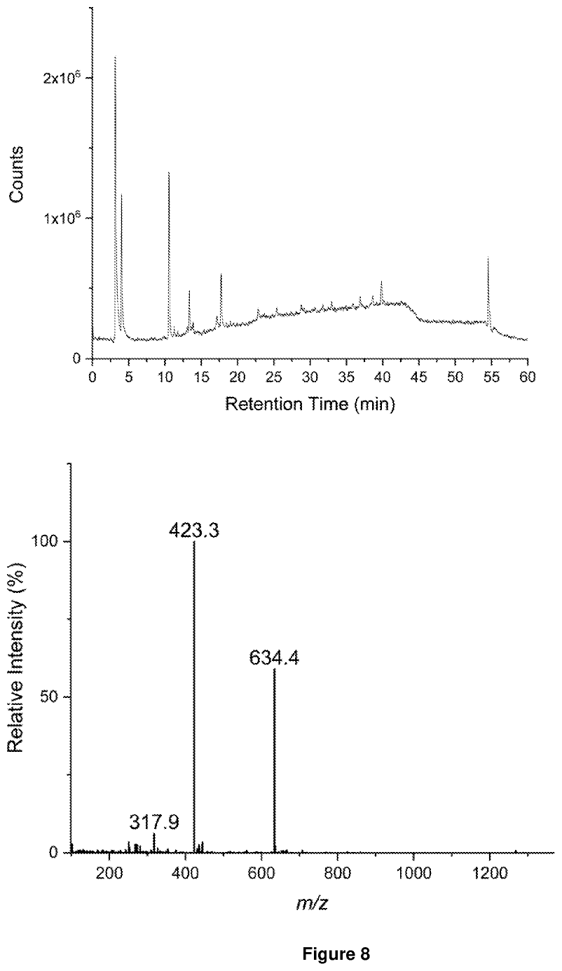

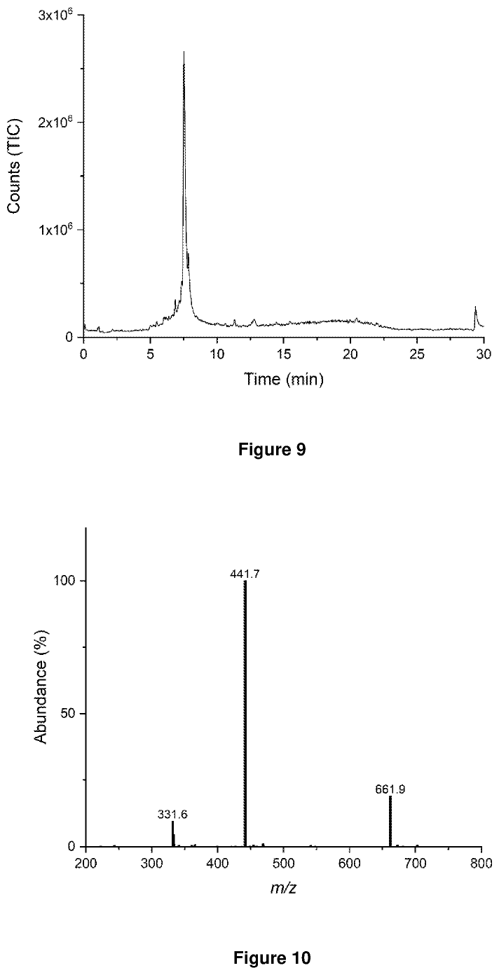

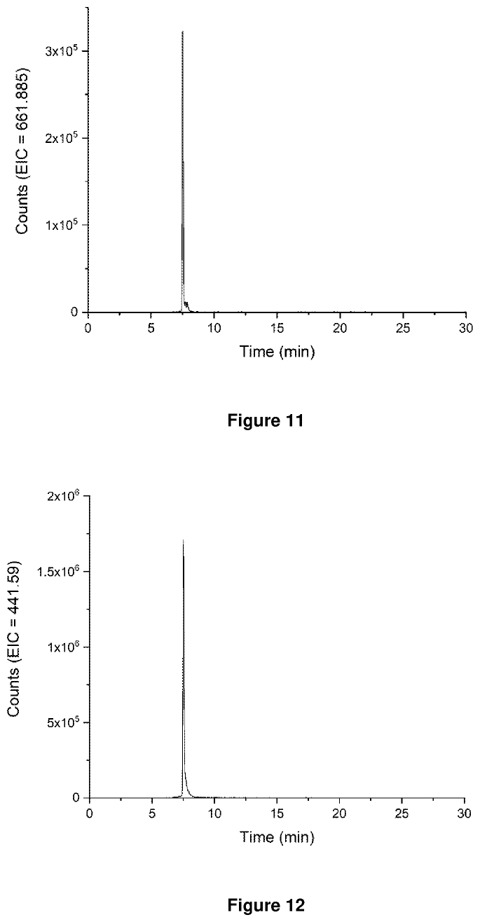

A sample of DFOB-L-LYS-DOTA (5 mg, 4.65 μmol) was dissolved in a solution of isopropanol:water (3.8:1, 316 μL:104 μL) followed by addition of p-phenylene-diisothiocyanate (9.2 mg, 47.9 μmol) in chloroform (500 μL) and triethylamine (8.28 μL, 46.5 μmol). The solution was stirred at room temperature r.t for 21.5 h. The chloroform was removed under vacuum before centrifugation of the remaining solution to isolate the supernatant. The supernatant was removed under vacuum to yield the semi-pure product as a white powder. Refer to FIG. 8.

The inventors expect that this compound will be amenable to conjugation to a targeting moiety, such as an antibody. The inventors further expect that this compound, whether conjugated to a targeting moiety or not, will selectively load metal ions such as Zr(IV) and Lu(III) based upon the results observed with DFOB-L-LYS-DOTA (3) and DFOB-DOTA (2) (see Example 2 and Example 4).

### Example 10

The compound (DFOB-L-LYS-EPS-PEG4-DOTA) has been prepared, as detailed below. The inventors further expect that the free amine group on this PEG group will be amenable to further chemistry to enable conjugation to a targeting moiety.

### Synthesis of N-Boc-Amino-Acid-PEG4.

Amino-Acid-PEG4 (121.5 mg, 0.46 mmol) and sodium hydroxide (23.4 mg, 0.59 mmol) were dissolved in a mixture of dioxane-water (2:1, 866:434 μL). The solution was cooled to 0° C. before the dropwise addition of Boc₂O (166.1 mg, 0.76 mmol) in a mixture of dioxane:water (2:1, 176.9:88.6 μL). The solution was stirred at room temperature for 21 h. Upon completion, the solvent was removed in vacuo before dissolving the residue in water (20 mL). The residue was washed with ethyl acetate (3×12 mL) before adjusting the aqueous phase to pH 1-2 with 1 M HCl and extracting further with ethyl acetate (3×20 mL). The organic fractions were combined, dried over anhydrous magnesium sulphate and filtered before removing the solvent in vacuo to yield N-Boc-Amino-Acid-PEG4 as a colourless oil.

### Synthesis of DFOB-L-LYS-EPS-N-Boc-PEG4-DOTA (O^{t}Bu)s.

N-Boc-Amino-Acid-PEG4 (14.9 mg approx., 0.041 mmol) was dissolved in DMF (1 mL) before adding DIPEA (10.4 μL, 0.060 mmol) and the reaction stirred at room temperature for 10 min. HBTU (15.4 mg, 0.041 mmol) was added to the solution, which was stirred at room temperature for 30 min. DFOB-L-LYS-EPS-DOTA (O^{t}Bu)₃ (44.5 mg approx., 0.036 mmol) in DMF (5 mL) was added and the solution stirred at room temperature for 2 h. Upon completion, the reaction solution was diluted with DCM (50 mL) and extracted with saturated sodium bicarbonate (3×25 mL) and brine (1×25 mL). The organic layer was dried over anhydrous magnesium sulphate before removing the solvent in vacuo to give DFOB-L-LYS-EPS-N-Boc-PEG4-DOTA (OtBu)₃ as a yellow oil (28.5 mg, 46.9%).

### Synthesis of DFOB-L-LYS-EPS-PEG4-DOTA.

DFOB-L-LYS-EPS-N-Boc-PEG4-DOTA (OtBu)₃ (28.5 mg, 0.019 mmol) was dissolved in a solution of TFA:DCM (9:1, 2 mL) and stirred at room temperature for 18 h. Upon completion, the solvent was removed in vacuo. The residue was dissolved in methanol (5 mL) and the solvent was removed in vacuo, before repeating with toluene (5 mL). The residue was neutralised to pH 7 using 1 M NaOH and 1 M HCl to give DFOB-L-LYS-EPS-PEG4-DOTA as a pale-yellow solid.

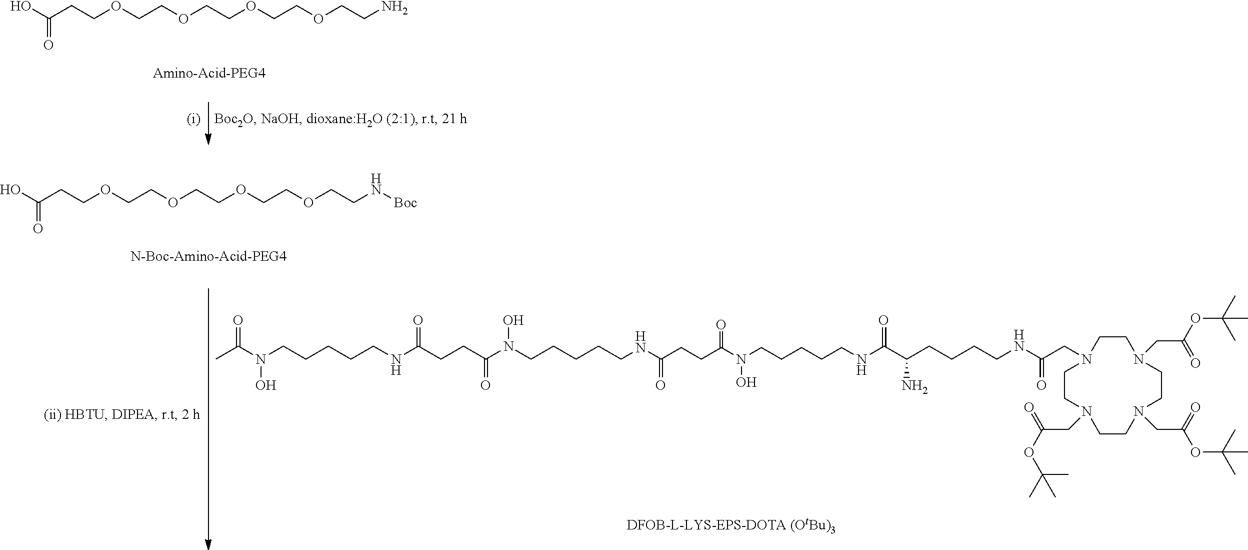 [SMILES: NCCOCCOCCOCCOCCC(=O)O ; CC(C)(C)OC(=O)NCCOCCOCCOCCOCCC(=O)O ; CC(=O)N(O)CCCCCNC(=O)CCC(=O)N(O)CCCCCNC(=O)CCC(=O)N(O)CCCCCNC(=O)[C@@H](N)CCCCNC(=O)CN1CCN(CC(=O)OC(C)(C)C)CCN(CC(=O)OC(C)(C)C)CCN(CC(=O)OC(C)(C)C)CC1]

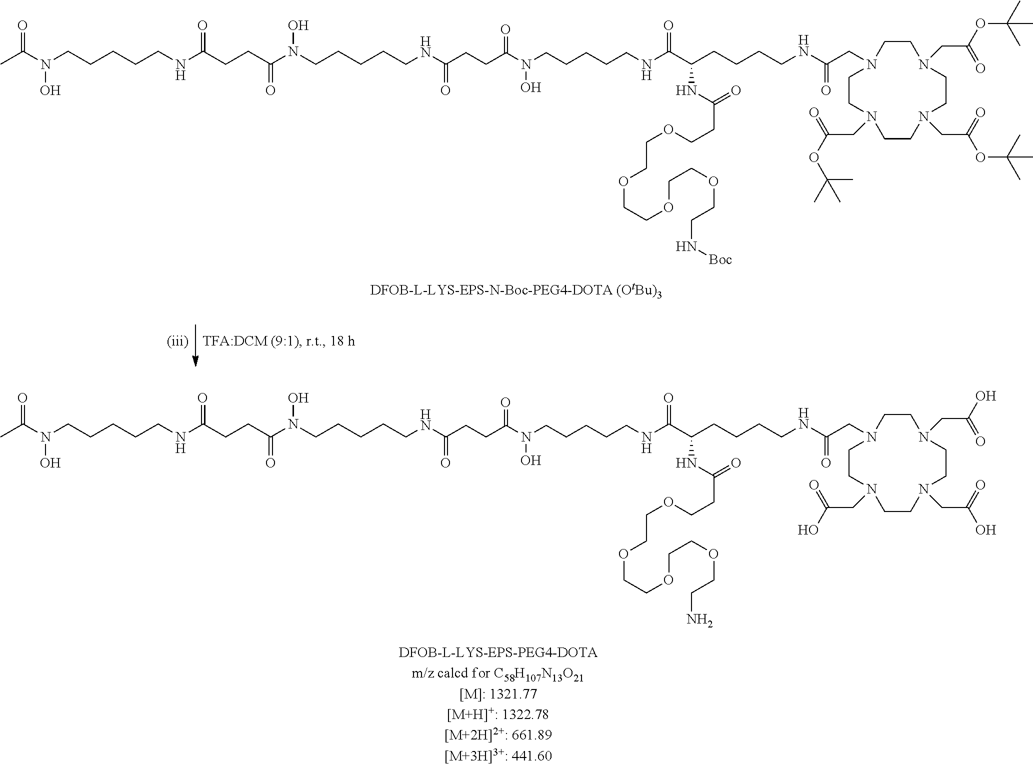

### Synthetic Scheme for DFOB-L-LYS-EPS-PEG4-DOTA

### Example 11

The compound (NCS-Activated-DFOB-L-LYS-EPS-PEG4-DOTA—Compound D2) has been prepared, as detailed below.

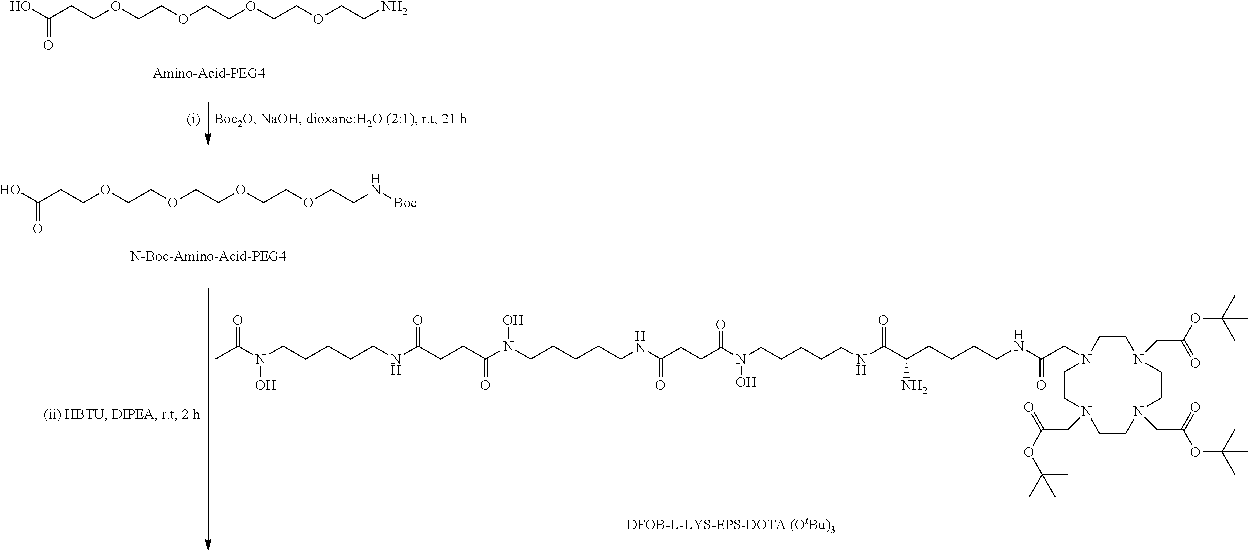 [SMILES: NCCOCCOCCOCCOCCC(=O)O ; CC(C)(C)OC(=O)NCCOCCOCCOCCOCCC(=O)O ; CC(=O)N(O)CCCCCNC(=O)CCC(=O)N(O)CCCCCNC(=O)CCC(=O)N(O)CCCCCNC(=O)[C@@H](N)CCCCNC(=O)CN1CCN(CC(=O)OC(C)(C)C)CCN(CC(=O)OC(C)(C)C)CCN(CC(=O)OC(C)(C)C)CC1]

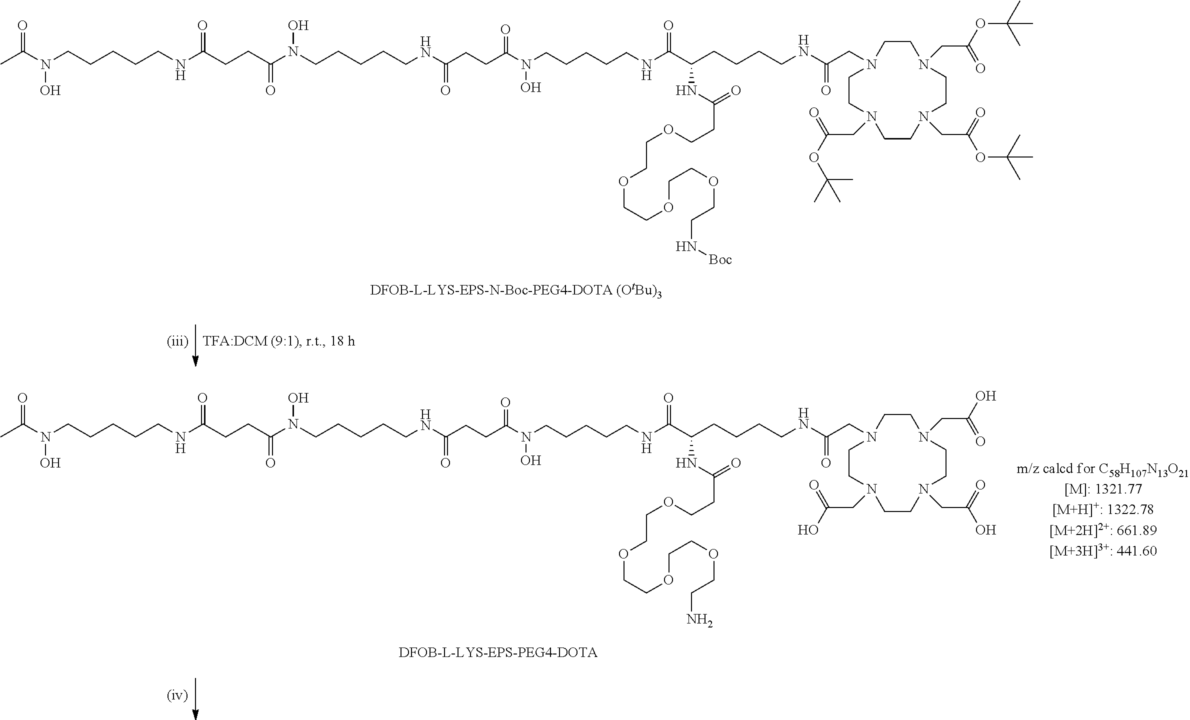

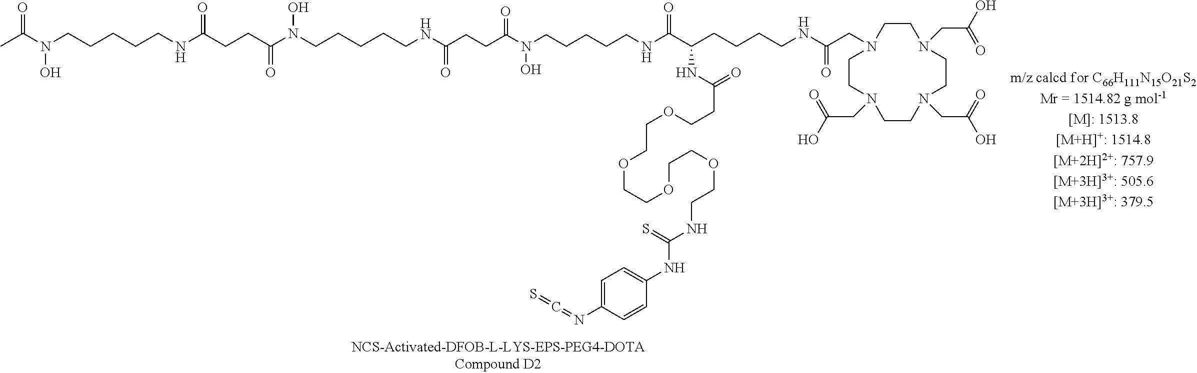 [SMILES: CC(=O)N(O)CCCCCNC(=O)CCC(=O)N(O)CCCCCNC(=O)CCC(=O)N(O)CCCCCNC(=O)[C@H](CCCCNC(=O)CN1CCN(CC(=O)O)CCN(CC(=O)O)CCN(CC(=O)O)CC1)NC(=O)CCOCCOCCOCCOCCNC(=S)Nc1ccc(N=C=S)cc1]

### Synthetic Scheme for NCS-Activated DFOB-L-LYS-EPS-PEG4-DOTA (Compound D2)

### Synthesis of L-Fmoc-LYS-EPS-DOTA (OtBu)₃

To a solution of DOTA (OtBu)₃ (1.7677 g, 3.09 mmol) in DMF (50 mL) was added DIPEA (3.6 mL, 20.67 mmol). The mixture was stirred at room temperature for 10 minutes. HBTU (1.1728 g, 3.09 mmol) was then added and the mixture stirred at room temperature for a further 30 minutes. Fmoc-Lys-OH HCl (1.2643 g, 3.43 mmol) was then added, and the resultant mixture stirred at room temperature for a further 2 hours. Upon completion, the solvent was removed in vacuo. The residue was purified via solid phase extraction (Method A). The collected fractions were combined and lyophilised to yield L-Fmoc-Lys-DOTA (OtBu)₃ (1) as a yellow/white solid (1.7402 g, 1.63 mmol, 52.8%).

### Synthesis of DFOB-L-Fmoc-LYS-EPS-DOTA (OtBu)₃

To a solution of L-Fmoc-LYS-EPS-DOTA (OtBu)₃ (595.3 mg, 0.65 mmol) in DMF (50 mL) was added DIPEA (194 μL, 1.11 mmol). The mixture was stirred at room temperature for 10 minutes. HBTU (258.2 mg, 1.10 mmol) was then added, and the resultant mixture stirred at room temperature for a further 30 minutes. Desferoxamine B mesylate (444.1 mg, 0.67 mmol) was then added and the resultant mixture stirred at 50° C. for a further hour. Upon completion, the reaction solution was removed in vacuo. The resultant residue was diluted with DCM (500 mL) and extracted with saturated sodium bicarbonate (3×250 mL) and saturated brine (1×250 mL). The organic layer was dried over anhydrous magnesium sulphate before removing the solvent in vacuo to give DFOB-L-Fmoc-Lys-EPS-DOTA(OtBu)₃ as a yellow/green oil (698 mg, 0.48 mmol, 70.6%).

### Synthesis of DFOB-L-LYS-EPS-DOTA (OtBu)₃

DFOB-L-Fmoc-Lys-EPS-DOTA(OtBu)₃ (698 mg, 0.48 mmol) was dissolved in a solution of piperidine:DMF (1:4) (1 mL:4 mL) and left to stir at r.t for 1 h. Upon completion, the solvent was removed in vacuo. Diethyl ether (c.a. 5 mL) was then added to the resultant residue and left to stir for 10 minutes before decanting the ether. The solvent was removed in vacuo to yield DFOB-L-Lys-DOTA(OtBu)₃ as a yellow/white crystalline product (539.4 mg, 0.43 mmol, 90%).

### Synthesis of N-Boc-Amino-Acid-PEG4.

Amino-Acid-PEG4 (395.3 mg, 1.49 mmol) and sodium hydroxide (84.7 mg, 2.12 mmol) were dissolved in a mixture of dioxane-water (2:1, 4 mL). The solution was cooled to 0° C. before the dropwise addition of Boc₂O (613.9 mg, 2.81 mmol) in a mixture of dioxane:water (2:1, 1 mL). The solution was stirred at room temperature for 18 h. Upon completion, the solvent was removed in vacuo before dissolving the residue in water (20 mL). The residue was washed with ethyl acetate (3×12 mL) before adjusting the aqueous phase to pH 1-2 with 1 M HCl and extracting further with ethyl acetate (3×20 mL). The organic fractions were combined, dried over anhydrous magnesium sulphate and filtered before removing the solvent in vacuo to yield N-Boc-Amino-Acid-PEG4 as a pale yellow oil (387.5 mg, 1.06 mmol, 71.1%).

### Synthesis of DFOB-L-LYS-EPS-N-Boc-PEG4-DOTA (OtBu)₃.

N-Boc-Amino-Acid-PEG4 (118.8 mg, 0.33 mmol) was dissolved in DMF (50 mL) before adding DIPEA (113.4 μL, 0.65 mmol) and the reaction stirred at room temperature for 10 min. HBTU (149.5 mg, 0.64 mmol) was added to the solution, which was stirred at room temperature for 30 min. DFOB-L-LYS-EPS-DOTA (OtBu)₃ (539.4 mg, 0.43 mmol) was added and the solution stirred at room temperature for approx. 18.5 h. Upon completion, the reaction solution was removed in vacuo. The resultant residue was diluted with DCM (100 mL) and extracted with saturated sodium bicarbonate (3×50 mL) and saturated brine (1×50 mL. The organic layer was dried over anhydrous magnesium sulphate before removing the solvent in vacuo to give DFOB-L-LYS-EPS-N-Boc-PEG4-DOTA (OtBu)₃ as a yellow oil (465 mg, 0.29 mmol, 87.9%).

### Synthesis of DFOB-L-LYS-EPS-PEG4-DOTA.

DFOB-L-LYS-EPS-N-Boc-PEG4-DOTA (OtBu)₃ (465 mg, 0.29 mmol) was dissolved in a solution of TFA:DCM (9:1, 5 mL) and stirred at room temperature for approx. 19 h. Upon completion, the solvent was removed in vacuo. The residue was dissolved in toluene (10 mL) and rinsed with minimal methanol to transfer from vial, before removing solvent in vacuo. The resultant residue was purified via SPE (Method B) to yield DFOB-L-LYS-EPS-PEG4-DOTA as a yellow oil (115.7 mg, 0.09 mmol, 31.0%).

### Synthesis of NCS-Activated-L-LYS-EPS-PEG4-DOTA.

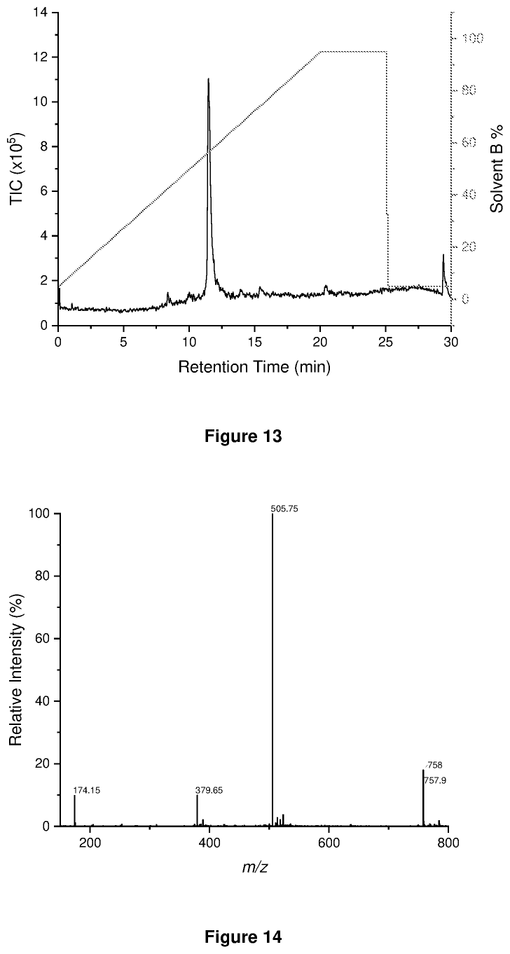

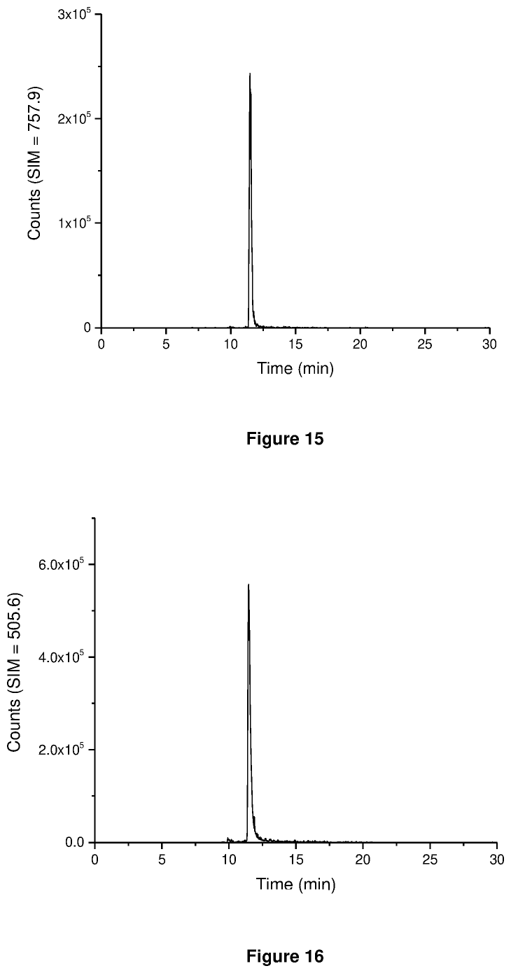

This synthetic step was run as 2 reactions in parallel. In an Eppendorf tube, DFOB-L-LYS-EPS-PEG4-DOTA (13.2 mg, 10.2 mg, 0.018 mmol total) was dissolved in DMF (1 mL, 1 mL) and triethylamine (28 μL, 22 μL, 0.29 mmol total) was added to the solution. In a separate Eppendorf tube, a solution of 1,4-phenylenediisothiocyanate (22.3 mg, 17.7 mg, 0.250 mmol total) in DMF (920 μL, 484 μL) was prepared. The solution of DFOB-L-LYS-EPS-PEG4-DOTA was added to the 1,4-phenylenediisothiocyanate solution in 5 aliquots (5×206 μL, 5×204 μL), pulsing to combine between additions. Each reaction was split into an additional Eppendorf tube (4 total). The resultant mixtures were then centrifuged at 800 rpm for 1.5 h. Upon completion, the resultant solution was split across a further 5 Eppendorf tubes (162 μL, 126 μL per tube, 24 tubes total), diethyl ether (c.a 508 μL, 392 μL each) was added, and the resultant solutions were stored in a refrigerator for 2 h. The resultant solutions were then centrifuged at 12000 rpm for 3 minutes, before decanting the ether. The pellets were then washed with cold diethyl ether (c.a. 564 μL, 436 μL each) and air dried. The pellets were then dissolved in DMF (c.a. 22.5 μL, 17.4 μL each) and MeOH (c.a. 169 μL, 131 μL each), before addition of diethyl ether (c.a. 508 μL, 392 μL each) and refrigeration for another approx. 23 h. Upon completion, the resultant solutions were centrifuged at 12000 rpm for 3 minutes, before decanting the ether. The pellets were then washed with further cold diethyl ether (c.a. 564 μL, 436 μL each) and air dried. The pellets were then transferred to a vial with methanol and dried in vacuo to yield crude NCS-Activated-DFOB-L-LYS-EPS-PEG4-DOTA (Compound D2) as a yellow/white oil. HPLC purification was performed on the crude material and the collected fractions lyophilised to yield pure Compound D2 as a white solid (2.9 mg, 0.002 mmol, 11.1%). This compound was characterised using LC-MS (FIGS. 13 to 16) and was >95% pure based on this method (FIG. 13).

### High Performance Liquid Chromatography

Preparative high-performance liquid chromatography (HPLC) was conducted on a Shimadzu LC-20 series LC system with two LC-20AP pumps, SIL-10AP autosampler, a SPD-20A UV/VIS detector, and an FRC-10A fraction collector. A Shimadzu Shimpack GIS column (150×10 mm i.d., 5 mL min⁻¹, particle size 5 μm) was used for semipreparative purification. The organic phase (B) consisted of ACN:TFA (99.95:0.05). The aqueous phase (A) consisted of H₂O:TFA (99.95:0.05). Spectral data was acquired and processed using Shimadzu LabSolutions Software (version 5.98). The method used a gradient of 20-25% solvent B over 5 minutes, 25-45% solvent B over 35 minutes, 20% solvent B for 5 minutes with a flow rate of 5 mL min⁻¹.

### Solid Phase Extraction

Solid phase extraction (SPE) was conducted using Waters SEP-PAK C18 5 g and 2 g vacuum cartridges on a manual vacuum manifold.

### Method A:

The cartridge was conditioned with 1 column volume of ACN, followed by 1 column volume of Milli-Q water. The sample was loaded in 100% Milli-Q water, rinsing any remaining residue from the reaction vessel with minimal ACN. The cartridge was then washed with 1 column volume of Milli-Q water. The cartridge was then subjected to 2 column volumes of 20-55% ACN in Milli-Q water, collecting the 45-55% fractions.

### Method B:

The cartridge was conditioned with 1 column volume of ACN, followed by 1 column volume of Milli-Q water. The sample was loaded in 100% Milli-Q water. The cartridge was then washed with 1 column volume of Milli-Q water. The cartridge was then subjected to 4 column volumes of 80% ACN in Milli-Q water, collecting these fractions.

### Example 12

Conjugation of NCS-Activated DFOB-L-LYS-EPS-PEG4-DOTA (Compound D2 or D2) to Girentuximab 1 mg of NCS-Activated DFOB-L-LYS-EPS-PEG4-DOTA (Compound D2 or D2) (˜20-fold molar excess) dissolved in dimethylsulfoxide (DMSO, 2.5 mg/mL) was added to 6 mg of Girentuximab in 1× phosphate buffered saline (PBS, 10 mg/mL). To this mixture was added 6% v/v of 1M Na₂CO₃ with a final reaction pH of 8.5-9.0. The reaction mixture was allowed to react for 1 hour at 37° C. while being gently agitated at 550 rpm. The resulting mixture was purified using Amicon Spin Membranes (10 kDa MWCO, 0.5 mL), 8-10 times. The purity of the conjugated compound was analysed using HPLC (UV detection at 280 nm) and the chelator to antibody ratio was determined to be 1-2 for each antibody in this study by matrix-assisted laser desorption ionisation —time of flight mass spectrometry (MALDI TOF-MS).

### Example 13

### Radiolabelling of Compound D2-mAb

Compound D2-mAb (where mAb=Girentuximab) was incubated with ⁸⁹Zr at an excess of antibody in buffer for 60 minutes at 37° C. with shaking at 550 rpm. The radioTLC confirmed no unbound ⁸⁹Zr. The compound was spun using a Zeba Spin desalting column (40 kDa MWCO, 2 mL) and the material was determined to be >95% in radiochemical yield and the material was injected into the mouse HT-29 xenograft model for positron emission tomography (PET) imaging. The DFOB-mAb[⁸⁹Zr] control gave a quantitative radiochemical yield. Compound D2-mAb was incubated with ¹⁷⁷Lu at an excess of antibody in 0.1M NH₄OAc (pH 5-6), for 60 minutes at 37° C. with shaking at 400 rpm. The DOTA-mAb[¹⁷⁷Lu] control gave a quantitative radiochemical yield.

### Example 14

In Vitro Studies of Compound D2-mAb[⁸⁹Zr] and Compound D2-mAb[¹⁷⁷Lu], Compared with the Respective Control Compounds DFOB-mAb[⁸⁹Zr] and DOTA-mAb[¹⁷⁷Lu]

### Cell Binding Assays

### Seeding and Incubation

HT-29 cells were seeded at a density of 7.5×10² cells/well in a 24-well plate with a final number of approximately 2.5×10⁵ cells per well. The radiolabelled mAbs (Compound D2-mAb[⁸⁹Zr], Compound D2-mAb[¹⁷⁷Lu], DFOB-mAb[⁸⁹Zr], DOTA-mAb[¹⁷⁷Lu]) were diluted in serum-free (SF) cell growth medium (0.02 μg, 15 mCi) and 100 μL of each solution was added to each well. The cells were incubated in triplicate for 0.5, 1, and 2 hours at 37° C. in a 5% CO₂ atmosphere in a humidified incubator.

### Determining the Membrane-Bound Fraction

At each time point, internalization was halted by removing the growth medium and washing the cells twice with ice-cold PBS (1×, pH 7.4, 200 μL). Receptor-bound radiolabelled mAb was then removed using ice-cold glycine buffer containing 4M urea (0.2 M, pH 2.0, 200 μL) for 5 minutes. The buffer was collected from each well and the radioactivity measured in a gamma counter to determine the membrane-bound fraction. The cells were then washed once with the same glycine buffer.

### Determining the Internalised Fraction

Cells were treated with sodium hydroxide (1N, 200 μL) for 30 minutes to lyse the cells, internalised fractions collected, and the radioactivity measured of the subsequent fractions in a gamma-counter.

### Non-Specific Binding

Cells were counted after the assay from 4 non-experimental wells and the cell numbers were averaged to obtain an estimate of the number of cells per well. Non-specific binding and internalization were then determined by co-incubating cells with non-radiolabelled (2 μg, 50 μL) and radio-labelled (0.02 μg, 50 μL) compound for each well, and repeating the above procedures for membrane-bound and internalised fractions.

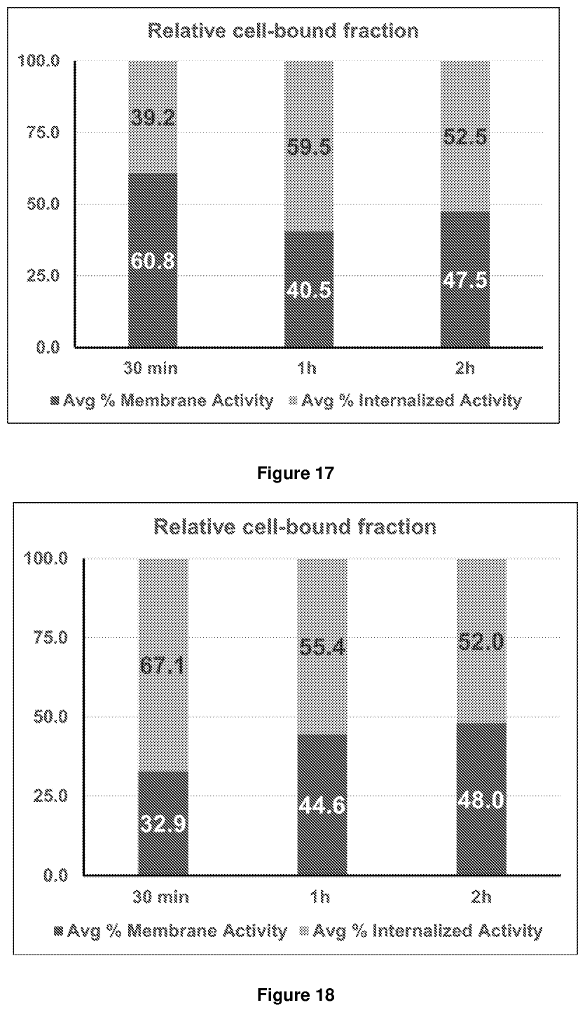

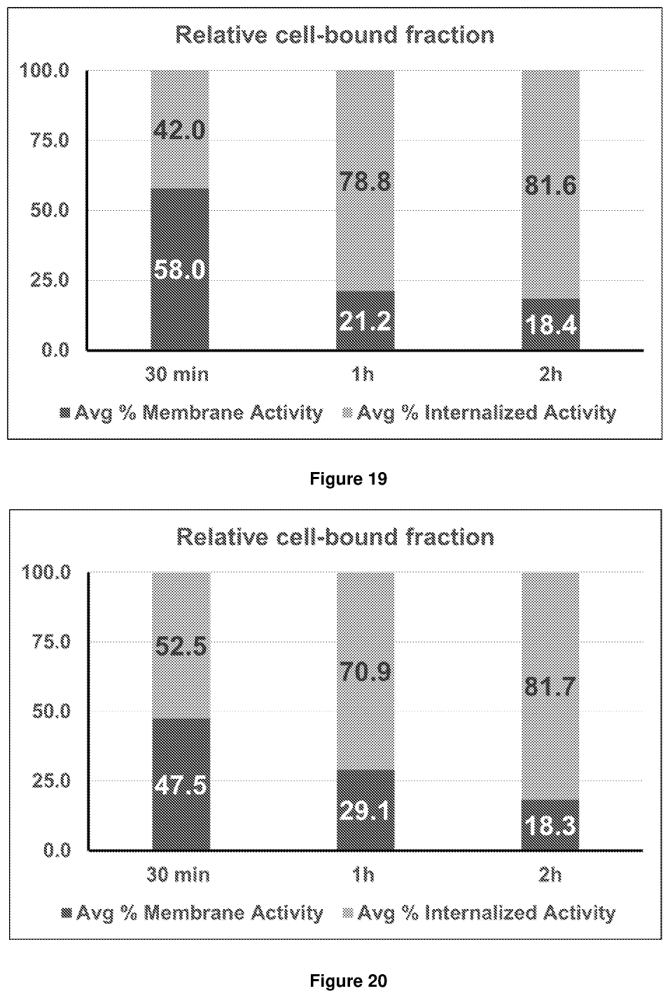

These data showed that the fraction of membrane bound or internalised radiolabelled mAb of Compound D2-mAb[¹⁷⁷Lu] and DOTA-mAb[¹⁷⁷Lu] was similar at 1 h and 2 h (FIGS. 17 and 18); and that the fraction of membrane bound or internalised radiolabelled mAb of Compound D2-mAb[⁸⁹Zr] and DFOB-mAb[⁸⁹Zr] was similar at 1 h and 2 h (FIGS. 19 and 20).

### Example 15

In Vivo and Ex Vivo Studies of Compound D2-mAb[⁸⁹Zr] and Compound D2-mAb[¹⁷⁷Lu], compared with the respective control compounds DFOB-mAb[⁸⁹Zr] and DOTA-mAb[¹⁷⁷Lu]

8-week-old male Balb/c nude mice were injected subcutaneously with HT-29 (10×10⁶) cells in 50 μL of 50:50 matrigel and cells in phosphate buffered saline into the right flank of each mouse. Labelled mAbs (Compound D2-mAb[⁸⁹Zr] or DFOB-mAb[⁸⁹Zr]) were injected via the tail vein (29G needle; ˜1-4 MBq) and then mice were imaged using the Siemens Inveon PET-CT instrument at the various timepoints for ⁸⁹Zr and were sacrificed at 48 hours and their harvested organs counted via gamma counting.

### In Vivo and Ex Vivo Biodistribution

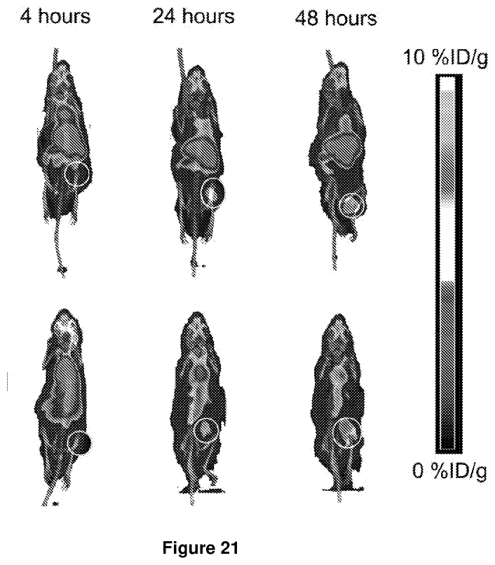

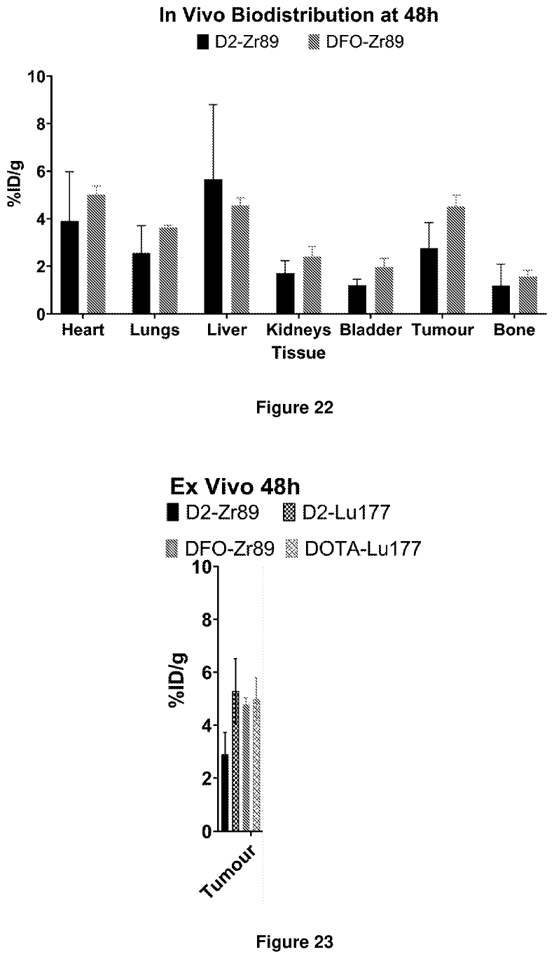

Mice injected with ⁸⁹Zr labelled mAbs were imaged at 4 hours, 24 hours and 48 hours post-injection (FIG. 21) and in vivo biodistribution measured at 48 h (FIG. 22). At 48 hours post-injection organs were harvested for gamma counting and quantification of organ distribution for both ⁸⁹Zr and ¹⁷⁷Lu injected mice (FIG. 23).

### Example 16

Preparation of Compound D2-mAb[^{nat}Lu][⁸⁹Zr] and Compound D2-mAb[¹⁷⁷Lu][^{nat}Zr]

As shown in the scheme below, compound D2 can be loaded with non-toxic natural Lu(III) to produce Compound D2[^{nat}Lu] which is subsequently conjugated to an mAb to produce Compound D2-mAb[^{nat}Lu] for radiolabelling with ⁸⁹Zr to produce Compound D2-mAb[^{nat}Lu][⁸⁹Zr] for immunological PET imaging.

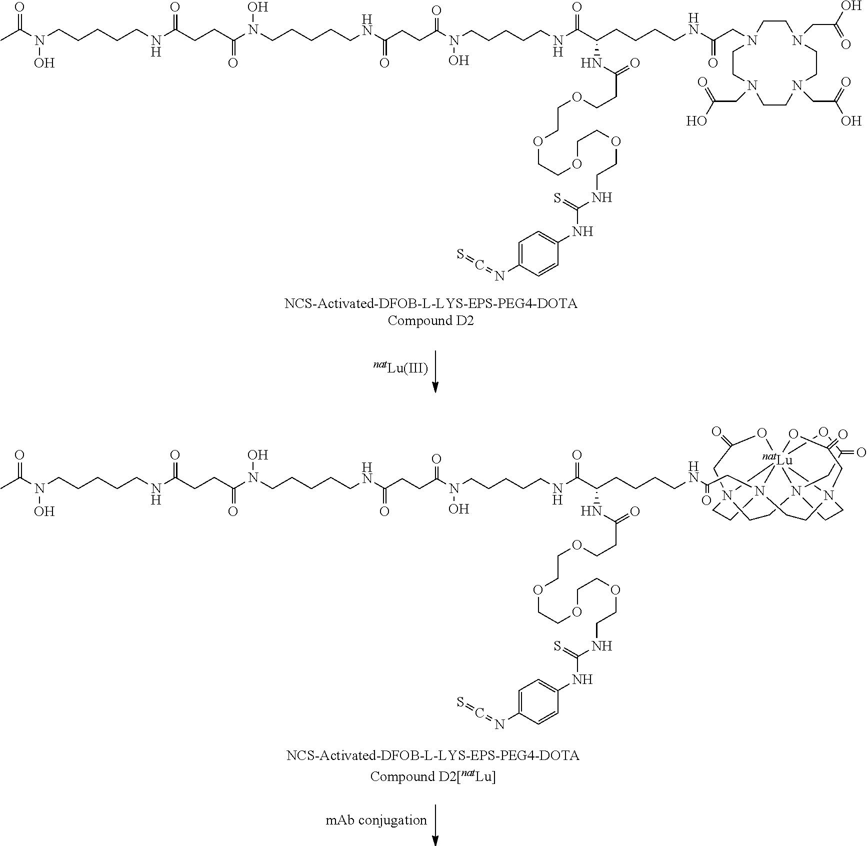

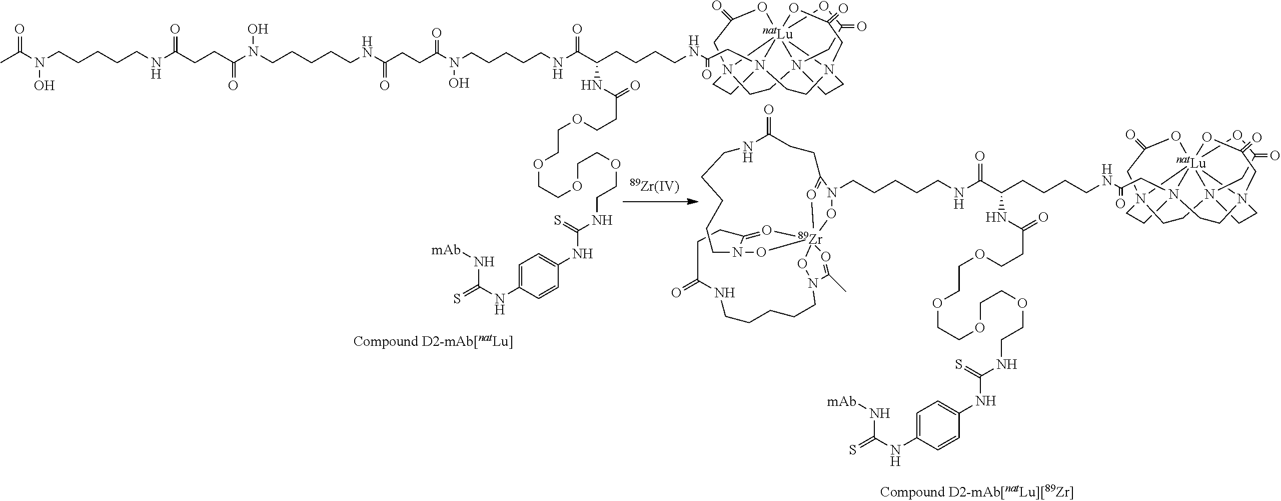

Compound D2-mAb[^{nat}Lu][⁸⁹Zr] would be prepared by incubating Compound D2 with natural Lu(III) which would bind to the DOTA region of Compound D2. Compound D2[^{nat}Lu] would be conjugated to an mAb to generate Compound D2-mAb[^{nat}Lu] and this compound would be radiolabelled with 89Zr to generate Compound D2-mAb[^{nat}Lu][⁸⁹Zr] useful for immunological PET imaging.

In a similar manner, as shown in the scheme below, Compound D2 can be loaded with non-toxic natural Zr(IV) to produce Compound D2[^{nat}Zr] which is subsequently conjugated to an mAb to produce Compound D2-mAb[^{nat}Zr] for radiolabelling with ¹⁷⁷Lu to produce Compound D2-mAb[¹⁷⁷Lu][^{nat}Zr] for therapy.

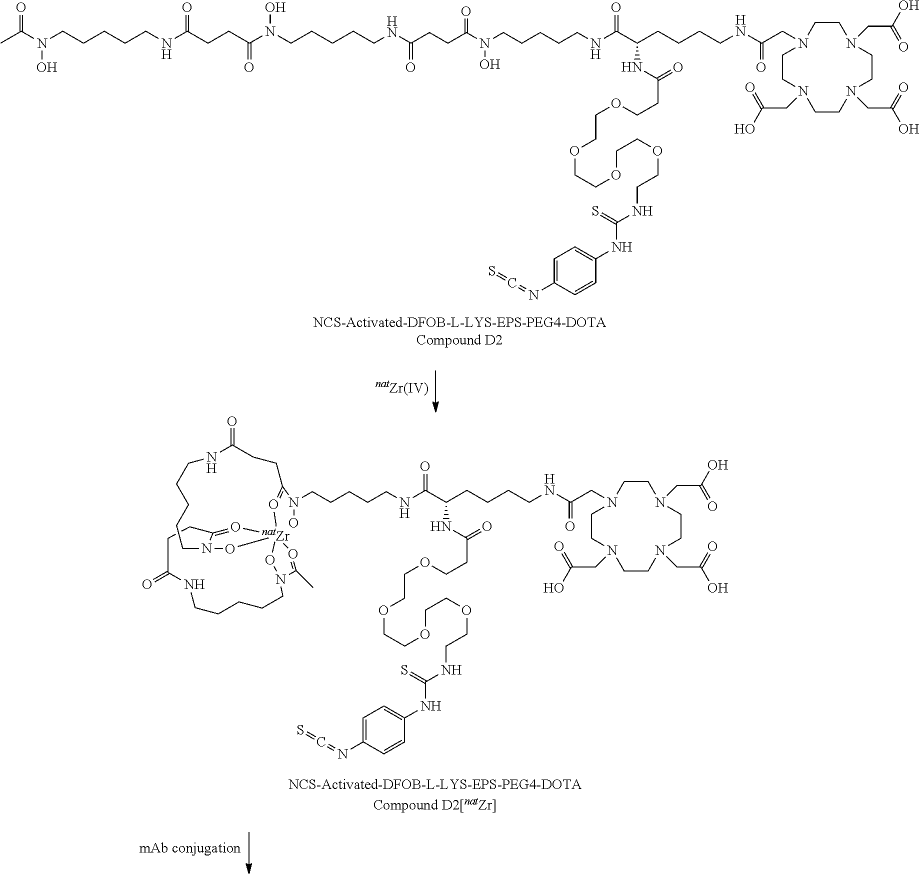

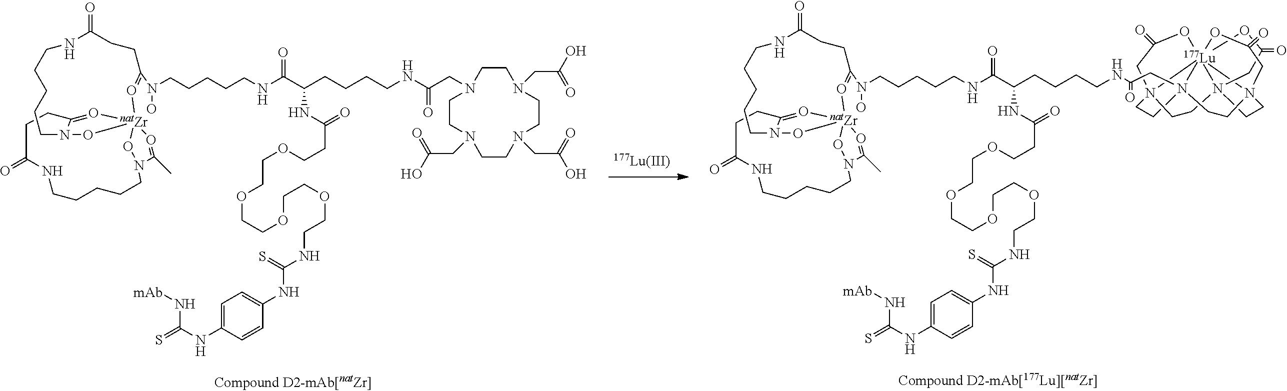

Compound D2-mAb[¹⁷⁷Lu][^{nat}Zr] would be prepared by incubating Compound D2 with natural Zr(IV) which would bind to the DFOB region of Compound D2. Compound D2[^{nat}Zr] would be conjugated to an mAb to generate Compound D2-mAb[^{nat}Zr] and this compound would be radiolabelled with 177Lu to generate Compound D2-mAb[¹⁷⁷Lu][^{nat}Zr] useful for therapy.

Compound D2-mAb[^{nat}Lu][⁸⁹Zr] and Compound D2-mAb[¹⁷⁷Lu][^{nat}Zr] will have identical pharmacokinetics and biodistribution properties, which is a useful property for scouting procedures. This approach would use natural Lu(III) and natural Zr(IV), which are both non-toxic metal ions.

1. A compound comprising:
a first chelating ligand selective for ⁸⁹Zr, and
a second chelating ligand selective for a nuclide of pharmaceutical potential other than ⁸⁹Zr,
wherein the first and second chelating ligands are covalently linked by a linker group.

2. The compound of claim 1, wherein the second chelating ligand is selective for ⁹⁰Y, ¹⁵³Sm, ¹⁶¹Tb, ¹⁷⁷Lu, ²¹³Bi and ²²⁵Ac or a combination thereof.

3. The compound of claim 1, wherein the chelating ligand selective for ⁸⁹Zr is also selective for ⁹⁰Nb.

4. The compound of claim 1, which is a compound of Formula (I)
A-L-B (I)
wherein
A is the first chelating ligand,
B is the second chelating ligand, and
L is a linker group.

5. The compound of claim 1, wherein the first chelating ligand comprises a hydroxamic acid group, or wherein the first chelating ligand is a hexadentate chelating ligand, or wherein the first chelating ligand is an octadentate chelating ligand.

6. (canceled)

7. (canceled)

8. The compound of claim 1, wherein the first chelating ligand is 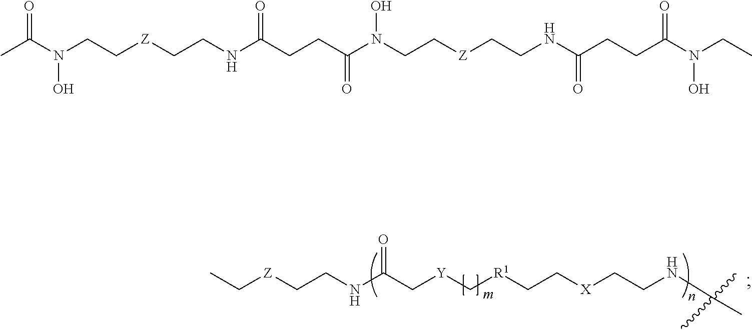
wherein R¹ is 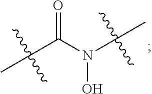 [SMILES: *C(=O)N(*)O]
Y is CH₂, O or S;
X is CH₂, O or S;
each Z is independently selected from CH₂ and O;
n is 0 or 1; and
m is 0 or 1.

9. The compound of claim 1, wherein the first chelating ligand is selected from the group consisting of:

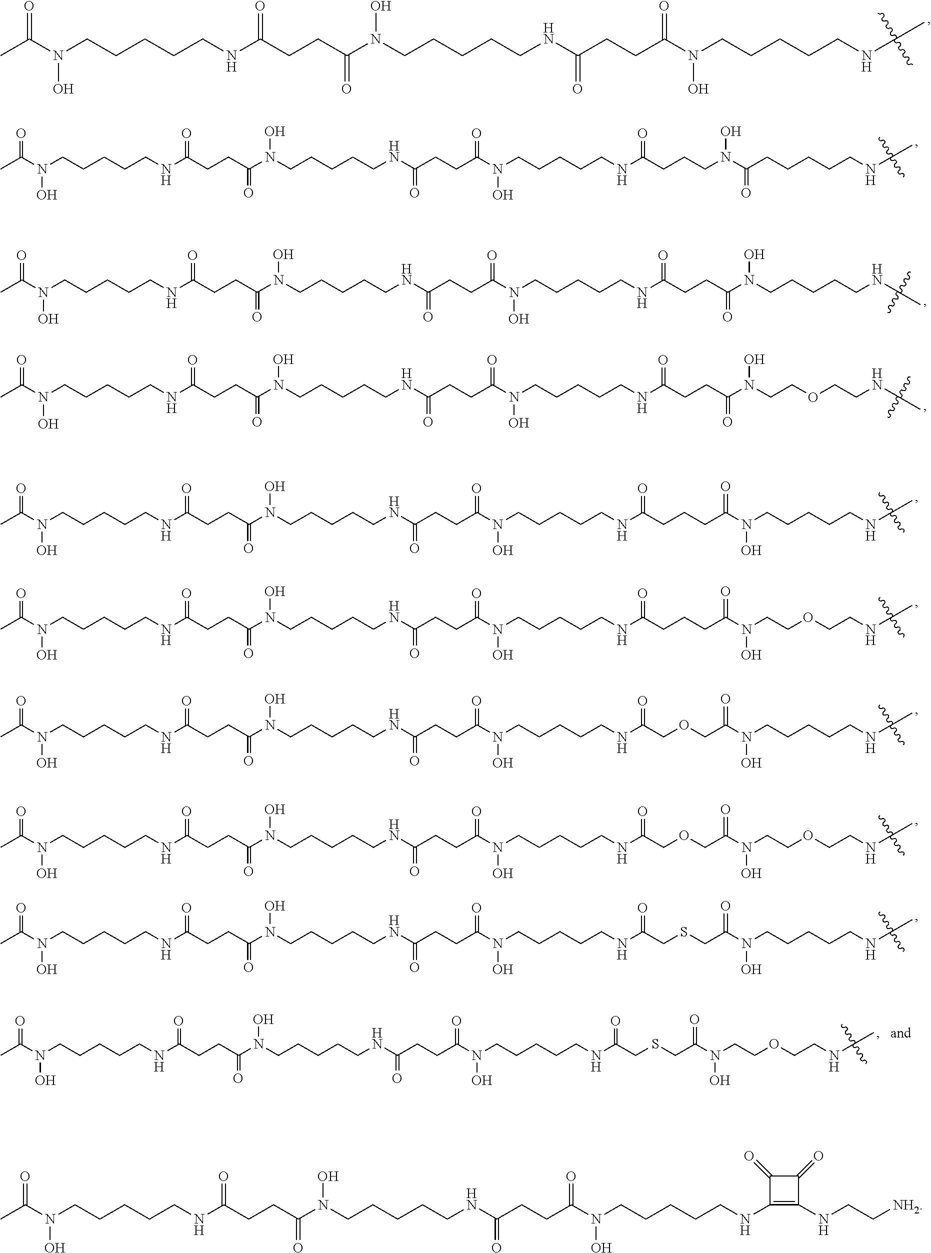

10. The compound of claim 1, wherein the second chelating ligand comprises a polyaminocarboxylic acid group, or wherein the second chelating ligand is selected from the grouvp consisting of

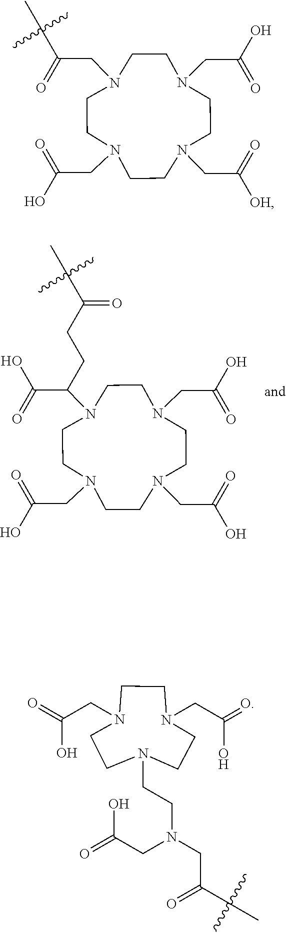 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1 ; *C(=O)CN(CCN1CCN(CC(=O)O)CCN(CC(=O)O)CC1)CC(=O)O]

11. (canceled)

12. A compound of formula (II)
Ch¹-L-Ch² (II)
wherein
Ch¹ is a radical of desferrioxamine B;
Ch² is a radical selected from the group consisting of 1,4,7,10-tetraazacyclododecane-1,4,7,10-tetraacetic acid (DOTA), alpha-(2-carboxyethyl)-1,4,7,10-tetraazacyclododecane-1,4,7,10-tetraacetic acid (DOTAGA) or 7-[2-[bis(carboxymethyl)amino]ethyl]hexahydro-1H-1,4,7-triazonine-1,4(5H)-diacetic acid (NETA); and
L is a linker group.

13. The compound of claim 1, wherein the linker group is a covalent bond, or wherein the linker group comprises a shortest linear chain of up to about 30 atoms.

14. (canceled)

15. The compound of claim 1, wherein the linker group is an optionally substituted C₁₋₂₀alkyl group optionally interrupted by one or more groups selected from a heteroatom, alkene, alkyne, cycloalkyl, heterocyclyl, amide, ester, ketone, targeting moiety or a group capable of forming a stable conjugate with a targeting group and a combination thereof.

16. The compound of claim 1, wherein the linker group comprises one or more amino acids, or wherein the linker group comprises L-lysine, L-glutamic acid, L-aspartic acid or combinations thereof.

17. (canceled)

18. The compound of claim 1, wherein the linker group is substituted with a targeting moiety or a substituent capable of conjugation to a targeting moiety.

19. The compound of claim 18, wherein the targeting moiety is girentuximab.

20. A complex comprising a compound of claim 1 and a nuclide of pharmaceutical potential or two different nuclides of pharmaceutical potential.

21. (canceled)

22. A composition comprising:
the compound of claim 1, and
a pharmaceutically acceptable excipient.

23. (canceled)

24. A method of treating a neoplastic disorder, comprising administering to a subject in need thereof a therapeutically effective amount of a complex of claim 20 to thereby treat the neoplastic disorder.

25. A method of diagnosing and/or prognosing a disease or condition, comprising administering to a subject in need thereof an effective amount of a complex of claim 20 and subjecting the subject to an imaging technique.

26. The method of claim 24, further comprising before the administering step, a step of contacting a compound comprising:
a first chelating ligand selective for ⁸⁹Zr, and a second chelating ligand selective for a nuclide of pharmaceutical potential other than ⁸⁹Zr, wherein the first and second chelating ligands are covalently linked by a linker group, with one or two nuclides of therapeutic or diagnostic potential.
